(19) United States
(12) Patent Application Publication
(10) Pub. No.: US 2018/0201921 A1
(43) Pub. Date: Jul. 19, 2018

(54) CRISPRs

(71) Applicant: EXCISION BIOTHERAPEUTICS, INC., Bedminster, NJ (US)

(72) Inventor: Thomas Malcolm, Bedminster, NJ (US)

(73) Assignee: EXCISION BIOTHERAPEUTICS, INC., Bedminster, NJ (US)

(21) Appl. No.: 15/872,471

(22) Filed: Jan. 16, 2018

Related U.S. Application Data
(60) Provisional application No. 62/447,472, filed on Jan. 18, 2017.

(51) Int. Cl. C12N 15/10; C12N 9/22

(52) U.S. Cl. CPC C12N 15/102; C12N 9/22; C12N 2310/20; C12N 2700/00; C12N 2780/00062; C12N 2710/00062; C12N 2720/00062; C12N 2760/00062; C12N 2770/00062; C12N 2310/141

(57) ABSTRACT

A composition for treating a lysogenic virus, including a vector encoding isolated nucleic acid encoding two or more gene editors chosen from gene editors that target viral DNA, gene editors that target viral RNA, and combinations thereof. A composition for treating a lytic virus, including a vector encoding isolated nucleic acid encoding at least one gene editor that targets viral DNA and a viral RNA targeting composition. A composition for treating both lysogenic and lytic viruses, including a vector encoding isolated nucleic acid encoding two or more gene editors that target viral RNA. A composition for treating lytic viruses. Methods of treating a lysogenic virus or a lytic virus, by administering the above compositions to an individual having a virus and inactivating the virus.

### BACKGROUND OF THE INVENTION

### 1. Technical Field

The present invention relates to compositions and methods for delivering gene therapeutics. More specifically, the present invention relates to compositions and treatments for excising viruses from infected host cells and inactivating viruses.

### 2. Background Art

Gene editing allows DNA or RNA to be inserted, deleted, or replaced in an organism's genome by the use of nucleases. There are several types of nucleases currently used, including meganucleases, zinc finger nucleases, transcription activator-like effector-based nucleases (TALENs), and clustered regularly interspaced short palindromic repeats (CRISPR)-Cas nucleases. These nucleases can create site-specific double strand breaks of the DNA in order to edit the DNA.

Meganucleases have very long recognition sequences and are very specific to DNA. While meganucleases are less toxic than other gene editors, they are expensive to construct, as not many are known and mutagenesis must be used to create variants that recognize specific sequences.

Both zinc-finger and TALEN nucleases are non-specific for DNA but can be linked to DNA sequence recognizing peptides. However, each of these nucleases can produce off-target effects and cytotoxicity, and require time to create the DNA sequence recognizing peptides.

CRISPR-Cas nucleases are derived from prokaryotic systems and can use either the Cas9 nuclease or the Cpf1 nuclease for DNA editing. CRISPR is an adaptive immune system found in many microbial organisms. While the CRISPR system was not well understood, it was found that there were genes associated to the CRISPR regions that coded for exonucleases and/or helicases, called CRISPR-associated proteins (Cas). Several different types of Cas proteins were found, some using multi-protein complexes (Type I), some using singe effector proteins with a universal tracrRNA and crRNA specific for a target DNA sequence (Type II), and some found in archea (Type III). Cas9 (a Type II Cas protein) was discovered when the bacteria Streptococcus thermophilus was being studied and an unusual CRISPR locus was found (Bolotin, et al. 2005). It was also found that the spacers share a common sequence at one end (the protospacer adjacent motif PAM), and is used for target sequence recognition. Cas9 was not found with a screen but by examining a specific bacteria.

U.S. patent application Ser. No. 14/838,057 to Khalili, et al. discloses a method of inactivating a proviral DNA integrated into the genome of a host cell latently infected with a retrovirus, by treating the host cell with a composition comprising a Clustered Regularly Interspaced Short Palindromic Repeat (CRISPR)-associated endonuclease, and two or more different guide RNAs (gRNAs), wherein each of the at least two gRNAs is complementary to a different target nucleic acid sequence in a long terminal repeat (LTR) of the proviral DNA; and inactivating the proviral DNA. A composition is also provided for inactivating proviral DNA. Delivery of the CRISPR-associated endonuclease and gRNAs can be by various expression vectors, such as plasmid vectors, lentiviral vectors, adenoviral vectors, or adeno-associated virus vectors.

Viruses replicate by one of two cycles, either the lytic cycle or the lysogenic cycle. In the lytic cycle, first the virus penetrates a host cell and releases its own nucleic acid. Next, the host cell's metabolic machinery is used to replicate the viral nucleic acid and accumulate the virus within the host cell. Once enough virions are produced within the host cell, the host cell bursts (lysis) and the virions go on to infect additional cells. Lytic viruses can integrate viral DNA into the host genome as well as be non-integrated where lysis does not occur over the period of the infection of the cell.

Lytic viruses include John Cunningham virus (JCV), hepatitis A, and various herpesviruses. In the lysogenic cycle, virion DNA is integrated into the host cell, and when the host cell reproduces, the virion DNA is copied into the resulting cells from cell division. In the lysogenic cycle, the host cell does not burst. Lysogenic viruses include hepatitis B, Zika virus, and HIV. Viruses such as lambda phage can switch between lytic and lysogenic cycles.

While the methods and compositions described above are useful in treating lysogenic viruses that have been integrated into the genome of a host cell, gene editing systems are not able to effectively treat lytic viruses. Treating a lytic virus will result in inefficient clearance of the virus if solely using this system unless inhibitor drugs are available to suppress viral expression, as in the case of HIV. Most viruses presently lack targeted inhibitor drugs. In particular, the CRISPR-associated nuclease cannot access viral nucleic acid that is contained within the virion (that is, protected by capsid or envelope proteins for example).

Researchers from the Broad Institute of MIT and Harvard, Mass. Institute of Technology, the National Institutes of Health, Rutgers University-New Brunswick and the Skolkovo Institute of Science and Technology have characterized a new CRISPR system that targets RNA, rather than DNA. This approach has the potential to open an additional avenue in cellular manipulation relating to editing RNA. Whereas DNA editing makes permanent changes to the genome of a cell, the CRISPR-based RNA-targeting approach can allow temporary changes that can be adjusted up or down, and with greater specificity and functionality than existing methods for RNA interference. Specifically, it can address RNA embedded viral infections and resulting disease. The study reports the identification and functional characterization of C2c2, an RNA-guided enzyme capable of targeting and degrading RNA.

The findings reveal that C2c2—the first naturally-occurring CRISPR system that targets only RNA to have been identified, discovered by this collaborative group in October 2015—helps protect bacteria against viral infection. They demonstrate that C2c2 can be programmed to cleave particular RNA sequences in bacterial cells, which would make it an important addition to the molecular biology toolbox. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner—and manipulate gene function more broadly. This has the potential to accelerate progress to understand, treat and prevent disease.

There remains a need for additional CRISPR enzymes for use in gene editing that can effectively target virus DNA or RNA.

### SUMMARY OF THE INVENTION

The present invention provides for a composition for treating a lysogenic virus including a vector encoding two or more gene editors chosen from the group consisting of gene editors that target viral DNA, gene editors that target viral RNA, and combinations thereof.

The present invention also provides for a composition for treating a lytic virus, including a vector encoding isolated nucleic acid encoding at least one gene editor that targets viral DNA and a viral RNA targeting composition.

The present invention also provides for a composition for treating both lysogenic and lytic viruses, including a vector encoding isolated nucleic acid encoding two or more gene editors that target viral RNA, chosen from the group consisting of CRISPR-associated nucleases, Argonaute endonuclease gDNAs, C2c2, RNase P RNA, and combinations thereof.

The present invention provides for a composition for treating lytic viruses, including a vector encoding isolated nucleic acid encoding two or more gene editors that target viral RNA and a viral RNA targeting composition.

The present invention provides for a method of treating a lysogenic virus, by administering a composition including a vector encoding isolated nucleic acid encoding two or more gene editors chosen from the group consisting of gene editors that target viral DNA, gene editors that target viral RNA, and combinations thereof to an individual having a lysogenic virus, and inactivating the lysogenic virus.

The present invention also provides for a method for treating a lytic virus, by administering a composition including a vector encoding isolated nucleic acid encoding at least one gene editor that targets viral DNA and a viral RNA targeting composition to an individual having a lytic virus, and inactivating the lytic virus.

The present invention also provides for a method for treating both lysogenic and lytic viruses, by administering a composition including a vector encoding isolated nucleic acid encoding two or more gene editors that target viral RNA, chosen from the group consisting of CRISPR-associated nucleases, Argonaute endonuclease gDNAs, C2c2, RNase P RNA, and combinations thereof to an individual having a lysogenic virus and lytic virus, and inactivating the lysogenic virus and lytic virus.

The present invention provides for a method for treating lytic viruses, by administering a composition including a vector encoding isolated nucleic acid encoding two or more gene editors that target viral RNA and a viral RNA targeting composition to an individual having a lytic virus, and inactivating the lytic virus.

### DESCRIPTION OF THE DRAWINGS

Other advantages of the present invention are readily appreciated as the same becomes better understood by reference to the following detailed description when considered in connection with the accompanying drawings wherein:

FIG. 1 is a picture of lytic and lysogenic virus within a cell and at which point CRISPR Cas9 can be used and at which point RNA targeting systems can be used; and

FIG. 2 is a chart of various Archaea Cas9 effectors, CasY.1-CasY.6 effectors, and CasX effectors of the present invention.

### DETAILED DESCRIPTION OF THE INVENTION

The present invention is generally directed to compositions and methods for treating lysogenic and lytic viruses with various gene editing systems and enzyme effectors. The compositions can treat both lysogenic viruses and lytic viruses, or optionally viruses that use both methods of replication.

The term “vector” includes cloning and expression vectors, as well as viral vectors and integrating vectors. An “expression vector” is a vector that includes a regulatory region. Vectors are also further described below.

The term “lentiviral vector” includes both integrating and non-integrating lentiviral vectors.

Viruses replicate by one of two cycles, either the lytic cycle or the lysogenic cycle. In the lytic cycle, first the virus penetrates a host cell and releases its own nucleic acid. Next, the host cell's metabolic machinery is used to replicate the viral nucleic acid and accumulate the virus within the host cell. Once enough virions are produced within the host cell, the host cell bursts (lysis) and the virions go on to infect additional cells. Lytic viruses can integrate viral DNA into the host genome as well as be non-integrated where lysis does not occur over the period of the infection of the cell. Viruses such as lambda phage can switch between lytic and lysogenic cycles.

“Lysogenic virus” as used herein, refers to a virus that replicates by the lysogenic cycle (i.e. does not cause the host cell to burst and integrates viral nucleic acid into the host cell DNA). The lysogenic virus can mainly replicate by the lysogenic cycle but sometimes replicate by the lytic cycle. In the lysogenic cycle, virion DNA is integrated into the host cell, and when the host cell reproduces, the virion DNA is copied into the resulting cells from cell division. In the lysogenic cycle, the host cell does not burst.

“Lytic virus” as used herein refers to a virus that replicates by the lytic cycle (i.e. causes the host cell to burst after an accumulation of virus within the cell). The lytic virus can mainly replicate by the lytic cycle but sometimes replicate by the lysogenic cycle.

“gRNA” as used herein refers to guide RNA. The gRNAs in the CRISPR Cas9 systems herein are used for the excision of viral genome segments and hence the crippling disruption of the virus' capability to replicate/produce protein. This is accomplished by using two or more specifically designed gRNAs to avoid the issues seen with single gRNAs such as viral escape or mutations. The gRNA can be a sequence complimentary to a coding or a non-coding sequence and can be tailored to the particular virus to be targeted. The gRNA can be a sequence complimentary to a protein coding sequence, for example, a sequence encoding one or more viral structural proteins, (e.g., gag, pol, env and tat). The gRNA sequence can be a sense or anti-sense sequence.

“Nucleic acid” as used herein, refers to both RNA and DNA, including cDNA, genomic DNA, synthetic DNA, and DNA (or RNA) containing nucleic acid analogs, any of which may encode a polypeptide of the invention and all of which are encompassed by the invention. Polynucleotides can have essentially any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, short hairpin RNA (shRNA), interfering RNA (RNAi), ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode a fragment of a naturally occurring Cas9 or a biologically active variant thereof and at least two gRNAs where in the gRNAs are complementary to a sequence in a virus.

An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a Cas9-encoding DNA (in accordance with, for example, the formula above).

There are many different gene editors (CRISPR systems or others) and enzyme effectors that can be used with the methods and compositions of the present invention to target either DNA or RNA in viruses. These include Argonaute proteins, RNase P RNA, C2c1, C2c2, C2c3, various Cas9 enzymes, Cpf1, TevCas9, Archaea Cas9, CasY.1-CasY.6 effectors, and CasX effectors. Each of these are further described below.

“Argonaute protein” as used herein, refers to proteins of the PIWI protein superfamily that contain a PIWI (P element-induced wimpy testis) domain, a MID (middle) domain, a PAZ (Piwi-Argonaute-Zwille) domain and an N-terminal domain. Argonaute proteins are capable of binding small RNAs, such as microRNAs, small interfering RNAs (siRNAs), and Piwi-interacting RNAs. Argonaute proteins can be guided to target sequences with these RNAs in order to cleave mRNA, inhibit translation, or induce mRNA degradation in the target sequence. There are several different human Argonaute proteins, including AGO1, AGO2, AGO3, and AGO4 that associate with small RNAs. AGO2 has slicer ability, i.e. acts as an endonuclease. Argonaute proteins can be used for gene editing. Endonucleases from the Argonaute protein family (from Natronobacterium gregoryi Argonaute) also use oligonucleotides as guides to degrade invasive genomes. Work by Gao et al has shown that the Natronobacterium gregoryi Argonaute (NgAgo) is a DNA-guided endonuclease suitable for genome editing in human cells. NgAgo binds 5′ phosphorylatedsingle-stranded guide DNA (gDNA) of ˜24 nucleotides, efficiently creates site-specific DNA double-strand breaks when loaded with the gDNA. The NgAgo-gDNA system does not require a protospacer-adjacent motif (PAM), as does Cas9, and preliminary characterization suggests a low tolerance to guide-target mismatches and high efficiency in editing (G+C)-rich genomic targets. The Argonaute protein endonucleases used in the present invention can also be Rhodobacter sphaeroides Argonaute (RsArgo). RsArgo can provide stable interaction with target DNA strands and guide RNA, as it is able to maintain base-pairing in the 3′-region of the guide RNA between the N-terminal and PIWI domains. RsArgo is also able to specifically recognize the 5′ base-U of guide RNA, and the duplex-recognition loop of the PAZ domain with guide RNA can be important in DNA silencing activity. Other prokaryotic Argonaute proteins (pAgos) can also be used in DNA interference and cleavage. The Argonaute proteins can be derived from Arabidopsis thaliana, D. melanogaster, Aquifex aeolicus, Thermus thermophiles, Pyrococcus furiosus, Thermus thermophilus JL-18, Thermus thermophilus strain HB27, Aquifex aeolicus strain VF5, Archaeoglobus fulgidus, Anoxybacillus flavithermus, Halogeometricum borinquense, Microsystis aeruginosa, Clostridium bartlettii, Halorubrum lacusprofundi, Thermosynechococcus elongatus, and Synechococcus elongatus. Argonaute proteins can also be used that are endo-nucleolytically inactive but post-translational modifications can be made to the conserved catalytic residues in order to activate them as endonucleases.

Human WRN is a RecQ helicase encoded by the Werner syndrome gene. It is implicated in genome maintenance, including replication, recombination, excision repair and DNA damage response. These genetic processes and expression of WRN are concomitantly upregulated in many types of cancers. Therefore, it has been proposed that targeted destruction of this helicase could be useful for elimination of cancer cells. Reports have applied the external guide sequence (EGS) approach in directing an RNase P RNA to efficiently cleave the WRN mRNA in cultured human cell lines, thus abolishing translation and activity of this distinctive 3′-5′ DNA helicase-nuclease. RNase P RNA is another potential endonuclease for use with the present invention.

The Class 2 type VI-A CRISPR/Cas effector “C2c2” demonstrates an RNA-guided RNase function. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner—and manipulate gene function more broadly. These results demonstrate the capability of C2c2 as a new RNA-targeting tools.

Another Class 2 type V-B CRISPR/Cas effector “C2c1” can also be used in the present invention for editing DNA. C2c1 contains RuvC-like endonuclease domains related distantly to Cpf1 (described below). C2c1 can target and cleave both strands of target DNA site-specifically. According to Yang, et al. (PAM-Dependent Target DNA Recognition and Cleavage by C2c1 CRISPR-Cas Endonuclease, Cell, 2016 Dec. 15; 167(7):1814-1828)), a crystal structure confirms Alicyclobacillus acidoterrestris C2c1 (AacC2c1) binds to sgRNA as a binary complex and targets DNAs as ternary complexes, thereby capturing catalytically competent conformations of AacC2c1 with both target and non-target DNA strands independently positioned within a single RuvC catalytic pocket. Yang, et al. confirms that C2c1-mediated cleavage results in a staggered seven-nucleotide break of target DNA, crRNA adopts a pre-ordered five-nucleotide A-form seed sequence in the binary complex, with release of an inserted tryptophan, facilitating zippering up of 20-bp guide RNA:target DNA heteroduplex on ternary complex formation, and that the PAM-interacting cleft adopts a “locked” conformation on ternary complex formation.

C2c3 is a gene editor effector of type V-C that is distantly related to C2c1, and also contains RuvC-like nuclease domains. C2c3 is also similar to the CasY.1-CasY.6 group described below.

“CRISPR Cas9” as used herein refers to Clustered Regularly Interspaced Short Palindromic Repeat (CRISPR)-associated endonuclease Cas9. In bacteria the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

CRISPR/Cpf1 is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. CRISPR/Cpf1 could have multiple applications, including treatment of genetic illnesses and degenerative conditions. As referenced above, Agonaute is another potential gene editing system.

A CRISPR/TevCas9 system can also be used. In some cases it has been shown that once CRISPR/Cas9 cuts DNA in one spot, DNA repair systems in the cells of an organism will repair the site of the cut. The TevCas9 enzyme was developed to cut DNA at two sites of the target so that it is harder for the cells' DNA repair systems to repair the cuts (Wolfs, et al., Biasing genome-editing events toward precise length deletions with an RNA-guided TevCas9 dual nuclease, PNAS, doi:10.1073). The TevCas9 nuclease is a fusion of a I-Tevi nuclease domain to Cas9.

The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Psuedomona aeruginosa, Escherichia coli, or other sequenced bacteria genomes and archaea, or other prokaryotic microorganisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine (2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins). The Cas-9 can also be any shown in TABLE 1 below.

TABLE 1

| Variant | | |
|---|---|---|
| No. | | Tested* |
| | Four Alanine Substitution Mutants | |
| | (compared to WT Cas9) | |
| 1 | SpCas9 N497A, R661A, Q695A, Q926A | YES |
| 2 | SpCas9 N497A, R661A, Q695A, Q926A + D1135E | YES |
| 3 | SpCas9 N497A, R661A, Q695A, Q926A + L169A | YES |
| 4 | SpCas9 N497A, R661A, Q695A, Q926A + Y450A | YES |
| 5 | SpCas9 N497A, R661A, Q695A, Q926A + M495A | Predicted |
| 6 | SpCas9 N497A, R661A, Q695A, Q926A + M694A | Predicted |
| 7 | SpCas9 N497A, R661A, Q695A, Q926A + H698A | Predicted |
| 8 | SpCas9 N497A, R661A, Q695A, Q926A + | Predicted |
| | D1135E + L169A | |
| 9 | SpCas9 N497A, R661A, Q695A, Q926A + | Predicted |
| | D1135E + Y450A | |
| 10 | SpCas9 N497A, R661A, Q695A, Q926A + | Predicted |
| | D1135E + M495A | |
| 11 | SpCas9 N497A, R661A, Q695A, Q926A + | Predicted |
| | D1135E + M694A | |
| 12 | SpCas9 N497A, R661A, Q695A, Q926A + | Predicted |
| | D1135E + M698A | |
| | Three Alanine Substitution Mutants | |
| | (compared to WT Cas9) | |
| 13 | SpCas9 R661A, Q695A, Q926A | No (on |
| | | target |
| | | only) |
| 14 | SpCas9 R661A, Q695A, Q926A + D1135E | Predicted |
| 15 | SpCas9 R661A, Q695A, Q926A + L169A | Predicted |
| 16 | SpCas9 R661A, Q695A, Q926A + Y450A | Predicted |
| 17 | SpCas9 R661A, Q695A, Q926A + M495A | Predicted |
| 18 | SpCas9 R661A, Q695A, Q926A + M694A | Predicted |
| 19 | SpCas9 R661A, Q695A, Q926A + H698A | Predicted |
| 20 | SpCas9 R661A, Q695A, Q926A + D1135E + | Predicted |
| | L169A | |
| 21 | SpCas9 R661A, Q695A, Q926A + D1135E + | Predicted |
| | Y450A | |
| 22 | SpCas9 R661A, Q695A, Q926A + D1135E + | Predicted |
| | M495A | |
| 23 | SpCas9 R661A, Q695A, Q926A + D1135E + | Predicted |
| | M694A | |

Although the RNA-guided endonuclease Cas9 has emerged as a versatile genome-editing platform, some have reported that the size of the commonly used Cas9 from Streptococcus pyogenes (SpCas9) limits its utility for basic research and therapeutic applications that use the highly versatile adeno-associated virus (AAV) delivery vehicle. Accordingly, the six smaller Cas9 orthologues have been used and reports have shown that Cas9 from Staphylococcus aureus (SaCas9) can edit the genome with efficiencies similar to those of SpCas9, while being more than 1 kilobase shorter. SaCas9 is 1053 bp, whereas SpCas9 is 1358 bp.

The Cas9 nuclease sequence, or any of the gene editor effector sequences described herein, can be a mutated sequence. For example the Cas9 nuclease can be mutated in the conserved HNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. In general, mutations of the gene editor effector sequence can minimize or prevent off-targeting.

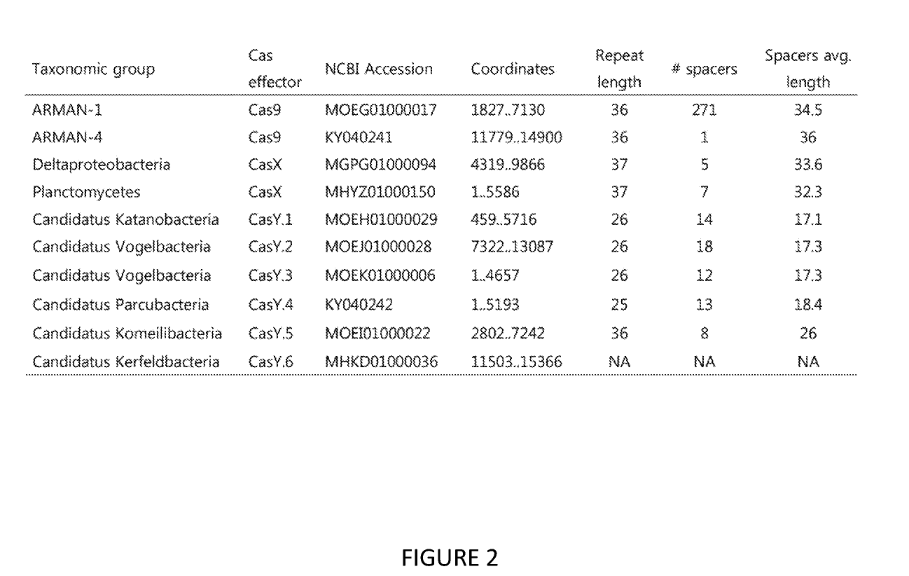

The gene editor effector can also be Archaea Cas9. The size of Archaea Cas9 is 950aa ARMAN 1 and 967aa ARMAN 4. The Archaea Cas9 can be derived from ARMAN-1 (Candidatus Micrarchaeum acidiphilum ARMAN-1) or ARMAN-4 (Candidatus Parvarchaeum acidiphilum ARMAN-4). Two examples of Archaea Cas9 are provided in FIG. 2, derived from ARMAN-1 and ARMAN-4. The sequences for ARMAN 1 and ARMAN 4 are below.

ARMAN 1 amino acid sequence 950 aa
(SEQ ID NO: 1):
MRDSITAPRYSSALAARIKEFNSAFKLGIDLGTKTGGVALVKDNKVLLAKTFLDYHKQTLEERRIHRRNRRSRL
ARRKRIARLRSWILRQKIYGKQLPDPYKIKKMQLPNGVRKGENWIDLVVSGRDLSPEAFVRAITLIFQKRGQRYEEVAKEI
EEMSYKEFSTHIKALTSVTEEEFTALAAEIERRQDVVDTDKEAERYTQLSELLSKVSESKSESKDRAQRKEDLGKVVNAFCS
AHRIEDKDKWCKELMKLLDRPVRHARFLNKVLIRCNICDRATPKKSRPDVRELLYFDTVRNFLKAGRVEQNPDVISYYKKI
YMDAEVIRVKILNKEKLTDEDKKQKRKLASELNRYKNKEYVTDAQKKMQEQLKTLLFMKLTGRSRYCMAHLKERAAGK
DVEEGLHGVVQKRHDRNIAQRNHDLRVINLIESLLFDQNKSLSDAIRKNGLMYVTIEAPEPKTKHAKKGAAVVRDPRKL
KEKLFDDQNGVCIYTGLQLDKLEISKYEKDHIFPDSRDGPSIRDNLVLTTKEINSDKGDRTPWEWMHDNPEKWKAFERR
VAEFYKKGRINERKRELLLNKGTEYPGDNPTELARGGARVNNFITEFNDRLKTHGVQELQTIFERNKPIVQVVRGEETQR
LRRQWNALNQNFIPLKDRAMSFNHAEDAAIAASMPPKFWREQIYRTAWHFGPSGNERPDFALAELAPQWNDFFMT
KGGPIIAVLGKTKYSWKHSIIDDTIYKPFSKSAYYVGIYKKPNAITSNAIKVLRPKLLNGEHTMSKNAKYYHQKIGNERFLM
KSQKGGSIITVKPHDGPEKVLQISPTYECAVLTKHDGKIIVKFKPIKPLRDMYARGVIKAMDKELETSLSSMSKHAKYKELH
THDIIYLPATKKHVDGYFIITKLSAKHGIKALPESMVKVKYTQIGSENNSEVKLTKPKPEITLDSEDITNIYNFTR
ARMAN 1 nucleic acid sequence
(SEQ ID NO: 2):
atga gagactctat tactgcacct agatacagct ccgctcttgc cgccagaata aaggagttta attctgcttt
caagttagga atcgacctag gaacaaaaac cggcggcgta gcactggtaa aagacaacaa agtgctgctc gctaagacat tcctcgatta
ccataaacaa acactggagg aaaggaggat ccatagaaga aacagaagga gcaggctagc caggcggaag aggattgctc ggctgcgatc
atggatactc agacagaaga tttatggcaa gcagcttcct gacccataca aaatcaaaaa aatgcagttg cctaatggtg tacgaaaagg
ggaaaactgg attgacctgg tagtttctgg acgggacctt tcaccagaag ccttcgtgcg tgcaataact ctgatattcc aaaagagagg
gcaaagatat gaagaagtgg ccaaagagat agaagaaatg agttacaagg aatttagtac tcacataaaa gccctgacat ccgttactga
agaagaattt actgctctgg cagcagagat agaacggagg caggatgtgg ttgacacaga caaggaggcc gaacgctata cccaattgtc
tgagttgctc tccaaggtct cagaaagcaa atctgaatct aaagacagag cgcagcgtaa ggaggatctc ggaaaggtgg tgaacgcttt
ctgcagtgct catcgtatcg aagacaagga taaatggtgt aaagaactta tgaaattact agacagacca gtcagacacg ctaggttcct
taacaaagta ctgatacgtt gcaatatctg cgatagggca acccctaaga aatccagacc tgacgtgagg gaactgctat attttgacac
agtaagaaac ttcttgaagg ctggaagagt ggagcaaaac ccagacgtta ttagttacta taaaaaaatt tatatggatg cagaagtaat
cagggtcaaa attctgaata aggaaaagct gactgatgag gacaaaaagc aaaagaggaa attagcgagc gaacttaaca ggtacaaaaa
caaagaatac gtgactgatg cgcagaagaa gatgcaagag caacttaaga cattgctgtt catgaagctg acaggcaggt ctagatactg
catggctcat cttaaggaaa gggcagcagg caaagatgta gaagaaggac ttcatggcgt tgtgcagaaa agacacgaca ggaacatagc
acagcgcaat cacgacttac gtgtgattaa tcttattgag agtctgcttt tcgaccaaaa caaatcgctc tccgatgcaa taaggaagaa
cgggttaatg tatgttacta ttgaggctcc agagccaaag actaagcacg caaagaaagg cgcagctgtg gtaagggatc ccagaaagtt
gaaggagaag ttgtttgatg atcaaaacgg cgtttgcata tatacgggct tgcagttaga caaattagag ataagtaaat acgagaagga
ccatatcttt ccagattcaa gggatggacc atctatcagg gacaatcttg tactcactac aaaagagata aattcagaca aaggcgatag
gaccccatgg gaatggatgc atgataaccc agaaaaatgg aaagcgttcg agagaagagt cgcagaattc tataagaaag gcagaataaa
tgagaggaaa agagaactcc tattaaacaa aggcactgaa taccctggcg ataacccgac tgagctggcg cggggaggcg cccgtgttaa
caactttatt actgaattta atgaccgcct caaaacgcat ggagtccagg aactgcagac catctttgag cgtaacaaac caatagtgca
ggtagtcagg ggtgaagaaa cgcagcgtct gcgcagacaa tggaatgcac taaaccagaa tttcatacca ctaaaggaca gggcaatgtc
gttcaaccac gctgaagacg cagccatagc agcaagcatg ccaccaaaat tctggaggga gcagatatac cgtactgcgt ggcactttgg
acctagtgga aatgagagac cggactttgc tttggcagaa ttggcgccac aatggaatga cttctttatg actaagggcg gtccaataat
agcagtgctg ggcaaaacga agtatagttg gaagcacagc ataattgatg acactatata caagccattc agcaaaagtg cttactatgt
tgggatatac aaaaagccga acgccatcac gtccaatgct ataaaagtct taaggccaaa actcttaaat ggcgaacata caatgtctaa
gaatgcaaag tattatcatc agaagattgg taatgagcgc ttcctcatga aatctcagaa aggtggatcg ataattacag taaaaccaca
cgacggaccg gaaaaagtgc ttcaaatcag ccctacatat gaatgcgcag tccttactaa gcatgacggt aaaataatag tcaaatttaa
accaataaag ccgctacggg acatgtatgc ccgcggtgtg attaaagcca tggacaaaga gcttgaaaca agcctctcta gcatgagtaa
acacgctaag tacaaggagt tacacactca tgatatcata tatctgcctg ctacaaagaa gcacgtagat ggctacttca taataaccaa
actaagtgcg aaacatggca taaaagcact ccccgaaagc atggttaaag tcaagtatac tcaaattggg agtgaaaaca atagtgaagt
gaagcttacc aaaccaaaac cagagataac tttggatagt gaagatatta caaacatata taatttcacc cgctaag
ARMAN 4 amino acid sequence 967 aa
(SEQ ID NO: 3):
MLGSSRYLRYNLTSFEGKEPFLIMGYYKEYNKELSSKAQKEFNDQISEFNSYYKLGIDLGDKTGIAIVKGNKIIL
AKTLIDLHSQKLDKRREARRNRRTRLSRKKRLARLRSWVMRQKVGNQRLPDPYKIMHDNKYWSIYNKSNSANKKNWI
DLLIHSNSLSADDFVRGLTIIFRKRGYLAFKYLSRLSDKEFEKYIDNLKPPISKYEYDEDLEELSSRVENGEIEEKKFEGLKNKL
DKIDKESKDFQVKQREEVKKELEDLVDLFAKSVDNKIDKARWKRELNNLLDKKVRKIRFDNRFILKCKIKGCNKNTPKKEK
VRDFELKMVLNNARSDYQISDEDLNSFRNEVINIFQKKENLKKGELKGVTIEDLRKQLNKTFNKAKIKKGIREQIRSIVFEKI
SGRSKFCKEHLKEFSEKPAPSDRINYGVNSAREQHDFRVLNFIDKKIFKDKLIDPSKLRYITIESPEPETEKLEKGQISEKSFET
LKEKLAKETGGIDIYTGEKLKKDFEIEHIFPRARMGPSIRENEVASNLETNKEKADRTPWEWFGQDEKRWSEFEKRVNSL
YSKKKISERKREILLNKSNEYPGLNPTELSRIPSTLSDFVESIRKMFVKYGYEEPQTLVQKGKPIIQVVRGRDTQALRWRW
HALDSNIIPEKDRKSSFNHAEDAVIAACMPPYYLRQKIFREEAKIKRKVSNKEKEVTRPDMPTKKIAPNWSEFMKTRNEP
VIEVIGKVKPSWKNSIMDQTFYKYLLKPFKDNLIKIPNVKNTYKWIGVNGQTDSLSLPSKVLSISNKKVDSSTVLLVHDKK
GGKRNWVPKSIGGLLVYITPKDGPKRIVQVKPATQGLLIYRNEDGRVDAVREFINPVIEMYNNGKLAFVEKENEEELLKY
FNLLEKGQKFERIRRYDMITYNSKFYYVTKINKNHRVTIQEESKIKAESDKVKSSSGKEYTRKETEELSLQKLAELISI
ARMAN 4 nucleic acid sequence
(SEQ ID NO: 4):
at gttaggctcc agcaggtacc tccgttataa cctaacctcg tttgaaggca aggagccatt tttaataatg ggatattaca
aagagtataa taaggaatta agttccaaag ctcaaaaaga atttaatgat caaatttctg aatttaattc gtattacaaa ctaggtatag
atctcggaga taaaacagga attgcaatcg taaagggcaa caaaataatc ctagcaaaaa cactaattga tttgcattcc caaaaattag
ataaaagaag ggaagctaga agaaatagaa gaactcggct ttccagaaag aaaaggcttg cgagattaag atcgtgggta atgcgtcaga
aagttggcaa tcaaagactt cccgatccat ataaaataat gcatgacaat aagtactggt ctatatataa taagagtaat tctgcaaata
aaaagaattg gatagatctg ttaatccaca gtaactcttt atcagcagac gattttgtta gaggcttaac tataattttc agaaaaagag
gctatttagc atttaagtat ctttcaaggt taagcgataa ggaatttgaa aaatacatag ataacttaaa accacctata agcaaatacg
agtatgatga ggatttagaa gaattatcaa gcagggttga aaatggggaa atagaggaaa agaaattcga aggcttaaag aataagctag
ataaaataga caaagaatct aaagactttc aagtaaagca aagagaagaa gtaaaaaagg aactggaaga cttagttgat ttgtttgcta
aatcagttga taataaaata gataaagcta ggtggaaaag ggagctaaat aatttattgg ataagaaagt aaggaaaata cggtttgaca
accgctttat tttgaagtgc aaaattaagg gctgtaacaa gaatactcca aagaaagaga aggtcagaga ttttgaattg aagatggttt
taaataatgc tagaagcgat tatcagattt ctgatgagga tttaaactct tttagaaatg aagtaataaa tatatttcaa aagaaggaaa
acttaaagaa aggagagctg aaaggagtta ctattgaaga tttgagaaag cagcttaata aaacttttaa taaagccaag attaaaaaag
ggataaggga gcagataagg tctatcgtgt ttgaaaaaat tagtggaagg agtaaattct gcaaagaaca tctaaaagaa ttttctgaga
agccggctcc ttctgacagg attaattatg gggttaattc agcaagagaa caacatgatt ttagagtctt aaatttcata gataaaaaaa
tattcaaaga taagttgata gatccctcaa aattgaggta tataactatt gaatctccag aaccagaaac agagaagttg gaaaaaggtc
aaatatcaga gaagagcttc gaaacattga aagaaaaatt ggctaaagaa acaggtggta ttgatatata cactggtgaa aaattaaaga
aagactttga aatagagcac atattcccaa gagcaaggat ggggccttct ataagggaaa acgaagtagc atcaaatctg gaaacaaata
aggaaaaggc cgatagaact ccttgggaat ggtttgggca agatgaaaaa agatggtcag agtttgagaa aagagttaat tctctttata
gtaaaaagaa aatatcagag agaaaaagag aaattttgtt aaataagagt aatgaatatc cgggattaaa ccctacagaa ctaagtagaa
tacctagtac gctgagcgac ttcgttgaga gtataagaaa aatgtttgtt aagtatggct atgaagagcc tcaaactttg gttcaaaaag
gaaaaccgat aatacaagtt gttagaggca gagacacaca agctttgagg tggagatggc atgcattaga tagtaatata ataccagaaa
aggacaggaa aagttcattt aatcacgctg aagatgcagt tattgccgcc tgtatgccac cttactatct caggcaaaaa atatttagag
aagaagcaaa aataaaaaga aaagtaagca ataaggaaaa ggaagttaca cggcctgaca tgcctactaa aaagatagct ccgaactggt
cggaatttat gaaaactaga aatgagccgg ttattgaagt aataggaaaa gttaagccaa gctggaaaaa cagcataatg gatcaaacat
tttataaata tcttttgaag ccatttaaag ataacctgat aaaaataccc aacgttaaaa atacatacaa gtggatagga gttaatggac
aaactgattc attatccctc ccgagtaagg tcttatctat ctctaataaa aaggttgatt cttctacagt tcttcttgtg catgataaga
agggtggtaa gcggaattgg gtacctaaaa gtataggggg tttgttggta tatataactc ctaaagacgg gccgaaaaga atagttcaag
taaagccagc aactcagggt ttgttaatat atagaaatga agatggcaga gtagatgctg taagagagtt cataaatcca gtgatagaaa
tgtataataa tggcaaattg gcatttgtag aaaaagaaaa tgaagaagag cttttgaaat attttaattt gctggaaaaa ggtcaaaaat
ttgaaagaat aagacggtat gatatgataa cctacaatag taaattttac tatgtaacaa aaataaacaa gaatcacaga gttactatac
aagaagagtc taagataaaa gcagaatcag acaaagttaa gtcctcttca ggcaaagagt atactcgtaa ggaaaccgag gaattatcac
ttcaaaaatt agcggaatta attagtatat aaaa

The gene editor effector can also be CasX, examples of which are shown in FIG. 2. CasX has a TTC PAM at the 5′ end (similar to Cpf1). The TTC PAM can have limitations in viral genomes that are GC rich, but not so much in those that are GC poor. The size of CasX (986 bp), smaller than other type V proteins, provides the potential for four gRNA plus one siRNA in a delivery plasmid. CasX can be derived from Deltaproteobacteria or Planctomycetes. The sequences for these CasX effectors are below.

CasX.1 Planctomycetes amino acid sequence 978 aa
(SEQ ID NO: 5):
MQEIKRINKIRRRLVKDSNTKKAGKTGPMKTLLVRVMTPDLRERLENLRKKPENIPQPISNTSRANLNKLLTD
YTEMKKAILHVYWEEFQKDPVGLMSRVAQPAPKNIDQRKLIPVKDGNERLTSSGFACSQCCQPLYVYKLEQVNDKGKP
HTNYFGRCNVSEHERLILLSPHKPEANDELVTYSLGKFGQRALDFYSIHVTRESNHPVKPLEQIGGNSCASGPVGKALSD
ACMGAVASFLTKYQDIILEHQKVIKKNEKRLANLKDIASANGLAFPKITLPPQPHTKEGIEAYNNVVAQIVIWVNLNLWQ
KLKIGRDEAKPLQRLKGFPSFPLVERQANEVDWWDMVCNVKKLINEKKEDGKVFWQNLAGYKRQEALLPYLSSEEDRK
KGKKFARYQFGDLLLHLEKKHGEDWGKVYDEAWERIDKKVEGLSKHIKLEEERRSEDAQSKAALTDWLRAKASFVIEGL
KEADKDEFCRCELKLQKWYGDLRGKPFAIEAENSILDISGFSKQYNCAFIWQKDGVKKLNLYLIINYFKGGKLRFKKIKPEA
FEANRFYTVINKKSGEIVPMEVNFNFDDPNLIILPLAFGKRQGREFIWNDLLSLETGSLKLANGRVIEKTLYNRRTRQDEP
ALFVALTFERREVLDSSNIKPMNLIGIDRGENIPAVIALTDPEGCPLSRFKDSLGNPTHILRIGESYKEKQRTIQAAKEVEQR
RAGGYSRKYASKAKNLADDMVRNTARDLLYYAVTQDAMLIFENLSRGFGRQGKRTFMAERQYTRMEDWLTAKLAYE
GLPSKTYLSKTLAQYTSKTCSNCGFTITSADYDRVLEKLKKTATGWMTTINGKELKVEGQITYYNRYKRQNVVKDLSVELD
RLSEESVNNDISSWTKGRSGEALSLLKKRFSHRPVQEKFVCLNCGFETHADEQAALNIARSWLFLRSQEYKKYQTNKTTG
NTDKRAFVETWQSFYRKKLKEVWKPAV
CasX.1 Planctomycetes nucleic acid sequence
(SEQ ID NO: 6):
atgct tcttatttat cggagatatc ttcaaacacc atcaacatgg caatggtgaa ccattaatat tctttgatgc ttcttattta
tcggagatat cttcaaacat tgcccatttt acaggcatat cttctggctc tttgatgctt cttatttatc ggagatatct tcaaacgtaa
tgtattgaga aagacatcaa gattagataa ctttgatgct tcttatttat cggagatatc ttcaaacaca gaaacctgca aagattgtat
atatataagc tttgatgctt cttatttatc ggagatatct tcaaacgata cgtattttag cccgtctatt tggggattaa ctttgatgct
tcttatttat cggagatatc ttcaaacccc gcatatccag atttttcaat gacttctgga aattgtattt tcaatatttt acaagttgcg
gaggatacct ttaataattt agcagagtta cgcactgtaa acctgttctt ctcacaaaaa gctttaacat cagattttca aagaacttct
tatgtaattt ataagaatct aaaaaaacag ctctgggttt gcatccagaa ctctccgata aataagcgct ttacccatac gacatagtcg
ctggtgatgg ctctcaaagt aatgagataa aagcgccagt aataatttac tattcacaaa tcctttcgtc aagcttaaaa tcaatcaaag
accatatccc cttcattcca aatagcagcg cttccgtacc tttctatccg ttcatatatc tcctctgaga gaggataaat taccagactt
atagagccat ccataaatcc tttttcttta aggttgagct ttagatcagc ccaccttgct tttgaaaggt taaactcaaa gacagaatat
tgaatccgaa caccataggc ttccagaagt ttaactaacc gtgccctgac cttatcatct tcaatatcat aacaaatgag atgtcgcatt
ttaaagctct ataggcttat aacattccct atcatcttga atatgctggc taaacaacct aacctgccgc tcaactgcgt gctgatacgt
tattgattgg ataagtaaat tggttttctg ctcatctacc ttaaagaatt gatgccattt tttgattact tttggatagg catccttatt
cagccaaaca cctttttggt cagtttcttt cctgaaatcg tctgtatcca cttcccttct atttatcaaa ttgatcacaa aacggtcagc
caacggccgc cactcctcca gaagatcgca tattaaagag ggacgaccat aatagacgtc atgcaagtaa ccaaaggccg ggtcaaaacc
gacgagtaat gcagtcgaat gtatttcgtt gaacaggagg gtgtagataa ggctcatcat ggcgttgatt tcatcctcag gaggtctctt
ggtacggcgc acaaaaacaa agcttggatg ctttaagata gccgaaaaat tgccataata ctgccttgtt gttgcgcctt ctattccacg
caaggtctct aaatcagtga cggcgttgat ttcggtacac tcgattctca aaccaagtct atatttatca agtaatgatt gctggttttt
gatcttaccg gcaacgatac tttttgcaat ttcaagtttt ttgtggggat caaaatgctt atgaatttgc gcccgacgaa taaacagatt
tttgacgggt tcaaattgaa ggctcccttg atattcccat ctgccgctaa agaaatgtat cggtatagat tattctctgc aaaggctaat
aacacggcta tcgagggtaa cccggccaac taccacgata tcttttacct tcattgcggg aatcttctgc cccttctctt cattgtcctt
ttttatgaga aatgcccgac cacgacaatc caaaatgaat tcatcacccg tgagatagag ggttatcctg tcggttatag cggtcatcag
taagcctttt atttttctaa ccaagtattg aaggaagaca cgattcacta tactggcact gcggacacct atggtcatca accttgggaa
acctgcttat atcaaaggac aagaagcagt ctcgcagatt tgtaacaact tctacacaac gcactttcag ggttttatct ataacaattt
ctttccgtct ccgtgtttca cagaaaaata tttcaccaac tggtatattg acattataca tctcttcaag gcaaattgcc tgtaacccaa
tctgaacgtg gaagttctca aaatccctta ccttccctgt ctttgtttcg ataggaatcg gtatcccatc cctccactcg ataaggtctg
cccggcctgc caaaccgagc ttattgctgt aaagatacac gcctgttacc tgcttacaat cagggcagct tctctgcgat gatttatcca
ccgccctgtg cgcgtgtatg gcctctgtaa agtggatgct cttagccata ttacgccgtt ctccaacaaa ggcataccat gcattgcgcg
gacaatagat tgactccatt accgtgctga tgtgcaatat cagacggctg gtttccatac ttctttgagc ttctttctgt aaaaggattg
ccatgtttca acaaatgccc ttttgtcagt atttccggtc gttttattgg tttgatacttcttatattct tgagaacgga gaaagagcca
cgaccttgca atattcagtg ctgcttgttc gtctgcatgg gtttcaaaac cacagttcag gcaaacaaac ttttcctgca ccggcctgtg
actaaatctc ttttttagca gagataaagc ttcaccactg cggccttttg tccaactaga aatatcatta tttaccgact cttccgaaag
tctatccagc tctacagaga ggtcttttac cacattctgc cttttatacc ggttatagta tgttatctgt ccttcaactt ttaactcttt
tccattgatt gtagtcatcc atccagtagc cgtcttcttg agcttttcga gcaccctgtc ataatctgca cttgtgattg taaaaccaca
attagaacat gtctttgagg tatactgtgc cagagtcttt gaaagatagg tttttgatgg cagaccttca taggcaagct ttgcagtcag
ccagtcttcc atcctcgtgt actgcctttc cgccataaaa gtcctcttgc cttgtctacc aaaaccgcgg gaaagatttt caaaaatgag
cattgcatct tgagtaacag cataatataa gaggtcacga gctgtatttc ttaccatatc gtccgccaga ttcttcgcct ttgatgcata
ttttctcgaa tatccgcctg cccgcctttg ttcaacttct ttagcagcct gaatagtccg ttgtttttcc ttataacttt ctcctattcg
caaaatatgc gttggattgc ccaatgaatc tttgaatctt gacaaggggc atccttccgg gtctgttaat gctatgactg ccgggatatt
ttctccccgg tctattccta tcagattcat cggttttata ttcgatgagt caagcacctc tcttctttca aatgtcaggg caacaaaaag
tgctggttca tcctgtctcg tccttctgtt atagagcgtt ttttcaataa ccctgccatt ggcgagtttc aatgaacccg tctcaaggct
caataggtcg ttccagataa actccctccc ctgccttttt ccaaaggcca aaggcagaat tatcaaattc gggtcatcaa aattgaagtt
gacctccata ggcacaatct caccgctttt tttattaatt actgtataaa acctatttgc ttcaaaagct tctggcttga tttttttgaa
gcgtagctta ccacctttga agtaatttat tattaaataa agatttaact tctttacgcc gtctttctgc catataaatg cacaattata
ctgtttagaa aatccgctta tatctaaaat gctgttctct gcttctatag caaatggttt tcctctcaaa tctccatacc acttttgaag
ctttaactca cacctgcaaa actcatcctt atcagcttct ttgagccctt caataacaaa agaggccttt gccctgagcc aatcagtgag
ggcagccttt gattgagcat cttcagacct tctttcttcc tccaacttta tgtgcttact cagaccttca acttttttat ctattctttc
ccatgcctca tcataaactt tgccccaatc ttcaccgtgt ttcttttcaa ggtgaagcaa aaggtcacca aactgataac gcgcaaactt
ttttcctttt ttacggtctt cttcagacga aagatatgga agcaaggctt cctgcctttt atatccagca agattttgcc agaagacctt
cccgtcctct ttcttttcgt taatcaactt tttgacatta cagaccatat cccaccaatc aacctcattc gcctggcgtt caacaagagg
gaaggacgga aaacccttaa gccgctgtaa gggctttgcc tcatccctgc caattttgag tttctgccaa agattcaggt ttacccagat
cactatctga gcaacaacat tgttataagc ttcaatccct tcttttgtat gcggttgcgg tggaagagtg attttaggaa atgcaagccc
gtttgcactt gctatatcct ttagatttgc caatctcttt tcgttttttt ttataacctt ttggtgttcg aggatgatgt cctggtactt
tgtaaggaaa ctggctactg ctcccataca ggcatcagat aaagccttac caacgggacc acttgcgcag ctattgccac cgatctgttc
tagcggcttt acaggatggt tcgattctct tgttacgtgg attgaataaa agtccaatgc cctttgaccg aacttcccca acgaatacgt
tactagctcg tcatttgcct ccggtttatg cggcgagagc aatatcaaac gttcatgctc ggagacatta caacggccaa agtaatttgt
atggggctta cccttgtcat tcacttgttc aagcttataa acatagaggg gttgacagca ctgagaacag gcaaatccag aacttgttag
tctctcattt ccgtccttca ccggaatcaa ttttctctga tcaatattct tgggcgctgg ttgtgcaacc ctgctcatca atccgacagg
gtctttttgg aactcttccc aataaacatg caggattgct ttcttcattt ccgtatagtc agtgaggagt ttatttaaat ttgcacgtga
agtatttgaa atgggctgag gaatgttttc cggctttttg cgaagattct ctaacctttc tctcaggtca ggtgtcataa cccgaacgag
caaggttttc atagggccgg ttttgccggc ttttttcgtg ttgctatcct ttaccaatct ccttcgtatt ttatttatcc tttttatttc
ctgcatcttt
CasX.1 Deltaproteobacteria amino acid sequence 986 aa
(SEQ ID NO: 7):
MEKRINKIRKKLSADNATKPVSRSGPMKTLLVRVMTDDLKKRLEKRRKKPEVMPQVISNNAANNLRMLLD
DYTKMKEAILQVYWQEFKDDHVGLMCKFAQPASKKIDQNKLKPEMDEKGNLTTAGFACSQCGQPLFVYKLEQVSEKG
KAYTNYFGRCNVAEHEKLILLAQLKPEKDSDEAVTYSLGKFGQRALDFYSIHVTKESTHPVKPLAQIAGNRYASGPVGKAL
SDACMGTIASFLSKYQDIIIEHQKVVKGNQKRLESLRELAGKENLEYPSVTLPPQPHTKEGVDAYNEVIARVRMWVNLN
LWQKLKLSRDDAKPLLRLKGFPSFPVVERRENEVDWWNTINEVKKLIDAKRDMGRVFWSGVTAEKRNTILEGYNYLPN
ENDHKKREGSLENPKKPAKRQFGDLLLYLEKKYAGDWGKVFDEAWERIDKKIAGLTSHIEREEARNAEDAQSKAVLTD
WLRAKASFVLERLKEMDEKEFYACEIQLQKWYGDLRGNPFAVEAENRVVDISGFSIGSDGHSIQYRNLLAWKYLENGKR
EFYLLMNYGKKGRIRFTDGTDIKKSGKWQGLLYGGGKAKVIDLTFDPDDEQLIILPLAFGTRQGREFIWNDLLSLETGLIK
LANGRVIEKTIYNKKIGRDEPALFVALTFERREVVDPSNIKPVNLIGVDRGENIPAVIALTDPEGCPLPEFKDSSGGPTDILR
IGEGYKEKQRAIQAAKEVEQRRAGGYSRKFASKSRNLADDMVRNSARDLFYHAVTHDAVLVFENLSRGFGRQGKRTF
MTERQYTKMEDWLTAKLAYEGLTSKTYLSKTLAQYTSKTCSNCGFTITTADYDGMLVRLKKTSDGWATTLNNKELKAE
GQITYYNRYKRQTVEKELSAELDRLSEESGNNDISKWTKGRRDEALFLLKKRFSHRPVQEQFVCLDCGHEVHADEQAAL
NIARSWLFLNSNSTEFKSYKSGKQPFVGAWQAFYKRRLKEVWKPNA
CasX.1 Deltaproteobacteria nucleic acid sequence
(SEQ ID NO: 8):
at ggaaaagaga ataaacaaga tacgaaagaa actatcggcc gataatgcca caaagcctgt gagcaggagc
ggccccatga aaacactcct tgtccgggtc atgacggacg acttgaaaaa aagactggag aagcgtcgga aaaagccgga agttatgccg
caggttattt caaataacgc agcaaacaat cttagaatgc tccttgatga ctatacaaag atgaaggagg cgatactaca agtttactgg
caggaattta aggacgacca tgtgggcttg atgtgcaaat ttgcccagcc tgcttccaaa aaaattgacc agaacaaact aaaaccggaa
atggatgaaa aaggaaatct aacaactgcc ggttttgcat gttctcaatg cggtcagccg ctatttgttt ataagcttga acaggtgagt
gaaaaaggca aggcttatac aaattacttc ggccggtgta atgtggccga gcatgagaaa ttgattcttc ttgctcaatt aaaacctgaa
aaagacagtg acgaagcagt gacatactcc cttggcaaat tcggccagag ggcattggac ttttattcaa tccacgtaac aaaagaatcc
acccatccag taaagcccct ggcacagatt gcgggcaacc gctatgcaag cggacctgtt ggcaaggccc tttccgatgc ctgtatgggc
actatagcca gttttctttc gaaatatcaa gacatcatca tagaacatca aaaggttgtg aagggtaatc aaaagaggtt agagagtctc
agggaattgg cagggaaaga aaatcttgag tacccatcgg ttacactgcc gccgcagccg catacgaaag aaggggttga cgcttataac
gaagttattg caagggtacg tatgtgggtt aatcttaatc tgtggcaaaa gctgaagctc agccgtgatg acgcaaaacc gctactgcgg
ctaaaaggat tcccatcttt ccctgttgtg gagcggcgtg aaaacgaagt tgactggtgg aatacgatta atgaagtaaa aaaactgatt
gacgctaaac gagatatggg acgggtattc tggagcggcg ttaccgcaga aaagagaaat accatccttg aaggatacaa ctatctgcca
aatgagaatg accataaaaa gagagagggc agtttggaaa accctaagaa gcctgccaaa cgccagtttg gagacctctt gctgtatctt
gaaaagaaat atgccggaga ctggggaaag gtcttcgatg aggcatggga gaggatagat aagaaaatag ccggactcac aagccatata
gagcgcgaag aagcaagaaa cgcggaagac gctcaatcca aagccgtact tacagactgg ctaagggcaa aggcatcatt tgttcttgaa
agactgaagg aaatggatga aaaggaattc tatgcgtgtg aaatccaact tcaaaaatgg tatggcgatc ttcgaggcaa cccgtttgcc
gttgaagctg agaatagagt tgttgatata agcgggtttt ctatcggaag cgatggccat tcaatccaat acagaaatct ccttgcctgg
aaatatctgg agaacggcaa gcgtgaattc tatctgttaa tgaattatgg caagaaaggg cgcatcagat ttacagatgg aacagatatt
aaaaagagcg gcaaatggca gggactatta tatggcggtg gcaaggcaaa ggttattgat ctgactttcg accccgatga tgaacagttg
ataatcctgc cgctggcctt tggcacaagg caaggccgcg agtttatctg gaacgatttg ctgagtcttg aaacaggcct gataaagctc
gcaaacggaa gagttatcga aaaaacaatc tataacaaaa aaatagggcg ggatgaaccg gctctattcg ttgccttaac atttgagcgc
cgggaagttg ttgatccatc aaatataaag cctgtaaacc ttataggcgt tgaccgcggc gaaaacatcc cggcggttat tgcattgaca
gaccctgaag gttgtccttt accggaattc aaggattcat cagggggccc aacagacatc ctgcgaatag gagaaggata taaggaaaag
cagagggcta ttcaggcagc aaaggaggta gagcaaaggc gggctggcgg ttattcacgg aagtttgcat ccaagtcgag gaacctggcg
gacgacatgg tgagaaattc agcgcgagac cttttttacc atgccgttac ccacgatgcc gtccttgtct ttgaaaacct gagcaggggt
tttggaaggc agggcaaaag gaccttcatg acggaaagac aatatacaaa gatggaagac tggctgacag cgaagctcgc atacgaaggt
cttacgtcaa aaacctacct ttcaaagacg ctggcgcaat atacgtcaaa aacatgctcc aactgcgggt ttactataac gactgccgat
tatgacggga tgttggtaag gcttaaaaag acttctgatg gatgggcaac taccctcaac aacaaagaat taaaagccga aggccagata
acgtattata accggtataa aaggcaaacc gtggaaaaag aactctccgc agagcttgac aggctttcag aagagtcggg caataatgat
atttctaagt ggaccaaggg tcgccgggac gaggcattat ttttgttaaa gaaaagattc agccatcggc ctgttcagga acagtttgtt
tgcctcgatt gcggccatga agtccacgcc gatgaacagg cagccttgaa tattgcaagg tcatggcttt ttctaaactc aaattcaaca
gaattcaaaa gttataaatc gggtaaacag cccttcgttg gtgcttggca ggccttttac aaaaggaggc ttaaagaggt atggaagccc
aacgcctgat

The gene editor effector can also be CasY.1-CasY.6, examples of which are shown in FIG. 2. CasY.1-CasY.6 has TA PAM, and a shorter PAM sequence can be useful as there are less targeting limitations. The size of CasY.1-CasY.6 (1125 bp) provides the potential for two gRNA plus one siRNA or four gRNA in a delivery plasmid. CasY.1-CasY.6 can be derived from phyla radiation (CPR) bacteria, such as, but not limited to, katanobacteria, vogelbacteria, parcubacteria, komeilibacteria, or kerfeldbacteria The sequences for CasY.1-CasY.6 are below.

CasY.1 Candidatus katanobacteria amino acid sequence 1125 aa
(SEQ ID NO: 9):
MRKKLFKGYILHNKRLVYTGKAAIRSIKYPLVAPNKTALNNLSEKIIYDYEHLFGPLNVASYARNSNRYSLVDF
WIDSLRAGVIWQSKSTSLIDLISKLEGSKSPSEKIFEQIDFELKNKLDKEQFKDIILLNTGIRSSSNVRSLRGRFLKCFKEEFRD
TEEVIACVDKWSKDLIVEGKSILVSKQFLYWEEEFGIKIFPHFKDNHDLPKLTFFVEPSLEFSPHLPLANCLERLKKFDISRES
LLGLDNNFSAFSNYFNELFNLLSRGEIKKIVTAVLAVSKSWENEPELEKRLHFLSEKAKLLGYPKLTSSWADYRMIIGGKIKS
WHSNYTEQLIKVREDLKKHQIALDKLQEDLKKVVDSSLREQIEAQREALLPLLDTMLKEKDFSDDLELYRFILSDFKSLLNG
SYQRYIQTEEERKEDRDVTKKYKDLYSNLRNIPRFFGESKKEQFNKFINKSLPTIDVGLKILEDIRNALETVSVRKPPSITEEY
VTKQLEKLSRKYKINAFNSNRFKQITEQVLRKYNNGELPKISEVFYRYPRESHVAIRILPVKISNPRKDISYLLDKYQISPDWK
NSNPGEVVDLIEIYKLTLGWLLSCNKDFSMDFSSYDLKLFPEAASLIKNFGSCLSGYYLSKMIFNCITSEIKGMITLYTRDKF
VVRYVTQMIGSNQKFPLLCLVGEKQTKNFSRNWGVLIEEKGDLGEEKNQEKCLIFKDKTDFAKAKEVEIFKNNIWRIRTS
KYQIQFLNRLFKKTKEWDLMNLVLSEPSLVLEEEWGVSWDKDKLLPLLKKEKSCEERLYYSLPLNLVPATDYKEQSAEIEQ
RNTYLGLDVGEFGVAYAVVRIVRDRIELLSWGFLKDPALRKIRERVQDMKKKQVMAVFSSSSTAVARVREMAIHSLRN
QIHSIALAYKAKIIYEISISNFETGGNRMAKIYRSIKVSDVYRESGADTLVSEMIWGKKNKQMGNHISSYATSYTCCNCART
PFELVIDNDKEYEKGGDEFIFNVGDEKKVRGFLQKSLLGKTIKGKEVLKSIKEYARPPIREVLLEGEDVEQLLKRRGNSYIYR
CPFCGYKTDADIQAALNIACRGYISDNAKDAVKEGERKLDYILEVRKLWEKNGAVLRSAKFL
CasY.1 Candidatus katanobacteria nucleic acid sequence
(SEQ ID NO: 10):
at gcgcaaaaaa ttgtttaagg gttacatttt acataataag aggcttgtat atacaggtaa agctgcaata cgttctatta
aatatccatt agtcgctcca aataaaacag ccttaaacaa tttatcagaa aagataattt atgattatga gcatttattc ggacctttaa
atgtggctag ctatgcaaga aattcaaaca ggtacagcct tgtggatttt tggatagata gcttgcgagc aggtgtaatt tggcaaagca
aaagtacttc gctaattgat ttgataagta agctagaagg atctaaatcc ccatcagaaa agatatttga acaaatagat tttgagctaa
aaaataagtt ggataaagag caattcaaag atattattct tcttaataca ggaattcgtt ctagcagtaa tgttcgcagt ttgagggggc
gctttctaaa gtgttttaaa gaggaattta gagataccga agaggttatc gcctgtgtag ataaatggag caaggacctt atcgtagagg
gtaaaagtat actagtgagt aaacagtttc tttattggga agaagagttt ggtattaaaa tttttcctca ttttaaagat aatcacgatt
taccaaaact aacttttttt gtggagcctt ccttggaatt tagtccgcac ctccctttag ccaactgtct tgagcgtttg aaaaaattcg
atatttcgcg tgaaagtttg ctcgggttag acaataattt ttcggccttt tctaattatt tcaatgagct ttttaactta ttgtccaggg
gggagattaa aaagattgta acagctgtcc ttgctgtttc taaatcgtgg gagaatgagc cagaattgga aaagcgctta cattttttga
gtgagaaggc aaagttatta gggtacccta agcttacttc ttcgtgggcg gattatagaa tgattattgg cggaaaaatt aaatcttggc
attctaacta taccgaacaa ttaataaaag ttagagagga cttaaagaaa catcaaatcg cccttgataa attacaggaa gatttaaaaa
aagtagtaga tagctcttta agagaacaaa tagaagctca acgagaagct ttgcttcctt tgcttgatac catgttaaaa gaaaaagatt
tttccgatga tttagagctt tacagattta tcttgtcaga ttttaagagt ttgttaaatg ggtcttatca aagatatatt caaacagaag
aggagagaaa ggaggacaga gatgttacca aaaaatataa agatttatat agtaatttgc gcaacatacc tagatttttt ggggaaagta
aaaaggaaca attcaataaa tttataaata aatctctccc gaccatagat gttggtttaa aaatacttga ggatattcgt aatgctctag
aaactgtaag tgttcgcaaa cccccttcaa taacagaaga gtatgtaaca aagcaacttg agaagttaag tagaaagtac aaaattaacg
cctttaattc aaacagattt aaacaaataa ctgaacaggt gctcagaaaa tataataacg gagaactacc aaagatctcg gaggtttttt
atagataccc gagagaatct catgtggcta taagaatatt acctgttaaa ataagcaatc caagaaagga tatatcttat cttctcgaca
aatatcaaat tagccccgac tggaaaaaca gtaacccagg agaagttgta gatttgatag agatatataa attgacattg ggttggctct
tgagttgtaa caaggatttt tcgatggatt tttcatcgta tgacttgaaa ctcttcccag aagccgcttc cctcataaaa aattttggct
cttgcttgag tggttactat ttaagcaaaa tgatatttaa ttgcataacc agtgaaataa aggggatgat tactttatat actagagaca
agtttgttgt tagatatgtt acacaaatga taggtagcaa tcagaaattt cctttgttat gtttggtggg agagaaacag actaaaaact
tttctcgcaa ctggggtgta ttgatagaag agaagggaga tttgggggag gaaaaaaacc aggaaaaatg tttgatattt aaggataaaa
cagattttgc taaagctaaa gaagtagaaa tttttaaaaa taatatttgg cgtatcagaa cctctaagta ccaaatccaa tttttgaata
ggctttttaa gaaaaccaaa gaatgggatt taatgaatct tgtattgagc gagcctagct tagtattgga ggaggaatgg ggtgtttcgt
gggataaaga taaactttta cctttactga agaaagaaaa atcttgcgaa gaaagattat attactcact tccccttaac ttggtgcctg
ccacagatta taaggagcaa tctgcagaaa tagagcaaag gaatacatat ttgggtttgg atgttggaga atttggtgtt gcctatgcag
tggtaagaat agtaagggac agaatagagc ttctgtcctg gggattcctt aaggacccag ctcttcgaaa aataagagag cgtgtacagg
atatgaagaa aaagcaggta atggcagtat tttctagctc ttccacagct gtcgcgcgag tacgagaaat ggctatacac tctttaagaa
atcaaattca tagcattgct ttggcgtata aagcaaagat aatttatgag atatctataa gcaattttga gacaggtggt aatagaatgg
ctaaaatata ccgatctata aaggtttcag atgtttatag ggagagtggt gcggataccc tagtttcaga gatgatctgg ggcaaaaaga
ataagcaaat gggaaaccat atatcttcct atgcgacaag ttacacttgt tgcaattgtg caagaacccc ttttgaactt gttatagata
atgacaagga atatgaaaag ggaggcgacg aatttatttt taatgttggc gatgaaaaga aggtaagggg gtttttacaa aagagtctgt
taggaaaaac aattaaaggg aaggaagtgt tgaagtctat aaaagagtac gcaaggccgc ctataaggga agtcttgctt gaaggagaag
atgtagagca gttgttgaag aggagaggaa atagctatat ttatagatgc cctttttgtg gatataaaac tgatgcggat attcaagcgg
cgttgaatat agcttgtagg ggatatattt cggataacgc aaaggatgct gtgaaggaag gagaaagaaa attagattac attttggaag
ttagaaaatt gtgggagaag aatggagctg ttttgagaag cgccaaattt ttatagtt
CasY.2 Candidatus vogelbacteria amino acid sequence 1226 aa
(SEQ ID NO: 11):
MQKVRKTLSEVHKNPYGTKVRNAKTGYSLQIERLSYTGKEGMRSFKIPLENKNKEVFDEFVKKIRNDYISQV
GLLNLSDWYEHYQEKQEHYSLADFWLDSLRAGVIFAHKETEIKNLISKIRGDKSIVDKFNASIKKKHADLYALVDIKALYDF
LTSDARRGLKTEEEFFNSKRNTLFPKFRKKDNKAVDLWVKKFIGLDNKDKLNFTKKFIGFDPNPQIKYDHTFFFHQDINF
DLERITTPKELISTYKKFLGKNKDLYGSDETTEDQLKMVLGFHNNHGAFSKYFNASLEAFRGRDNSLVEQIINNSPYWNS
HRKELEKRIIFLQVQSKKIKETELGKPHEYLASFGGKFESWVSNYLRQEEEVKRQLFGYEENKKGQKKFIVGNKQELDKIIR
GTDEYEIKAISKETIGLTQKCLKLLEQLKDSVDDYTLSLYRQLIVELRIRLNVEFQETYPELIGKSEKDKEKDAKNKRADKRYP
QIFKDIKLIPNFLGETKQMVYKKFIRSADILYEGINFIDQIDKQITQNLLPCFKNDKERIEFTEKQFETLRRKYYLMNSSRFHH
VIEGIINNRKLIEMKKRENSELKTFSDSKFVLSKLFLKKGKKYENEVYYTFYINPKARDQRRIKIVLDINGNNSVGILQDLVQ
KLKPKWDDIIKKNDMGELIDAIEIEKVRLGILIALYCEHKFKIKKELLSLDLFASAYQYLELEDDPEELSGTNLGRFLQSLVCSE
IKGAINKISRTEYIERYTVQPMNTEKNYPLLINKEGKATWHIAAKDDLSKKKGGGTVAMNQKIGKNFFGKQDYKTVFML
QDKRFDLLTSKYHLQFLSKTLDTGGGSWWKNKNIDLNLSSYSFIFEQKVKVEWDLTNLDHPIKIKPSENSDDRRLFVSIPF
VIKPKQTKRKDLQTRVNYMGIDIGEYGLAWTIINIDLKNKKINKISKQGFIYEPLTHKVRDYVATIKDNQVRGTFGMPDTK
LARLRENAITSLRNQVHDIAMRYDAKPVYEFEISNFETGSNKVKVIYDSVKRADIGRGQNNTEADNTEVNLVWGKTSKQ
FGSQIGAYATSYICSFCGYSPYYEFENSKSGDEEGARDNLYQMKKLSRPSLEDFLQGNPVYKTFRDFDKYKNDQRLQKTG
DKDGEWKTHRGNTAIYACQKCRHISDADIQASYWIALKQVVRDFYKDKEMDGDLIQGDNKDKRKVNELNRLIGVHKD
VPIINKNLITSLDINLL
CasY.2 Candidatus vogelbacteria nucleic acid sequence
(SEQ ID NO: 12):
a tggtattagg ttttcataat aatcacggcg ctttttctaa gtatttcaac gcgagcttgg aagcttttag ggggagagac
aactccttgg ttgaacaaat aattaataat tctccttact ggaatagcca tcggaaagaa ttggaaaaga gaatcatttt tttgcaagtt
cagtctaaaa aaataaaaga gaccgaactg ggaaagcctc acgagtatct tgcgagtttt ggcgggaagt ttgaatcttg ggtttcaaac
tatttacgtc aggaagaaga ggtcaaacgt caactttttg gttatgagga gaataaaaaa ggccagaaaa aatttatcgt gggcaacaaa
caagagctag ataaaatcat cagagggaca gatgagtatg agattaaagc gatttctaag gaaaccattg gacttactca gaaatgttta
aaattacttg aacaactaaa agatagtgtc gatgattata cacttagcct atatcggcaa ctcatagtcg aattgagaat cagactgaat
gttgaattcc aagaaactta tccggaatta atcggtaaga gtgagaaaga taaagaaaaa gatgcgaaaa ataaacgggc agacaagcgt
tacccgcaaa tttttaagga tataaaatta atccccaatt ttctcggtga aacgaaacaa atggtatata agaaatttat tcgttccgct
gacatccttt atgaaggaat aaattttatc gaccagatcg ataaacagat tactcaaaat ttgttgcctt gttttaagaa cgacaaggaa
cggattgaat ttaccgaaaa acaatttgaa actttacggc gaaaatacta tctgatgaat agttcccgtt ttcaccatgt tattgaagga
ataatcaata ataggaaact tattgaaatg aaaaagagag aaaatagcga gttgaaaact ttctccgata gtaagtttgt tttatctaag
ctttttctta aaaaaggcaa aaaatatgaa aatgaggtct attatacttt ttatataaat ccgaaagctc gtgaccagcg acggataaaa
attgttcttg atataaatgg gaacaattca gtcggaattt tacaagatct tgtccaaaag ttgaaaccaa aatgggacga catcataaag
aaaaatgata tgggagaatt aatcgatgca atcgagattg agaaagtccg gctcggcatc ttgatagcgt tatactgtga gcataaattc
aaaattaaaa aagaactctt gtcattagat ttgtttgcca gtgcctatca atatctagaa ttggaagatg accctgaaga actttctggg
acaaacctag gtcggttttt acaatccttg gtctgctccg aaattaaagg tgcgattaat aaaataagca ggacagaata tatagagcgg
tatactgtcc agccgatgaa tacggagaaa aactatcctt tactcatcaa taaggaggga aaagccactt ggcatattgc tgctaaggat
gacttgtcca agaagaaggg tgggggcact gtcgctatga atcaaaaaat cggcaagaat ttttttggga aacaagatta taaaactgtg
tttatgcttc aggataagcg gtttgatcta ctaacctcaa agtatcactt gcagttttta tctaaaactc ttgatactgg tggagggtct
tggtggaaaa acaaaaatat tgatttaaat ttaagctctt attctttcat tttcgaacaa aaagtaaaag tcgaatggga tttaaccaat
cttgaccatc ctataaagat taagcctagc gagaacagtg atgatagaag gcttttcgta tccattcctt ttgttattaa accgaaacag
acaaaaagaa aggatttgca aactcgagtc aattatatgg ggattgatat cggagaatat ggtttggctt ggacaattat taatattgat
ttaaagaata aaaaaataaa taagatttca aaacaaggtt tcatctatga gccgttgaca cataaagtgc gcgattatgt tgctaccatt
aaagataatc aggttagagg aacttttggc atgcctgata cgaaactagc cagattgcga gaaaatgcca ttaccagctt gcgcaatcaa
gtgcatgata ttgctatgcg ctatgacgcc aaaccggtat atgaatttga aatttccaat tttgaaacgg ggtctaataa agtgaaagta
atttatgatt cggttaagcg agctgatatc ggccgaggcc agaataatac cgaagcagac aatactgagg ttaatcttgt ctgggggaag
acaagcaaac aatttggcag tcaaatcggc gcttatgcga caagttacat ctgttcattt tgtggttatt ctccatatta tgaatttgaa
aattctaagt cgggagatga agaaggggct agagataatc tatatcagat gaagaaattg agtcgcccct ctcttgaaga tttcctccaa
ggaaatccgg tttataagac atttagggat tttgataagt ataaaaacga tcaacggttg caaaagacgg gtgataaaga tggtgaatgg
aaaacacaca gagggaatac tgcaatatac gcctgtcaaa agtgtagaca tatctctgat gcggatatcc aagcatcata ttggattgct
ttgaagcaag ttgtaagaga tttttataaa gacaaagaga tggatggtga tttgattcaa ggagataata aagacaagag aaaagtaaac
gagcttaata gacttattgg agtacataaa gatgtgccta taataaataa aaatttaata acatcactcg acataaactt actataga
CasY.3 Candidatus vogelbacteria amino acid sequence 1200 aa
(SEQ ID NO: 13):
MKAKKSFYNQKRKFGKRGYRLHDERIAYSGGIGSMRSIKYELKDSYGIAGLRNRIADATISDNKWLYGNINLN
DYLEWRSSKTDKQIEDGDRESSLLGFWLEALRLGFVFSKQSHAPNDFNETALQDLFETLDDDLKHVLDRKKWCDFIKIGT
PKTNDQGRLKKQIKNLLKGNKREEIEKTLNESDDELKEKINRIADVFAKNKSDKYTIFKLDKPNTEKYPRINDVQVAFFCHP
DFEEITERDRTKTLDLIINRFNKRYEITENKKDDKTSNRMALYSLNQGYIPRVLNDLFLFVKDNEDDFSQFLSDLENFFSFS
NEQIKIIKERLKKLKKYAEPIPGKPQLADKWDDYASDFGGKLESWYSNRIEKLKKIPESVSDLRNNLEKIRNVLKKQNNASK
ILELSQKIIEYIRDYGVSFEKPEIIKFSWINKTKDGQKKVFYVAKMADREFIEKLDLWMADLRSQLNEYNQDNKVSFKKKG
KKIEELGVLDFALNKAKKNKSTKNENGWQQKLSESIQSAPLFFGEGNRVRNEEVYNLKDLLFSEIKNVENILMSSEAEDLK
NIKIEYKEDGAKKGNYVLNVLARFYARFNEDGYGGWNKVKTVLENIAREAGTDFSKYGNNNNRNAGRFYLNGRERQV
FTLIKFEKSITVEKILELVKLPSLLDEAYRDLVNENKNHKLRDVIQLSKTIMALVLSHSDKEKQIGGNYIHSKLSGYNALISKR
DFISRYSVQTTNGTQCKLAIGKGKSKKGNEIDRYFYAFQFFKNDDSKINLKVIKNNSHKNIDFNDNENKINALQVYSSNY
QIQFLDWFFEKHQGKKTSLEVGGSFTIAEKSLTIDWSGSNPRVGFKRSDTEEKRVFVSQPFTLIPDDEDKERRKERMIKTK
NRFIGIDIGEYGLAWSLIEVDNGDKNNRGIRQLESGFITDNQQQVLKKNVKSWRQNQIRQTFTSPDTKIARLRESLIGSY
KNQLESLMVAKKANLSFEYEVSGFEVGGKRVAKIYDSIKRGSVRKKDNNSQNDQSWGKKGINEWSFETTAAGTSQFCT
HCKRWSSLAIVDIEEYELKDYNDNLFKVKINDGEVRLLGKKGWRSGEKIKGKELFGPVKDAMRPNVDGLGMKIVKRKYL
KLDLRDWVSRYGNMAIFICPYVDCHHISHADKQAAFNIAVRGYLKSVNPDRAIKHGDKGLSRDFLCQEEGKLNFEQIGL
L
CasY.3 Candidatus vogelbacteria nucleic acid sequence
(SEQ ID NO: 14):
atgaaa gctaaaaaaa gtttttataa tcaaaagcgg aagttcggta aaagaggtta tcgtcttcac gatgaacgta
tcgcgtattc aggagggatt ggatcgatgc gatctattaa atatgaattg aaggattcgt atggaattgc tgggcttcgt aatcgaatcg
ctgacgcaac tatttctgat aataagtggc tgtacgggaa tataaatcta aatgattatt tagagtggcg atcttcaaag actgacaaac
agattgaaga cggagaccga gaatcatcac tcctgggttt ttggctggaa gcgttacgac tgggattcgt gttttcaaaa caatctcatg
ctccgaatga ttttaacgag accgctctac aagatttgtt tgaaactctt gatgatgatt tgaaacatgt tcttgatagg aaaaaatggt
gtgactttat caagatagga acacctaaga caaatgacca aggtcgttta aaaaaacaaa tcaagaattt gttaaaagga aacaagagag
aggaaattga aaaaactctc aatgaatcag acgatgaatt gaaagagaaa ataaacagaa ttgccgatgt ttttgcaaaa aataagtctg
ataaatacac aattttcaaa ttagataaac ccaatacgga aaaatacccc agaatcaacg atgttcaggt ggcgtttttt tgtcatcccg
attttgagga aattacagaa cgagatagaa caaagactct agatctgatc attaatcggt ttaataagag atatgaaatt accgaaaata
aaaaagatga caaaacttca aacaggatgg ccttgtattc cttgaaccag ggctatattc ctcgcgtcct gaatgattta ttcttgtttg
tcaaagacaa tgaggatgat tttagtcagt ttttatctga tttggagaat ttcttctctt tttccaacga acaaattaaa ataataaagg
aaaggttaaa aaaacttaaa aaatatgctg aaccaattcc cggaaagccg caacttgctg ataaatggga cgattatgct tctgattttg
gcggtaaatt ggaaagctgg tactccaatc gaatagagaa attaaagaag attccggaaa gcgtttccga tctgcggaat aatttggaaa
agatacgcaa tgttttaaaa aaacaaaata atgcatctaa aatcctggag ttatctcaaa agatcattga atacatcaga gattatggag
tttcttttga aaagccggag ataattaagt tcagctggat aaataagacg aaggatggtc agaaaaaagt tttctatgtt gcgaaaatgg
cggatagaga attcatagaa aagcttgatt tatggatggc tgatttacgc agtcaattaa atgaatacaa tcaagataat aaagtttctt
tcaaaaagaa aggtaaaaaa atagaagagc tcggtgtctt ggattttgct cttaataaag cgaaaaaaaa taaaagtaca aaaaatgaaa
atggctggca acaaaaattg tcagaatcta ttcaatctgc cccgttattt tttggcgaag ggaatcgtgt acgaaatgaa gaagtttata
atttgaagga ccttctgttt tcagaaatca agaatgttga aaatatttta atgagctcgg aagcggaaga cttaaaaaat ataaaaattg
aatataaaga agatggcgcg aaaaaaggga actatgtctt gaatgtcttg gctagatttt acgcgagatt caatgaggat ggctatggtg
gttggaacaa agtaaaaacc gttttggaaa atattgcccg agaggcgggg actgattttt caaaatatgg aaataataac aatagaaatg
ccggcagatt ttatctaaac ggccgcgaac gacaagtttt tactctaatc aagtttgaaa aaagtatcac ggtggaaaaa atacttgaat
tggtaaaatt acctagccta cttgatgaag cgtatagaga tttagtcaac gaaaataaaa atcataaatt acgcgacgta attcaattga
gcaagacaat tatggctctg gttttatctc attctgataa agaaaaacaa attggaggaa attatatcca tagtaaattg agcggataca
atgcgcttat ttcaaagcga gattttatct cgcggtatag cgtgcaaacg accaacggaa ctcaatgtaa attagccata ggaaaaggca
aaagcaaaaa aggtaatgaa attgacaggt atttctacgc ttttcaattt tttaagaatg acgacagcaa aattaattta aaggtaatca
aaaataattc gcataaaaac atcgatttca acgacaatga aaataaaatt aacgcattgc aagtgtattc atcaaactat cagattcaat
tcttagactg gttttttgaa aaacatcaag ggaagaaaac atcgctcgag gtcggcggat cttttaccat cgccgaaaag agtttgacaa
tagactggtc ggggagtaat ccgagagtcg gttttaaaag aagcgacacg gaagaaaaga gggtttttgt ctcgcaacca tttacattaa
taccagacga tgaagacaaa gagcgtcgta aagaaagaat gataaagacg aaaaaccgtt ttatcggtat cgatatcggt gaatatggtc
tggcttggag tctaatcgaa gtggacaatg gagataaaaa taatagagga attagacaac ttgagagcgg ttttattaca gacaatcagc
agcaagtctt aaagaaaaac gtaaaatcct ggaggcaaaa ccaaattcgt caaacgttta cttcaccaga cacaaaaatt gctcgtcttc
gtgaaagttt gatcggaagt tacaaaaatc aactggaaag tctgatggtt gctaaaaaag caaatcttag ttttgaatac gaagtttccg
ggtttgaagt tgggggaaag agggttgcaa aaatatacga tagtataaag cgtgggtcgg tgcgtaaaaa ggataataac tcacaaaatg
atcaaagttg gggtaaaaag ggaattaatg agtggtcatt cgagacgacg gctgccggaa catcgcaatt ttgtactcat tgcaagcggt
ggagcagttt agcgatagta gatattgaag aatatgaatt aaaagattac aacgataatt tatttaaggt aaaaattaat gatggtgaag
ttcgtctcct tggtaagaaa ggttggagat ccggcgaaaa gatcaaaggg aaagaattat ttggtcccgt caaagacgca atgcgcccaa
atgttgacgg actagggatg aaaattgtaa aaagaaaata tctaaaactt gatctccgcg attgggtttc aagatatggg aatatggcta
ttttcatctg tccttatgtc gattgccacc atatctctca tgcggataaa caagctgctt ttaatattgc cgtgcgaggg tatttgaaaa
gcgttaatcc tgacagagca ataaaacacg gagataaagg tttgtctagg gactttttgt gccaagaaga gggtaagctt aattttgaac
aaatagggtt attatgaa
CasY.4 Candidatus parcubacteria amino acid sequence 1210 aa
(SEQ ID NO: 15):
MSKRHPRISGVKGYRLHAQRLEYTGKSGAMRTIKYPLYSSPSGGRTVPREIVSAINDDYVGLYGLSNFDDLYN
AEKRNEEKVYSVLDFWYDCVQYGAVFSYTAPGLLKNVAEVRGGSYELTKTLKGSHLYDELQIDKVIKFLNKKEISRANGSL
DKLKKDIIDCFKAEYRERHKDQCNKLADDIKNAKKDAGASLGERQKKLFRDFFGISEQSENDKPSFTNPLNLTCCLLPFDT
VNNNRNRGEVLFNKLKEYAQKLDKNEGSLEMWEYIGIGNSGTAFSNFLGEGFLGRLRENKITELKKAMMDITDAWRG
QEQEEELEKRLRILAALTIKLREPKFDNHWGGYRSDINGKLSSWLQNYINQTVKIKEDLKGHKKDLKKAKEMINRFGESD
TKEEAVVSSLLESIEKIVPDDSADDEKPDIPAIAIYRRFLSDGRLTLNRFVQREDVQEALIKERLEAEKKKKPKKRKKKSDAE
DEKETIDFKELFPHLAKPLKLVPNFYGDSKRELYKKYKNAAIYTDALWKAVEKIYKSAFSSSLKNSFFDTDFDKDFFIKRLQK
IFSVYRRFNTDKWKPIVKNSFAPYCDIVSLAENEVLYKPKQSRSRKSAAIDKNRVRLPSTENIAKAGIALARELSVAGFDW
KDLLKKEEHEEYIDLIELHKTALALLLAVTETQLDISALDFVENGTVKDFMKTRDGNLVLEGRFLEMFSQSIVFSELRGLAG
LMSRKEFITRSAIQTMNGKQAELLYIPHEFQSAKITTPKEMSRAFLDLAPAEFATSLEPESLSEKSLLKLKQMRYYPHYFGY
ELTRTGQGIDGGVAENALRLEKSPVKKREIKCKQYKTLGRGQNKIVLYVRSSYYQTQFLEWFLHRPKNVQTDVAVSGSFL
IDEKKVKTRWNYDALTVALEPVSGSERVFVSQPFTIFPEKSAEEEGQRYLGIDIGEYGIAYTALEITGDSAKILDQNFISDPQ
LKTLREEVKGLKLDQRRGTFAMPSTKIARIRESLVHSLRNRIHHLALKHKAKIVYELEVSRFEEGKQKIKKVYATLKKADVYS
EIDADKNLQTTVWGKLAVASEISASYTSQFCGACKKLWRAEMQVDETITTQELIGTVRVIKGGTLIDAIKDFMRPPIFDE
NDTPFPKYRDFCDKHHISKKMRGNSCLFICPFCRANADADIQASQTIALLRYVKEEKKVEDYFERFRKLKNIKVLGQMKKI
CasY.4 Candidatus parcubacteria nucleic acid sequence
(SEQ ID NO: 16):
atgagtaagc gacatcctag aattagcggc gtaaaagggt accgtttgca tgcgcaacgg ctggaatata ccggcaaaag
tggggcaatg cgaacgatta aatatcctct ttattcatct ccgagcggtg gaagaacggt tccgcgcgag atagtttcag caatcaatga
tgattatgta gggctgtacg gtttgagtaa ttttgacgat ctgtataatg cggaaaagcg caacgaagaa aaggtctact cggttttaga
tttttggtac gactgcgtcc aatacggcgc ggttttttcg tatacagcgc cgggtctttt gaaaaatgtt gccgaagttc gcgggggaag
ctacgaactt acaaaaacgc ttaaagggag ccatttatat gatgaattgc aaattgataa agtaattaaa tttttgaata aaaaagaaat
ttcgcgagca aacggatcgc ttgataaact gaagaaagac atcattgatt gcttcaaagc agaatatcgg gaacgacata aagatcaatg
caataaactg gctgatgata ttaaaaatgc aaaaaaagac gcgggagctt ctttagggga gcgtcaaaaa aaattatttc gcgatttttt
tggaatttca gagcagtctg aaaatgataa accgtctttt actaatccgc taaacttaac ctgctgttta ttgccttttg acacagtgaa
taacaacaga aaccgcggcg aagttttgtt taacaagctc aaggaatatg ctcaaaaatt ggataaaaac gaagggtcgc ttgaaatgtg
ggaatatatt ggcatcggga acagcggcac tgccttttct aattttttag gagaagggtt tttgggcaga ttgcgcgaga ataaaattac
agagctgaaa aaagccatga tggatattac agatgcatgg cgtgggcagg aacaggaaga agagttagaa aaacgtctgc ggatacttgc
cgcgcttacc ataaaattgc gcgagccgaa atttgacaac cactggggag ggtatcgcag tgatataaac ggcaaattat ctagctggct
tcagaattac ataaatcaaa cagtcaaaat caaagaggac ttaaagggac acaaaaagga cctgaaaaaa gcgaaagaga tgataaatag
gtttggggaa agcgacacaa aggaagaggc ggttgtttca tctttgcttg aaagcattga aaaaattgtt cctgatgata gcgctgatga
cgagaaaccc gatattccag ctattgctat ctatcgccgc tttctttcgg atggacgatt aacattgaat cgctttgtcc aaagagaaga
tgtgcaagag gcgctgataa aagaaagatt ggaagcggag aaaaagaaaa aaccgaaaaa gcgaaaaaag aaaagtgacg
ctgaagatga aaaagaaaca attgacttca aggagttatt tcctcatctt gccaaaccat taaaattggt gccaaacttt tacggcgaca
gtaagcgtga gctgtacaag aaatataaga acgccgctat ttatacagat gctctgtgga aagcagtgga aaaaatatac aaaagcgcgt
tctcgtcgtc tctaaaaaat tcattttttg atacagattt tgataaagat ttttttatta agcggcttca gaaaattttt tcggtttatc
gtcggtttaa tacagacaaa tggaaaccga ttgtgaaaaa ctctttcgcg ccctattgcg acatcgtctc acttgcggag aatgaagttt
tgtataaacc gaaacagtcg cgcagtagaa aatctgccgc gattgataaa aacagagtgc gtctcccttc cactgaaaat atcgcaaaag
ctggcattgc cctcgcgcgg gagctttcag tcgcaggatt tgactggaaa gatttgttaa aaaaagagga gcatgaagaa tacattgatc
tcatagaatt gcacaaaacc gcgcttgcgc ttcttcttgc cgtaacagaa acacagcttg acataagcgc gttggatttt gtagaaaatg
ggacggtcaa ggattttatg aaaacgcggg acggcaatct ggttttggaa gggcgtttcc ttgaaatgtt ctcgcagtca attgtgtttt
cagaattgcg cgggcttgcg ggtttaatga gccgcaagga atttatcact cgctccgcga ttcaaactat gaacggcaaa caggcggagc
ttctctacat tccgcatgaa ttccaatcgg caaaaattac aacgccaaag gaaatgagca gggcgtttct tgaccttgcg cccgcggaat
ttgctacatc gcttgagcca gaatcgcttt cggagaagtc attattgaaa ttgaagcaga tgcggtacta tccgcattat tttggatatg
agcttacgcg aacaggacag gggattgatg gtggagtcgc ggaaaatgcg ttacgacttg agaagtcgcc agtaaaaaaa cgagagataa
aatgcaaaca gtataaaact ttgggacgcg gacaaaataa aatagtgtta tatgtccgca gttcttatta tcagacgcaa tttttggaat
ggtttttgca tcggccgaaa aacgttcaaa ccgatgttgc ggttagcggt tcgtttctta tcgacgaaaa gaaagtaaaa actcgctgga
attatgacgc gcttacagtc gcgcttgaac cagtttccgg aagcgagcgg gtctttgtct cacagccgtt tactattttt ccggaaaaaa
gcgcagagga agaaggacag aggtatcttg gcatagacat cggcgaatac ggcattgcgt atactgcgct tgagataact ggcgacagtg
caaagattct tgatcaaaat tttatttcag acccccagct taaaactctg cgcgaggagg tcaaaggatt aaaacttgac caaaggcgcg
ggacatttgc catgccaagc acgaaaatcg cccgcatccg cgaaagcctt gtgcatagtt tgcggaaccg catacatcat cttgcgttaa
agcacaaagc aaagattgtg tatgaattgg aagtgtcgcg ttttgaagag ggaaagcaaa aaattaagaa agtctacgct acgttaaaaa
aagcggatgt gtattcagaa attgacgcgg ataaaaattt acaaacgaca gtatggggaa aattggccgt tgcaagcgaa atcagcgcaa
gctatacaag ccagttttgt ggtgcgtgta aaaaattgtg gcgggcggaa atgcaggttg acgaaacaat tacaacccaa gaactaatcg
gcacagttag agtcataaaa gggggcactc ttattgacgc gataaaggat tttatgcgcc cgccgatttt tgacgaaaat gacactccat
ttccaaaata tagagacttt tgcgacaagc atcacatttc caaaaaaatg cgtggaaaca gctgtttgtt catttgtcca ttctgccgcg
caaacgcgga tgctgatatt caagcaagcc aaacaattgc gcttttaagg tatgttaagg aagagaaaaa ggtagaggac tactttgaac
gatttagaaa gctaaaaaac attaaagtgc tcggacagat gaagaaaata tgatag
CasY.5 Candidatus komeilibacteria amino acid sequence 1192 aa
(SEQ ID NO: 17):
MAESKQMQCRKCGASMKYEVIGLGKKSCRYMCPDCGNHTSARKIQNKKKRDKKYGSASKAQSQRIAVA
GALYPDKKVQTIKTYKYPADLNGEVHDRGVAEKIEQAIQEDEIGLLGPSSEYACWIASQKQSEPYSVVDFWFDAVCAGG
VFAYSGARLLSTVLQLSGEESVLRAALASSPFVDDINLAQAEKFLAVSRRTGQDKLGKRIGECFAEGRLEALGIKDRMREF
VQAIDVAQTAGQRFAAKLKIFGISQMPEAKQWNNDSGLTVCILPDYYVPEENRADQLVVLLRRLREIAYCMGIEDEAGF
EHLGIDPGALSNFSNGNPKRGFLGRLLNNDIIALANNMSAMTPYWEGRKGELIERLAWLKHRAEGLYLKEPHFGNSWA
DHRSRIFSRIAGWLSGCAGKLKIAKDQISGVRTDLFLLKRLLDAVPQSAPSPDFIASISALDRFLEAAESSQDPAEQVRALY
AFHLNAPAVRSIANKAVQRSDSQEWLIKELDAVDHLEFNKAFPFFSDTGKKKKKGANSNGAPSEEEYTETESIQQPEDA
EQEVNGQEGNGASKNQKKFQRIPRFFGEGSRSEYRILTEAPQYFDMFCNNMRAIFMQLESQPRKAPRDFKCFLQNRL
QKLYKQTFLNARSNKCRALLESVLISWGEFYTYGANEKKFRLRHEASERSSDPDYVVQQALEIARRLFLFGFEWRDCSAG
ERVDLVEIHKKAISFLLAITQAEVSVGSYNWLGNSTVSRYLSVAGTDTLYGTQLEEFLNATVLSQMRGLAIRLSSQELKDG
FDVQLESSCQDNLQHLLVYRASRDLAACKRATCPAELDPKILVLPAGAFIASVMKMIERGDEPLAGAYLRHRPHSFGWQ
IRVRGVAEVGMDQGTALAFQKPTESEPFKIKPFSAQYGPVLWLNSSSYSQSQYLDGFLSQPKNWSMRVLPQAGSVRV
EQRVALIWNLQAGKMRLERSGARAFFMPVPFSFRPSGSGDEAVLAPNRYLGLFPHSGGIEYAVVDVLDSAGFKILERGT
IAVNGFSQKRGERQEEAHREKQRRGISDIGRKKPVQAEVDAANELHRKYTDVATRLGCRIVVQWAPQPKPGTAPTAQ
TVYARAVRTEAPRSGNQEDHARMKSSWGYTWSTYWEKRKPEDILGISTQVYWTGGIGESCPAVAVALLGHIRATSTQ
TEWEKEEVVFGRLKKFFPS
CasY.5 Candidatus komeilibacteria nucleic acid sequence
(SEQ ID NO: 18):
accaaccacc tattgcgtct ttttcgctca ttttagcaaa agtggctgtc tagacataca ggtggaaagg tgagagtaaa
gacatggcct gaatagcgtc ctcgtcctcg tctagacata caggtggaaa ggtgagagta aagaccggag cactcatcct ctcactctat
tttgtctaga catacaggtg gaaaggtgag agtaaagaca aaccgtgcca cactaaaccg atgagtctag acatacaggt ggaaaggtga
gagtaaagac tcaagtaact acctgttctt tcacaagtct agacatacag gtggaaaggt gagagtaaag actcaagtaa ctacctgttc
tttcacaagt ctagacctgc aggtggtaag gtgagagtaa agactcaagt aactacctgt tctttcacaa gtctagacct gcaggtggta
aggtgagagt aaagactttt atcctcctct ctatgcttct gagtctagac atttaggtgg aaaggtgaga gtaaagactt gtggagatcc
atgaacttcg gcagtctaga cctgcaggtg gaaaggtgag agtaaagacg tccttcacac gatcttcctc tgttagtcta ggcctgcagg
tggaaaggtg agagtaaaga cgcataagcg taattgaagc tctctccggt ccagaccttg tcgcgcttgt gttgcgacaa aggcggagtc
cgcaataagt tctttttaca atgttttttc cataaaaccg atacaatcaa gtatcggttt tgcttttttt atgaaaatat gttatgctat
gtgctcaaat aaaaatatca ataaaatagc gtttttttga taatttatcg ctaaaattat acataatcac gcaacattgc cattctcaca
caggagaaaa gtcatggcag aaagcaagca gatgcaatgc cgcaagtgcg gcgcaagcat gaagtatgaa gtaattggat tgggcaagaa
gtcatgcaga tatatgtgcc cagattgcgg caatcacacc agcgcgcgca agattcagaa caagaaaaag cgcgacaaaa agtatggatc
cgcaagcaaa gcgcagagcc agaggatagc tgtggctggc gcgctttatc cagacaaaaa agtgcagacc ataaagacct acaaataccc
agcggatctg aatggcgaag ttcatgacag aggcgtcgca gagaagattg agcaggcgat tcaggaagat gagatcggcc tgcttggccc
gtccagcgaa tacgcttgct ggattgcttc acaaaaacaa agcgagccgt attcagttgt agatttttgg tttgacgcgg tgtgcgcagg
cggagtattc gcgtattctg gcgcgcgcct gctttccaca gtcctccagt tgagtggcga ggaaagcgtt ttgcgcgctg ctttagcatc
tagcccgttt gtagatgaca ttaatttggc gcaagcggaa aagttcctag ccgttagccg gcgcacaggc caagataagc taggcaagcg
cattggagaa tgtttcgcgg aaggccggct tgaagcgctt ggcatcaaag atcgcatgcg cgaattcgtg caagcgattg atgtggccca
aaccgcgggc cagcggttcg cggccaagct aaagatattc ggcatcagtc agatgcctga agccaagcaa tggaacaatg attccgggct
cactgtatgt attttgccgg attattatgt cccggaagaa aaccgcgcgg accagctggt tgttttgctt cggcgcttac gcgagatcgc
gtattgcatg ggaattgagg atgaagcagg atttgagcat ctaggcattg accctggcgc tctttccaat ttttccaatg gcaatccaaa
gcgaggattt ctcggccgcc tgctcaataa tgacattata gcgctggcaa acaacatgtc agccatgacg ccgtattggg aaggcagaaa
aggcgagttg attgagcgcc ttgcatggct taaacatcgc gctgaaggat tgtatttgaa agagccacat ttcggcaact cctgggcaga
ccaccgcagc aggattttca gtcgcattgc gggctggctt tccggatgcg cgggcaagct caagattgcc aaggatcaga tttcaggcgt
gcgtacggat ttgtttctgc tcaagcgcct tctggatgcg gtaccgcaaa gcgcgccgtc gccggacttt attgcttcca tcagcgcgct
ggatcggttt ttggaagcgg cagaaagcag ccaggatccg gcagaacagg tacgcgcttt gtacgcgttt catctgaacg cgcctgcggt
ccgatccatc gccaacaagg cggtacagag gtctgattcc caggagtggc ttatcaagga actggatgct gtagatcacc ttgaattcaa
caaagcattt ccgttttttt cggatacagg aaagaaaaag aagaaaggag cgaatagcaa cggagcgcct tctgaagaag aatacacgga
aacagaatcc attcaacaac cagaagatgc agagcaggaa gtgaatggtc aagaaggaaa tggcgcttca aagaaccaga aaaagtttca
gcgcattcct cgatttttcg gggaagggtc aaggagtgag tatcgaattt taacagaagc gccgcaatat tttgacatgt tctgcaataa
tatgcgcgcg atctttatgc agctagagag tcagccgcgc aaggcgcctc gtgatttcaa atgctttctg cagaatcgtt tgcagaagct
ttacaagcaa acctttctca atgctcgcag taataaatgc cgcgcgcttc tggaatccgt ccttatttca tggggagaat tttatactta
tggcgcgaat gaaaagaagt ttcgtctgcg ccatgaagcg agcgagcgca gctcggatcc ggactatgtg gttcagcagg cattggaaat
cgcgcgccgg cttttcttgt tcggatttga gtggcgcgat tgctctgctg gagagcgcgt ggatttggtt gaaatccaca aaaaagcaat
ctcatttttg cttgcaatca ctcaggccga ggtttcagtt ggttcctata actggcttgg gaatagcacc gtgagccggt atctttcggt
tgctggcaca gacacattgt acggcactca actggaggag tttttgaacg ccacagtgct ttcacagatg cgtgggctgg cgattcggct
ttcatctcag gagttaaaag acggatttga tgttcagttg gagagttcgt gccaggacaa tctccagcat ctgctggtgt atcgcgcttc
gcgcgacttg gctgcgtgca aacgcgctac atgcccggct gaattggatc cgaaaattct tgttctgccg gctggtgcgt ttatcgcgag
cgtaatgaaa atgattgagc gtggcgatga accattagca ggcgcgtatt tgcgtcatcg gccgcattca ttcggctggc agatacgggt
tcgtggagtg gcggaagtag gcatggatca gggcacagcg ctagcattcc agaagccgac tgaatcagag ccgtttaaaa taaagccgtt
ttccgctcaa tacggcccag tactttggct taattcttca tcctatagcc agagccagta tctggatgga tttttaagcc agccaaagaa
ttggtctatg cgggtgctac ctcaagccgg atcagtgcgc gtggaacagc gcgttgctct gatatggaat ttgcaggcag gcaagatgcg
gctggagcgc tctggagcgc gcgcgttttt catgccagtg ccattcagct tcaggccgtc tggttcagga gatgaagcag tattggcgcc
gaatcggtac ttgggacttt ttccgcattc cggaggaata gaatacgcgg tggtggatgt attagattcc gcgggtttca aaattcttga
gcgcggtacg attgcggtaa atggcttttc ccagaagcgc ggcgaacgcc aagaggaggc acacagagaa aaacagagac gcggaatttc
tgatataggc cgcaagaagc cggtgcaagc tgaagttgac gcagccaatg aattgcaccg caaatacacc gatgttgcca ctcgtttagg
gtgcagaatt gtggttcagt gggcgcccca gccaaagccg ggcacagcgc cgaccgcgca aacagtatac gcgcgcgcag tgcggaccga
agcgccgcga tctggaaatc aagaggatca tgctcgtatg aaatcctctt ggggatatac ctggagcacc tattgggaga agcgcaaacc
agaggatatt ttgggcatct caacccaagt atactggacc ggcggtatag gcgagtcatg tcccgcagtc gcggttgcgc ttttggggca
cattagggca acatccactc aaactgaatg ggaaaaagag gaggttgtat tcggtcgact gaagaagttc tttccaagct agacgatctt
tttaaaaact gggctgctgg ctatcgtatg gtcagtagct cttatttttt tacttgatat atggtattat
CasY.6 Candidatus kerfeldbacteria amino acid sequence 1287 aa
(SEQ ID NO: 19):
MKRILNSLKVAALRLLFRGKGSELVKTVKYPLVSPVQGAVEELAEAIRHDNLHLFGQKEIVDLMEKDEGTQVYSVVDFW
LDTLRLGMFFSPSANALKITLGKFNSDQVSPFRKVLEQSPFFLAGRLKVEPAERILSVEIRKIGKRENRVENYAADVETCFI
GQLSSDEKQSIQKLANDIWDSKDHEEQRMLKADFFAIPLIKDPKAVTEEDPENETAGKQKPLELCVCLVPELYTRGFGSI
ADFLVQRLTLLRDKMSTDTAEDCLEYVGIEEEKGNGMNSLLGTFLKNLQGDGFEQIFQFMLGSYVGWQGKEDVLRERL
DLLAEKVKRLPKPKFAGEWSGHRMFLHGQLKSWSSNFFRLFNETRELLESIKSDIQHATMLISYVEEKGGYHPQLLSQYR
KLMEQLPALRTKVLDPEIEMTHMSEAVRSYIMIHKSVAGFLPDLLESLDRDKDREFLLSIFPRIPKIDKKTKEIVAWELPGE
PEEGYLFTANNLFRNFLENPKHVPRFMAERIPEDWTRLRSAPVWFDGMVKQWQKVVNQLVESPGALYQFNESFLRQ
RLQAMLTVYKRDLQTEKFLKLLADVCRPLVDFFGLGGNDIIFKSCQDPRKQWQTVIPLSVPADVYTACEGLAIRLRETLG
FEWKNLKGHEREDFLRLHQLLGNLLFWIRDAKLVVKLEDWMNNPCVQEYVEARKAIDLPLEIFGFEVPIFLNGYLFSELR
QLELLLRRKSVMTSYSVKTTGSPNRLFQLVYLPLNPSDPEKKNSNNFQERLDTPTGLSRRFLDLTLDAFAGKLLTDPVTQE
LKTMAGFYDHLFGFKLPCKLAAMSNHPGSSSKMVVLAKPKKGVASNIGFEPIPDPAHPVFRVRSSWPELKYLEGLLYLPE
DTPLTIELAETSVSCQSVSSVAFDLKNLTTILGRVGEFRVTADQPFKLTPIIPEKEESFIGKTYLGLDAGERSGVGFAIVTVD
GDGYEVQRLGVHEDTQLMALQQVASKSLKEPVFQPLRKGTFRQQERIRKSLRGCYWNFYHALMIKYRAKVVHEESVG
SSGLVGQWLRAFQKDLKKADVLPKKGGKNGVDKKKRESSAQDTLWGGAFSKKEEQQIAFEVQAAGSSQFCLKCGWW
FQLGMREVNRVQESGVVLDWNRSIVTFLIESSGEKVYGFSPQQLEKGFRPDIETFKKMVRDFMRPPMFDRKGRPAAA
YERFVLGRRHRRYRFDKVFEERFGRSALFICPRVGCGNFDHSSEQSAVVLALIGYIADKEGMSGKKLVYVRLAELMAEW
KLKKLERSRVEEQSSAQ
CasY.6 Candidatus kerfeldbacteria nucleic acid sequence
(SEQ ID NO: 20):
atgaagag aattctgaac agtctgaaag ttgctgcctt gagacttctg tttcgaggca aaggttctga attagtgaag
acagtcaaat atccattggt ttccccggtt caaggcgcgg ttgaagaact tgctgaagca attcggcacg acaacctgca cctttttggg
cagaaggaaa tagtggatct tatggagaaa gacgaaggaa cccaggtgta ttcggttgtg gatttttggt tggataccct gcgtttaggg
atgtttttct caccatcagc gaatgcgttg aaaatcacgc tgggaaaatt caattctgat caggtttcac cttttcgtaa ggttttggag
cagtcacctt tttttcttgc gggtcgcttg aaggttgaac ctgcggaaag gatactttct gttgaaatca gaaagattgg taaaagagaa
aacagagttg agaactatgc cgccgatgtg gagacatgct tcattggtca gctttcttca gatgagaaac agagtatcca gaagctggca
aatgatatct gggatagcaa ggatcatgag gaacagagaa tgttgaaggc ggattttttt gctatacctc ttataaaaga ccccaaagct
gtcacagaag aagatcctga aaatgaaacg gcgggaaaac agaaaccgct tgaattatgt gtttgtcttg ttcctgagtt gtatacccga
ggtttcggct ccattgctga ttttctggtt cagcgactta ccttgctgcg tgacaaaatg agtaccgaca cggcggaaga ttgcctcgag
tatgttggca ttgaggaaga aaaaggcaat ggaatgaatt ccttgctcgg cacttttttg aagaacctgc agggtgatgg ttttgaacag
atttttcagt ttatgcttgg gtcttatgtt ggctggcagg ggaaggaaga tgtactgcgc gaacgattgg atttgctggc cgaaaaagtc
aaaagattac caaagccaaa atttgccgga gaatggagtg gtcatcgtat gtttctccat ggtcagctga aaagctggtc gtcgaatttc
ttccgtcttt ttaatgagac gcgggaactt ctggaaagta tcaagagtga tattcaacat gccaccatgc tcattagcta tgtggaagag
aaaggaggct atcatccaca gctgttgagt cagtatcgga agttaatgga acaattaccg gcgttgcgga ctaaggtttt ggatcctgag
attgagatga cgcatatgtc cgaggctgtt cgaagttaca ttatgataca caagtctgta gcgggatttc tgccggattt actcgagtct
ttggatcgag ataaggatag ggaatttttg ctttccatct ttcctcgtat tccaaagata gataagaaga cgaaagagat cgttgcatgg
gagctaccgg gcgagccaga ggaaggctat ttgttcacag caaacaacct tttccggaat tttcttgaga atccgaaaca tgtgccacga
tttatggcag agaggattcc cgaggattgg acgcgtttgc gctcggcccc tgtgtggttt gatgggatgg tgaagcaatg gcagaaggtg
gtgaatcagt tggttgaatc tccaggcgcc ctttatcagt tcaatgaaag ttttttgcgt caaagactgc aagcaatgct tacggtctat
aagcgggatc tccagactga gaagtttctg aagctgctgg ctgatgtctg tcgtccactc gttgattttt tcggacttgg aggaaatgat
attatcttca agtcatgtca ggatccaaga aagcaatggc agactgttat tccactcagt gtcccagcgg atgtttatac agcatgtgaa
ggcttggcta ttcgtctccg cgaaactctt ggattcgaat ggaaaaatct gaaaggacac gagcgggaag attttttacg gctgcatcag
ttgctgggaa atctgctgtt ctggatcagg gatgcgaaac ttgtcgtgaa gctggaagac tggatgaaca atccttgtgt tcaggagtat
gtggaagcac gaaaagccat tgatcttccc ttggagattt tcggatttga ggtgccgatt tttctcaatg gctatctctt ttcggaactg
cgccagctgg aattgttgct gaggcgtaag tcggtgatga cgtcttacag cgtcaaaacg acaggctcgc caaataggct cttccagttg
gtttacctac ctctaaaccc ttcagatccg gaaaagaaaa attccaacaa ctttcaggag cgcctcgata cacctaccgg tttgtcgcgt
cgttttctgg atcttacgct ggatgcattt gctggcaaac tcttgacgga tccggtaact caggaactga agacgatggc cggtttttac
gatcatctct ttggcttcaa gttgccgtgt aaactggcgg cgatgagtaa ccatccagga tcctcttcca aaatggtggt tctggcaaaa
ccaaagaagg gtgttgctag taacatcggc tttgaaccta ttcccgatcc tgctcatcct gtgttccggg tgagaagttc ctggccggag
ttgaagtacc tggaggggtt gttgtatctt cccgaagata caccactgac cattgaactg gcggaaacgt cggtcagttg tcagtctgtg
agttcagtcg ctttcgattt gaagaatctg acgactatct tgggtcgtgt tggtgaattc agggtgacgg cagatcaacc tttcaagctg
acgcccatta ttcctgagaa agaggaatcc ttcatcggga agacctacct cggtcttgat gctggagagc gatctggcgt tggtttcgcg
attgtgacgg ttgacggcga tgggtatgag gtgcagaggt tgggtgtgca tgaagatact cagcttatgg cgcttcagca agtcgccagc
aagtctctta aggagccggt tttccagcca ctccgtaagg gcacatttcg tcagcaggag cgcattcgca aaagcctccg cggttgctac
tggaatttct atcatgcatt gatgatcaag taccgagcta aagttgtgca tgaggaatcg gtgggttcat ccggtctggt ggggcagtgg
ctgcgtgcat ttcagaagga tctcaaaaag gctgatgttc tgcccaagaa gggtggaaaa aatggtgtag acaaaaaaaa gagagaaagc
agcgctcagg ataccttatg gggaggagct ttctcgaaga aggaagagca gcagatagcc tttgaggttc aggcagctgg atcaagccag
ttttgtctga agtgtggttg gtggtttcag ttggggatgc gggaagtaaa tcgtgtgcag gagagtggcg tggtgctgga ctggaaccgg
tccattgtaa ccttcctcat cgaatcctca ggagaaaagg tatatggttt cagtcctcag caactggaaa aaggctttcg tcctgacatc
gaaacgttca aaaaaatggt aagggatttt atgagacccc ccatgtttga tcgcaaaggt cggccggccg cggcgtatga aagattcgta
ctgggacgtc gtcaccgtcg ttatcgcttt gataaagttt ttgaagagag atttggtcgc agtgctcttt tcatctgccc gcgggtcggg
tgtgggaatt tcgatcactc cagtgagcag tcagccgttg tccttgccct tattggttac attgctgata aggaagggat gagtggtaag
aagcttgttt atgtgaggct ggctgaactt atggctgagt ggaagctgaa gaaactggag agatcaaggg tggaagaaca gagctcggca
caataa

Any of the gene editor effectors herein can also be tagged with Tev or any other suitable homing protein domains. According to Wolfs, et al. (Proc Natl Acad Sci USA. 2016 Dec. 27; 113(52):14988-14993. doi: 10.1073/pnas.1616343114. Epub 2016 Dec. 12), Tev is an RNA-guided dual active site nuclease that generates two noncompatible DNA breaks at a target site, effectively deleting the majority of the target site such that it cannot be regenerated.

The present invention provides for a composition for treating a lysogenic virus (budding virus) including a vector encoding two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors that target viral DNA, and RNA editors such as C2c2. Preferably, the composition includes isolated nucleic acid encoding a CRISPR-associated endonuclease (Cas9 or any other described above) and two or more gRNAs that are complementary to a target sequence in a lysogenic virus. Each gRNA can be complimentary to a different sequence within the lysogenic virus. The composition removes the replication critical segment of the viral genome (DNA) (or RNA using RNA editors such as C2c2) within the genome itself and translation products using RNA editors such as C2c2. Most preferably, the entire viral genome can be excised from the host cell infected with virus. Alternatively, additions, deletions, or mutations can be made in the genome of the virus. The composition can optionally include other CRISPR or gene editing systems that target DNA. The gRNAs are designed to be the most optimal in safety to provide no off target effects and no viral escape. The composition can treat any virus in the tables below that are indicated as having a lysogenic replication cycle, and is especially useful for retroviruses. The composition can be delivered by a vector or any other method as described below.

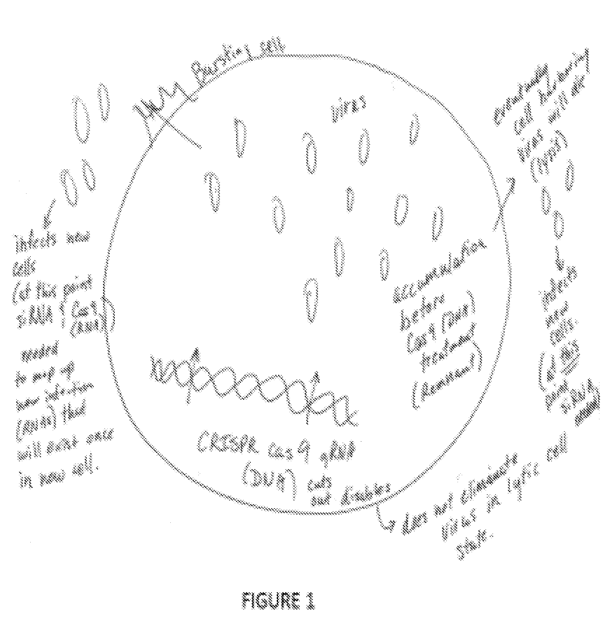

The present invention also provides for a composition for treating a lytic virus, including a vector encoding two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors for targeting viral DNA genomes for the excision of viral genes in virus that are lysogenic and either 1) small interfering RNA (siRNA)/microRNA (miRNA), short hairpin RNA, and interfering RNA (RNAi) (for RNA interference) that target critical RNAs (viral mRNA) that translate (non-coding or coding) viral proteins involved with the formation of viral proteins and/or virions or 2) CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors that target RNAs (viral mRNA), such as C2c2, that translate (non-coding or coding) viral proteins involved with the formation of virions. Preferably, the composition includes isolated nucleic acid encoding a CRISPR-associated endonuclease (Cas9), two or more gRNAs that are complementary to a target DNA sequence in a virus, and either the siRNA/miRNA/shRNAs/RNAi or CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors that is complementary to a target RNA sequence in the virus. Each gRNA can be complimentary to a different sequence within the virus. The composition can additionally include any other CRISPR or gene editing systems that target viral DNA genomes and excise segments of those genomes. This co-therapeutic is useful in treating individuals infected with lytic viruses that Cas9 systems alone cannot treat. As shown in FIG. 1, lytic and lysogenic viruses need to be treated in different ways. While CRISPR Cas9 is usually used to target DNA, this gene editing system can be designed to target RNA within the virus instead in order to target lytic viruses. For example, Nelles, et al. (Cell, Volume 165, Issue 2, p. 488-496, Apr. 7, 2016) shows that RNA-targeting Cas9 was able to bind mRNAs. Any of the lytic viruses listed in the tables below can be targeted with this composition. The composition can be delivered by a vector or any other method as described below.

The siRNA and C2c2 in the compositions herein is targeted to a particular gene in a virus or gene mRNA. The siRNA can have a first strand of a duplex substantially identical to the nucleotide sequence of a portion of the viral gene or gene mRNA sequence. The second strand of the siRNA duplex is complementary to both the first strand of the siRNA duplex and to the same portion of the viral gene mRNA. Isolated siRNA can include short double-stranded RNA from about 17 nucleotides to about 29 nucleotides in length, preferably from about 19 to about 25 nucleotides in length, that are targeted to the target mRNA. The siRNA's comprise a sense RNA strand and a complementary antisense RNA strand annealed together by standard Watson-Crick base-pairing interactions. The sense strand comprises a nucleic acid sequence which is substantially identical to a target sequence contained within the target mRNA. The siRNA of the invention can be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Alternatively, siRNA can also be expressed from recombinant circular or linear DNA plasmids using any suitable promoter. Suitable promoters for expressing siRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly. siRNA of the invention can be expressed from a recombinant plasmid either as two separate, complementary RNA molecules, or as a single RNA molecule with two complementary regions. For example, siRNA can be useful in targeting JC Virus, BKV, or SV40 polyomaviruses (U.S. Patent Application Publication No. 2007/0249552 to Khalili, et al.), wherein siRNA is used which targets JCV agnoprotein gene or large T antigen gene mRNA and wherein the sense RNA strand comprises a nucleotide sequence substantially identical to a target sequence of about 19 to about 25 contiguous nucleotides in agnoprotein gene or large T antigen gene mRNA.

The present invention also provides for a composition for treating both lysogenic and lytic viruses, including a vector encoding two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs, C2c2, C2c1, and other gene editors that target viral RNA. Preferably, the composition includes isolated nucleic acid encoding a CRISPR-associated endonuclease (Cas9) and two or more gRNAs that are complementary to a target RNA sequence in a virus. Each gRNA can be complimentary to a different sequence within the virus. The composition can additionally include any other CRISPR or gene editing systems that target viral RNA genomes and excise segments of those genomes. This composition can target viruses that have both lysogenic and lytic replication, as listed in the tables below.

The present invention provides for a composition for treating lytic viruses, including a vector encoding two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors and siRNA/miRNAs/shRNAs/RNAi (RNA interference) that target critical RNAs (viral mRNA) that translate (non-coding or coding) viral proteins involved with the formation of viral proteins and/or virions. Preferably, the composition includes isolated nucleic acid encoding a CRISPR-associated endonuclease (Cas9 or any other described above) and two or more gRNAs that are complementary to a target RNA sequence in a lytic virus. Each gRNA can be complimentary to a different sequence within the lytic virus. The composition can optionally include other CRISPR or gene editing systems that target viral RNA genomes and excise segments of those genomes for disruption in lytic viruses.

Various viruses can be targeted by the compositions and methods of the present invention. Depending on whether they are lytic or lysogenic, different compositions and methods can be used as appropriate.

TABLE 2 lists viruses in the picornaviridae/hepeviridae/flaviviridae families and their method of replication.

TABLE 2

| | | |
|---|---|---|
| Hepatitis A | +ssRNA viral genome | Lytic/Lysogenic Replication |
| | | cycle |
| Hepatitis B | dsDNA-RT viral genome | Lysogenic Replication |
| | | cycle |
| Hepatitis C | +ssRNA viral genome | Lytic Replication cycle |
| Hepatitis D | −ssRNA viral genome | Lytic/Lysogenic Replication |
| | | cycle |
| Hepatitis E | +ssRNA viral genome | |
| Coxsachievirus | | Lytic Replication cycle |

It should be noted that Hepatitis D propagates only in the presence of Hepatitis B, therefore, the composition particularly useful in treating Hepatitis D is one that targets Hepatitis B as well, such as two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors to treat the lysogenic virus and siRNAs/miRNAs/shRNAs/RNAi to treat the lytic virus.

TABLE 3 lists viruses in the herpesviridae family and their method of replication.

TABLE 3

| | | |
|---|---|---|
| HSV-1 (HHV1) | dsDNA viral genome | Lytic/Lysogenic Replication |
| | | cycle |
| HSV-2 (HHV2) | dsDNA viral genome | Lytic/Lysogenic Replication |
| | | cycle |
| Cytomegalovirus | dsDNA viral genome | Lytic/Lysogenic Replication |
| (HHV5) | | cycle |
| Epstein-Barr | dsDNA viral genome | Lytic/Lysogenic Replication |
| Virus (HHV4) | | cycle |
| Varicella Zoster | dsDNA viral genome | Lytic/Lysogenic Replication |
| Virus (HHV3) | | cycle |
| Roseolovirus | | |
| (HHV6A/B) | | |
| HHV7 | | |
| HHV8 | | |

TABLE 4 lists viruses in the orthomyxoviridae family and their method of replication.

TABLE 4

| | |
|---|---|
| Influenza Types A, B, C, D | −ssRNA viral genome |

TABLE 5 lists viruses in the retroviridae family and their method of replication.

TABLE 5

| | | |
|---|---|---|
| HIV1 and HIV2 | +ssRNA viral | Lytic/Lysogenic Replication |
| | genome | cycle |
| HTLV1 and HTLV2 | +ssRNA viral | Lytic/Lysogenic Replication |
| | genome | cycle |
| Rous Sarcoma | +ssRNA viral | Lytic/Lysogenic Replication |
| Virus | genome | cycle |

TABLE 6 lists viruses in the papillomaviridae family and their method of replication.

TABLE 6

| | | |
|---|---|---|
| HPV family | dsDNA viral genome | Budding from desquamating |
| | | cells (semi-lysogenic) |

TABLE 7 lists viruses in the flaviviridae family and their method of replication.

TABLE 7

| | | |
|---|---|---|
| Yellow Fever | +ssRNA viral genome | Budding/Lysogenic |
| | | Replication |
| Zika | +ssRNA viral genome | Budding/Lysogenic |
| | | Replication |
| Dengue | +ssRNA viral genome | Budding/Lysogenic |
| | | Replication |
| West Nile | +ssRNA viral genome | Budding/Lysogenic |
| | | Replication |
| Japanese Encephalitis | +ssRNA viral genome | Budding/Lysogenic |
| | | Replication |

TABLE 8 lists viruses in the reoviridae family and their method of replication.

TABLE 8

| | | |
|---|---|---|
| Rota | dsRNA viral genome | Lytic Replication cycle |
| Seadornvirus | dsRNA viral genome | Lytic Replication cycle |
| Coltivirus | dsRNA viral genome | Lytic Replication cycle |

TABLE 9 lists viruses in the rhabdoviridae family and their method of replication.

TABLE 9

| | | |
|---|---|---|
| Lyssa Virus (Rabies) | −ssRNA viral genome | Budding/Lysogenic |
| | | Replication |
| Vesiculovirus | −ssRNA viral genome | Budding/Lysogenic |
| | | Replication |
| Cytorhabdovirus | −ssRNA viral genome | Budding/Lysogenic |
| | | Replication |

TABLE 10 lists viruses in the bunyanviridae family and their method of replication.

TABLE 10

| | | |
|---|---|---|
| Hantaan Virus | tripartite −ssRNA viral | Budding/Lysogenic |
| | genome | Replication |
| Rift Valley Fever | tripartite −ssRNA viral | Budding/Lysogenic |
| | genome | Replication |
| Bunyamwera Virus | tripartite −ssRNA viral | Budding/Lysogenic |
| | genome | Replication |

TABLE 11 lists viruses in the arenaviridae family and their method of replication.

TABLE 11

| | | |
|---|---|---|
| Lassa Virus | ssRNA viral genome | Budding/Lysogenic |
| | | Replication |
| Junin Virus | ssRNA viral genome | Budding/Lysogenic |
| | | Replication |
| Machupo Virus | ssRNA viral genome | Budding/Lysogenic |
| | | Replication |
| Sabia Virus | ssRNA viral genome | Budding/Lysogenic |
| | | Replication |
| Tacaribe Virus | ssRNA viral genome | Budding/Lysogenic |
| | | Replication |
| Flexal Virus | ssRNA viral genome | Budding/Lysogenic |
| | | Replication |
| Whitewater Arroyo Virus | ssRNA viral genome | Budding/Lysogenic |
| | | Replication |

TABLE 12 lists viruses in the filoviridae family and their method of replication.

TABLE 12

| | | |
|---|---|---|
| Ebola | RNA viral genome | Budding/Lysogenic Replication |
| Marburg Virus | RNA viral genome | Budding/Lysogenic Replication |

TABLE 13 lists viruses in the polyomaviridae family and their method of replication.

TABLE 13

| | | |
|---|---|---|
| JC Virus | dsDNA circular viral | Lytic/Lysogenic Replication |
| | genome | cycle |
| BK Virus | dsDNA circular viral | Lytic/Lysogenic Replication |
| | genome | cycle |

The compositions of the present invention can be used to treat either active or latent viruses. The compositions of the present invention can be used to treat individuals in which latent virus is present but the individual has not yet presented symptoms of the virus. The compositions can target virus in any cells in the individual, such as, but not limited to, CD4+ lymphocytes, macrophages, fibroblasts, monocytes, T lymphocytes, B lymphocytes, natural killer cells, dendritic cells such as Langerhans cells and follicular dendritic cells, hematopoietic stem cells, endothelial cells, brain microglial cells, and gastrointestinal epithelial cells.

In the present invention, when any of the compositions are contained within a expression vector, the CRISPR endonuclease can be encoded by the same nucleic acid or vector as the gRNA sequences. Alternatively or in addition, the CRISPR endonuclease can be encoded in a physically separate nucleic acid from the gRNA sequences or in a separate vector.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Yeast expression systems can also be used. For example, the non-fusion pYES2 vector (XbaI, SphI, ShoI, NotI, GstXI, EcoRI, BstXI, BamHI, SacI, KpnI, and HindIII cloning sites; Invitrogen) or the fusion pYESHisA, B, C (XbaI, SphI, ShoI, NotI, BstXI, EcoRI, BamHI, SacI, KpnI, and HindIII cloning sites, N-terminal peptide purified with ProBond resin and cleaved with enterokinase; Invitrogen), to mention just two, can be employed according to the invention. A yeast two-hybrid expression system can also be prepared in accordance with the invention.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Suitable nucleic acid delivery systems include recombinant viral vector, typically sequence from at least one of an adenovirus, adenovirus-associated virus (AAV), helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, preferably about one polynucleotide. In some embodiments, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. The selection of appropriate promoters can readily be accomplished. An example of a suitable promoter is the 763-base-pair cytomegalovirus (CMV) promoter. Other suitable promoters which may be used for gene expression include, but are not limited to, the Rous sarcoma virus (RSV) (Davis, et al., Hum Gene Ther 4:151 (1993)), the SV40 early promoter region, the herpes thymidine kinase promoter, the regulatory sequences of the metallothionein (MMT) gene, prokaryotic expression vectors such as the β-lactamase promoter, the tac promoter, promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells, insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in myeloid cells, myelin basic protein gene control region which is active in oligodendrocyte cells in the brain, myosin light chain-2 gene control region which is active in skeletal muscle, and gonadotropic releasing hormone gene control region which is active in the hypothalamus. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

If desired, the polynucleotides of the invention can also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R.A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety.

As described above, the compositions of the present invention can be prepared in a variety of ways known to one of ordinary skill in the art. Regardless of their original source or the manner in which they are obtained, the compositions of the invention can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having a virus or at risk for contracting a virus. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, nucleic acids and vectors described herein in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of HIV infection, for example, brain macrophages, microglia, astrocytes, and gut-associated lymphoid cells. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the an isolated nucleic acid sequence comprising a sequence encoding a CRISPR-associated endonuclease and a guide RNA is operatively linked to a promoter or enhancer-promoter combination. Promoters and enhancers are described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

The present invention provides for a method of treating a lysogenic virus, by administering a composition including two or more CRISPR-associated nucleases such as Cas9, Cpf1, C2c1, and TevCas9 gRNAs, Argonaute endonuclease gDNAs and other gene editors that target viral DNA to an individual having a lysogenic virus, and inactivating the lysogenic virus. The lysogenic virus is integrated into the genome of the host cell and the composition inactivates the lysogenic virus by excising the viral DNA from the host cell. The composition can include any of the properties as described above, such as being in isolated nucleic acid, be packaged in a vector delivery system, or include other CRISPR or gene editing systems that target DNA. The lysogenic virus can be any listed in the tables above.

In any of the methods described herein, treatment can be in vivo (directly administering the composition) or ex vivo (for example, a cell or plurality of cells, or a tissue explant, can be removed from a subject having an viral infection and placed in culture, and then treated with the composition). Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems. In any of the methods described herein, the amount of the compositions administered is enough to inactivate all of the virus present in the individual. An individual is effectively treated whenever a clinically beneficial result ensues. This may mean, for example, a complete resolution of the symptoms of a disease, a decrease in the severity of the symptoms of the disease, or a slowing of the disease's progression. The present methods may also include a monitoring step to help optimize dosing and scheduling as well as predict outcome.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

The present invention also provides for a method for treating a lytic virus, including administering a vector encoding two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors that target viral DNA and a composition chosen from siRNAs/miRNAs/shRNAs/RNAi and CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors that target viral RNA to an individual having a lytic virus, and inactivating the lytic virus. The composition inactivates the lytic virus by excising the viral DNA and RNA from the host cell. The composition can include any of the properties as described above, such as being in isolated nucleic acid, be packaged in a vector delivery system, or include other CRISPR or gene editing systems that target DNA. The lytic virus can be any listed in the tables above.

The present invention also provides for a method for treating both lysogenic and lytic viruses, by administering a composition including a vector encoding two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors that target viral RNA to an individual having a lysogenic virus and lytic virus, and inactivating the lysogenic virus and lytic virus. The composition inactivates the viruses by excising the viral RNA from the host cell. The composition can include any of the properties as described above, such as being in isolated nucleic acid, or include other CRISPR or gene editing systems that target RNA. The lysogenic virus and lytic virus can be any listed in the tables above.

At the point of infection or when the virus has entered the cytoplasm, it can contain an RNA-based genome that is non-integrating (not converted to DNA), yet contributes to lysogenic type replication cycle. At this upstream point, the viral genome can be eliminated. On the other hand, the approach can be utilized to also target viral mRNA which occurs downstream (as the genome is translated). Although Argonaute is cited throughout the art, to this date it has not been modified to recognize RNA molecules.

The present invention provides for a method for treating lytic viruses, by administering a composition including a vector encoding two or more CRISPR-associated nucleases such as Cas9, Cpf1, C2c1, C2c3, TevCas9, Archaea Cas9, CasY.1-CasY.6, and CasX gRNAs, Argonaute endonuclease gDNAs and other gene editors that target viral RNA and siRNA/miRNAs/shRNAs/RNAi that target viral RNA to an individual having a lytic virus, and inactivating the lytic virus. The composition inactivates the lytic virus by excising the viral RNA from the host cell. The composition can include any of the properties as described above, such as being in isolated nucleic acid, or include other CRISPR or gene editing systems that target RNA. Two or more gene editors will be utilized that can target RNA to excise the RNA-based viral genome and/or the viral mRNA that occurs downstream. In the case of siRNA/miRNA/shRNA/RNAi which do not use a nuclease based mechanism, one or more are utilized for the degradative silencing on viral RNA transcripts (non-coding or coding) The lytic virus can be any listed in the tables above.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition for treating a lysogenic virus, comprising a vector encoding isolated nucleic acid encoding two or more gene editors chosen from the group consisting of gene editors that target viral DNA, gene editors that target viral RNA, and combinations thereof.

2. The composition of claim 1, wherein said gene editors that target viral DNA are chosen from the group consisting of CRISPR-associated nucleases and Argonaute endonuclease gDNAs.

3. The composition of claim 2, wherein said CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

4. The composition of claim 1, wherein said gene editors that target viral RNA are chosen from the group consisting of C2c2 and RNase P RNA.

5. The composition of claim 1, wherein said composition removes a replication critical segment of the viral DNA or RNA.

6. The composition of claim 1, wherein said composition excises an entire viral genome of said lysogenic virus from a host cell.

7. The composition of claim 1, wherein said lysogenic virus is chosen from the group consisting of hepatitis A, hepatitis B, hepatitis D, HSV-1, HSV-2, cytomegalovirus, Epstein-Barr virus, Varicella Zoster virus, HIV1, HIV2, HTLV1, HTLV2, Rous Sarcoma virus, HPV virus, yellow fever, zika, dengue, West Nile, Japanese encephalitis, lyssa virus, vesiculovirus, cytohabdovirus, Hantaan virus, Rift Valley virus, Bunyamwera virus, Lassa virus, Junin virus, Machupo virus, Sabia virus, Tacaribe virus, Flexal virus, Whitewater Arroyo virus, ebola, Marburg virus, JC virus, and BK virus.

8. A composition for treating a lytic virus, comprising a vector encoding isolated nucleic acid encoding at least one gene editor that targets viral DNA and a viral RNA targeting composition.

9. The composition of claim 8, wherein said gene editor that targets viral DNA is chosen from the group consisting of CRISPR-associated nucleases and Argonaute endonuclease gDNAs.

10. The composition of claim 9, wherein said CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

11. The composition of claim 8, wherein said viral RNA targeting composition is chosen from the group consisting of siRNAs, miRNAs, shRNAs, RNAi, CRISPR-associated nucleases, Argonaute endonuclease gDNAs, C2c2, and RNase P RNA.

12. The composition of claim 8, wherein said composition removes a replication critical segment of the viral DNA or RNA.

13. The composition of claim 8, wherein said composition excises an entire viral genome of said lytic virus from a host cell.

14. The composition of claim 8, wherein said lytic virus is chosen from the group consisting of hepatitis A, hepatitis C, hepatitis D, coxsachievirus, HSV-1, HSV-2, cytomegalovirus, Epstein-Barr virus, varicella zoster virus, HIV1, HIV2, HTLV1, HTLV2, Rous Sarcoma virus, rota, seadornvirus, coltivirus, JC virus, and BK virus.

15. A composition for treating both lysogenic and lytic viruses, comprising a vector encoding isolated nucleic acid encoding two or more gene editors that target viral RNA, chosen from the group consisting of CRISPR-associated nucleases, Argonaute endonuclease gDNAs, C2c2, RNase P RNA, and combinations thereof.

16. The composition of claim 15, wherein said CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

17. The composition of claim 15, wherein said composition removes a replication critical segment of the viral RNA.

18. The composition of claim 15, wherein said composition excises an entire viral genome of said lysogenic and lytic virus from a host cell.

19. The composition of claim 15, wherein said lysogenic and lytic virus is chosen from the group consisting of hepatitis A, hepatitis C, hepatitis D, HSV-1, HSV-2, cytomegalovirus, Epstein-Barr virus, varicella zoster virus, HIV1, HIV2, HTLV1, HTLV2, Rous Sarcoma virus, JC virus, and BK virus.

20. A composition for treating lytic viruses, comprising a vector encoding isolated nucleic acid encoding two or more gene editors that target viral RNA and a viral RNA targeting composition.

21. The composition of claim 20, wherein said gene editors that target viral RNA are chosen from the group consisting of CRISPR-associated nucleases and Argonaute endonuclease gDNAs.

22. The composition of claim 21, wherein said CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

23. The composition of claim 20, wherein said viral RNA targeting composition is chosen from the group consisting of siRNAs, miRNAs, shRNAs, RNAi, C2c2, and RNase P RNA.

24. The composition of claim 20, wherein said composition removes a replication critical segment of the viral RNA.

25. The composition of claim 20, wherein said composition excises an entire viral genome of said lytic virus from a host cell.

26. The composition of claim 20, wherein said lytic virus is chosen from the group consisting of hepatitis A, hepatitis C, hepatitis D, coxsachievirus, HSV-1, HSV-2, cytomegalovirus, Epstein-Barr virus, varicella zoster virus, HIV1, HIV2, HTLV1, HTLV2, Rous Sarcoma virus, rota, seadornvirus, coltivirus, JC virus, and BK virus.

27. A method of treating a lysogenic virus, including the steps of:
administering a composition including a vector encoding isolated nucleic acid encoding two or more gene editors chosen from the group consisting of gene editors that target viral DNA, gene editors that target viral RNA, and combinations thereof to an individual having a lysogenic virus; and
inactivating the lysogenic virus.

28. The method of claim 27, wherein the gene editors that target viral DNA are chosen from the group consisting of CRISPR-associated nucleases and Argonaute endonuclease gDNAs.

29. The method of claim 28, wherein the CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

30. The method of claim 27, wherein the gene editors that target viral RNA are chosen from the group consisting of C2c2 and RNase P RNA.

31. The method of claim 27, wherein said inactivating step includes removing a replication critical segment of the viral DNA or RNA.

32. The method of claim 27, wherein said inactivating step includes excising an entire viral genome of the lysogenic virus from a host cell.

33. The method of claim 27, wherein the lysogenic virus is chosen from the group consisting of hepatitis A, hepatitis B, hepatitis D, HSV-1, HSV-2, cytomegalovirus, Epstein-Barr virus, Varicella Zoster virus, HIV1, HIV2, HTLV1, HTLV2, Rous Sarcoma virus, HPV virus, yellow fever, zika, dengue, West Nile, Japanese encephalitis, lyssa virus, vesiculovirus, cytohabdovirus, Hantaan virus, Rift Valley virus, Bunyamwera virus, Lassa virus, Junin virus, Machupo virus, Sabia virus, Tacaribe virus, Flexal virus, Whitewater Arroyo virus, ebola, Marburg virus, JC virus, and BK virus.

34. A method for treating a lytic virus, including the steps of:
administering a composition including a vector encoding isolated nucleic acid encoding at least one gene editor that targets viral DNA and a viral RNA targeting composition to an individual having a lytic virus; and
inactivating the lytic virus.

35. The method of claim 34, wherein the gene editor that targets viral DNA is chosen from the group consisting of CRISPR-associated nucleases and Argonaute endonuclease gDNAs.

36. The method of claim 35, wherein the CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

37. The method of claim 34, wherein the viral RNA targeting composition is chosen from the group consisting of siRNAs, miRNAs, shRNAs, RNAi, CRISPR-associated nucleases, Argonaute endonuclease gDNAs, C2c2, and RNase P RNA.

38. The method of claim 34, wherein said inactivating step includes removing a replication critical segment of the viral DNA or RNA.

39. The method of claim 34, wherein said inactivating step includes excising an entire viral genome of the lytic virus from a host cell.

40. The method of claim 34, wherein the lytic virus is chosen from the group consisting of hepatitis A, hepatitis C, hepatitis D, coxsachievirus, HSV-1, HSV-2, cytomegalovirus, Epstein-Barr virus, varicella zoster virus, HIV1, HIV2, HTLV1, HTLV2, Rous Sarcoma virus, rota, seadornvirus, coltivirus, JC virus, and BK virus.

41. A method for treating both lysogenic and lytic viruses, including the steps of:
administering a composition including a vector encoding isolated nucleic acid encoding two or more gene editors that target viral RNA, chosen from the group consisting of CRISPR-associated nucleases, Argonaute endonuclease gDNAs, C2c2, RNase P RNA, and combinations thereof to an individual having a lysogenic virus and lytic virus; and
inactivating the lysogenic virus and lytic virus.

42. The method of claim 41, wherein said CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

43. The method of claim 41, wherein said inactivating step includes removing a replication critical segment of the viral RNA.

44. The method of claim 41, wherein said inactivating step includes excising an entire viral genome of the lysogenic and lytic virus from a host cell.

45. The method of claim 41, wherein the lysogenic and lytic virus is chosen from the group consisting of hepatitis A, hepatitis C, hepatitis D, HSV-1, HSV-2, cytomegalovirus, Epstein-Barr virus, varicella zoster virus, HIV1, HIV2, HTLV1, HTLV2, Rous Sarcoma virus, JC virus, and BK virus.

46. A method for treating lytic viruses, including the steps of:
administering a composition including a vector encoding isolated nucleic acid encoding two or more gene editors that target viral RNA and a viral RNA targeting composition to an individual having a lytic virus; and
inactivating the lytic virus.

47. The method of claim 46, wherein the gene editors that target viral RNA are chosen from the group consisting of CRISPR-associated nucleases and Argonaute endonuclease gDNAs.

48. The method of claim 47, wherein the CRISPR-associated nucleases are chosen from the group consisting of Cas9 gRNAs, Cpf1 gRNAs, C2c1 gRNAs, C2c3 gRNAs, TevCas9 gRNAs, Archaea Cas9 gRNAs, CasY.1 gRNAs, CasY.2 gRNAs, CasY.3 gRNAs, CasY.4 gRNAs, CasY.5 gRNAs, CasY.6 gRNAs, and CasX gRNAs.

49. The method of claim 46, wherein the viral RNA targeting composition is chosen from the group consisting of siRNAs, miRNAs, shRNAs, RNAi, C2c2, and RNase P RNA.

50. The method of claim 46, wherein said inactivating step includes removing a replication critical segment of the viral RNA.

51. The method of claim 46, wherein said inactivating step includes excising an entire viral genome of the lytic virus from a host cell.

52. The method of claim 46, wherein the lytic virus is chosen from the group consisting of hepatitis A, hepatitis C, hepatitis D, coxsachievirus, HSV-1, HSV-2, cytomegalovirus, Epstein-Barr virus, varicella zoster virus, HIV1, HIV2, HTLV1, HTLV2, Rous Sarcoma virus, rota, seadornvirus, coltivirus, JC virus, and BK virus.


SEQUENCE LISTING (20 sequences)

SEQ ID NO: 1 - protein, 949 aa, Artificial Sequence
  ARMAN 1
MRDSITAPRYSSALAARIKEFNSAFKLGIDLGTKTGGVALVKDNKVLLAKTFLDYHKQTLEERRIHRRNRRSRLARRKRIARLRSWILRQKIYGKQLPDPYKIKKMQLPNGVRKGENWIDLVVSGRDLSPEAFVRAITLIFQKRGQRYEEVAKEIEEMSYKEFSTHIKALTSVTEEEFTALAAEIERRQDVVDTDKEAERYTQLSELLSKVSESKSESKDRAQRKEDLGKVVNAFCSAHRIEDKDKWCKELMKLLDRPVRHARFLNKVLIRCNICDRATPKKSRPDVRELLYFDTVRNFLKAGRVEQNPDVISYYKKIYMDAEVIRVKILNKEKLTDEDKKQKRKLASELNRYKNKEYVTDAQKKMQEQLKTLLFMKLTGRSRYCMAHLKERAAGKDVEEGLHGVVQKRHDRNIAQRNHDLRVINLIESLLFDQNKSLSDAIRKNGLMYVTIEAPEPKTKHAKKGAAVVRDPRKLKEKLFDDQNGVCIYTGLQLDKLEISKYEKDHIFPDSRDGPSIRDNLVLTTKEINSDKGDRTPWEWMHDNPEKWKAFERRVAEFYKKGRINERKRELLLNKGTEYPGDNPTELARGGARVNNFITEFNDRLKTHGVQELQTIFERNKPIVQVVRGEETQRLRRQWNALNQNFIPLKDRAMSFNHAEDAAIAASMPPKFWREQIYRTAWHFGPSGNERPDFALAELAPQWNDFFMTKGGPIIAVLGKTKYSWKHSIIDDTIYKPFSKSAYYVGIYKKPNAITSNAIKVLRPKLLNGEHTMSKNAKYYHQKIGNERFLMKSQKGGSIITVKPHDGPEKVLQISPTYECAVLTKHDGKIIVKFKPIKPLRDMYARGVIKAMDKELETSLSSMSKHAKYKELHTHDIIYLPATKKHVDGYFIITKLSAKHGIKALPESMVKVKYTQIGSENNSEVKLTKPKPEITLDSEDITNIYNFTR

SEQ ID NO: 2 - DNA, 2851 bp, Artificial Sequence
  ARMAN 1
atgagagactctattactgcacctagatacagctccgctcttgccgccagaataaaggagtttaattctgctttcaagttaggaatcgacctaggaacaaaaaccggcggcgtagcactggtaaaagacaacaaagtgctgctcgctaagacattcctcgattaccataaacaaacactggaggaaaggaggatccatagaagaaacagaaggagcaggctagccaggcggaagaggattgctcggctgcgatcatggatactcagacagaagatttatggcaagcagcttcctgacccatacaaaatcaaaaaaatgcagttgcctaatggtgtacgaaaaggggaaaactggattgacctggtagtttctggacgggacctttcaccagaagccttcgtgcgtgcaataactctgatattccaaaagagagggcaaagatatgaagaagtggccaaagagatagaagaaatgagttacaaggaatttagtactcacataaaagccctgacatccgttactgaagaagaatttactgctctggcagcagagatagaacggaggcaggatgtggttgacacagacaaggaggccgaacgctatacccaattgtctgagttgctctccaaggtctcagaaagcaaatctgaatctaaagacagagcgcagcgtaaggaggatctcggaaaggtggtgaacgctttctgcagtgctcatcgtatcgaagacaaggataaatggtgtaaagaacttatgaaattactagacagaccagtcagacacgctaggttccttaacaaagtactgatacgttgcaatatctgcgatagggcaacccctaagaaatccagacctgacgtgagggaactgctatattttgacacagtaagaaacttcttgaaggctggaagagtggagcaaaacccagacgttattagttactataaaaaaatttatatggatgcagaagtaatcagggtcaaaattctgaataaggaaaagctgactgatgaggacaaaaagcaaaagaggaaattagcgagcgaacttaacaggtacaaaaacaaagaatacgtgactgatgcgcagaagaagatgcaagagcaacttaagacattgctgttcatgaagctgacaggcaggtctagatactgcatggctcatcttaaggaaagggcagcaggcaaagatgtagaagaaggacttcatggcgttgtgcagaaaagacacgacaggaacatagcacagcgcaatcacgacttacgtgtgattaatcttattgagagtctgcttttcgaccaaaacaaatcgctctccgatgcaataaggaagaacgggttaatgtatgttactattgaggctccagagccaaagactaagcacgcaaagaaaggcgcagctgtggtaagggatcccagaaagttgaaggagaagttgtttgatgatcaaaacggcgtttgcatatatacgggcttgcagttagacaaattagagataagtaaatacgagaaggaccatatctttccagattcaagggatggaccatctatcagggacaatcttgtactcactacaaaagagataaattcagacaaaggcgataggaccccatgggaatggatgcatgataacccagaaaaatggaaagcgttcgagagaagagtcgcagaattctataagaaaggcagaataaatgagaggaaaagagaactcctattaaacaaaggcactgaataccctggcgataacccgactgagctggcgcggggaggcgcccgtgttaacaactttattactgaatttaatgaccgcctcaaaacgcatggagtccaggaactgcagaccatctttgagcgtaacaaaccaatagtgcaggtagtcaggggtgaagaaacgcagcgtctgcgcagacaatggaatgcactaaaccagaatttcataccactaaaggacagggcaatgtcgttcaaccacgctgaagacgcagccatagcagcaagcatgccaccaaaattctggagggagcagatataccgtactgcgtggcactttggacctagtggaaatgagagaccggactttgctttggcagaattggcgccacaatggaatgacttctttatgactaagggcggtccaataatagcagtgctgggcaaaacgaagtatagttggaagcacagcataattgatgacactatatacaagccattcagcaaaagtgcttactatgttgggatatacaaaaagccgaacgccatcacgtccaatgctataaaagtcttaaggccaaaactcttaaatggcgaacatacaatgtctaagaatgcaaagtattatcatcagaagattggtaatgagcgcttcctcatgaaatctcagaaaggtggatcgataattacagtaaaaccacacgacggaccggaaaaagtgcttcaaatcagccctacatatgaatgcgcagtccttactaagcatgacggtaaaataatagtcaaatttaaaccaataaagccgctacgggacatgtatgcccgcggtgtgattaaagccatggacaaagagcttgaaacaagcctctctagcatgagtaaacacgctaagtacaaggagttacacactcatgatatcatatatctgcctgctacaaagaagcacgtagatggctacttcataataaccaaactaagtgcgaaacatggcataaaagcactccccgaaagcatggttaaagtcaagtatactcaaattgggagtgaaaacaatagtgaagtgaagcttaccaaaccaaaaccagagataactttggatagtgaagatattacaaacatatataatttcacccgctaag

SEQ ID NO: 3 - protein, 967 aa, Artificial Sequence
  ARMAN 4
MLGSSRYLRYNLTSFEGKEPFLIMGYYKEYNKELSSKAQKEFNDQISEFNSYYKLGIDLGDKTGIAIVKGNKIILAKTLIDLHSQKLDKRREARRNRRTRLSRKKRLARLRSWVMRQKVGNQRLPDPYKIMHDNKYWSIYNKSNSANKKNWIDLLIHSNSLSADDFVRGLTIIFRKRGYLAFKYLSRLSDKEFEKYIDNLKPPISKYEYDEDLEELSSRVENGEIEEKKFEGLKNKLDKIDKESKDFQVKQREEVKKELEDLVDLFAKSVDNKIDKARWKRELNNLLDKKVRKIRFDNRFILKCKIKGCNKNTPKKEKVRDFELKMVLNNARSDYQISDEDLNSFRNEVINIFQKKENLKKGELKGVTIEDLRKQLNKTFNKAKIKKGIREQIRSIVFEKISGRSKFCKEHLKEFSEKPAPSDRINYGVNSAREQHDFRVLNFIDKKIFKDKLIDPSKLRYITIESPEPETEKLEKGQISEKSFETLKEKLAKETGGIDIYTGEKLKKDFEIEHIFPRARMGPSIRENEVASNLETNKEKADRTPWEWFGQDEKRWSEFEKRVNSLYSKKKISERKREILLNKSNEYPGLNPTELSRIPSTLSDFVESIRKMFVKYGYEEPQTLVQKGKPIIQVVRGRDTQALRWRWHALDSNIIPEKDRKSSFNHAEDAVIAACMPPYYLRQKIFREEAKIKRKVSNKEKEVTRPDMPTKKIAPNWSEFMKTRNEPVIEVIGKVKPSWKNSIMDQTFYKYLLKPFKDNLIKIPNVKNTYKWIGVNGQTDSLSLPSKVLSISNKKVDSSTVLLVHDKKGGKRNWVPKSIGGLLVYITPKDGPKRIVQVKPATQGLLIYRNEDGRVDAVREFINPVIEMYNNGKLAFVEKENEEELLKYFNLLEKGQKFERIRRYDMITYNSKFYYVTKINKNHRVTIQEESKIKAESDKVKSSSGKEYTRKETEELSLQKLAELISI

SEQ ID NO: 4 - DNA, 2906 bp, Artificial Sequence
  ARMAN 4
atgttaggctccagcaggtacctccgttataacctaacctcgtttgaaggcaaggagccatttttaataatgggatattacaaagagtataataaggaattaagttccaaagctcaaaaagaatttaatgatcaaatttctgaatttaattcgtattacaaactaggtatagatctcggagataaaacaggaattgcaatcgtaaagggcaacaaaataatcctagcaaaaacactaattgatttgcattcccaaaaattagataaaagaagggaagctagaagaaatagaagaactcggctttccagaaagaaaaggcttgcgagattaagatcgtgggtaatgcgtcagaaagttggcaatcaaagacttcccgatccatataaaataatgcatgacaataagtactggtctatatataataagagtaattctgcaaataaaaagaattggatagatctgttaatccacagtaactctttatcagcagacgattttgttagaggcttaactataattttcagaaaaagaggctatttagcatttaagtatctttcaaggttaagcgataaggaatttgaaaaatacatagataacttaaaaccacctataagcaaatacgagtatgatgaggatttagaagaattatcaagcagggttgaaaatggggaaatagaggaaaagaaattcgaaggcttaaagaataagctagataaaatagacaaagaatctaaagactttcaagtaaagcaaagagaagaagtaaaaaaggaactggaagacttagttgatttgtttgctaaatcagttgataataaaatagataaagctaggtggaaaagggagctaaataatttattggataagaaagtaaggaaaatacggtttgacaaccgctttattttgaagtgcaaaattaagggctgtaacaagaatactccaaagaaagagaaggtcagagattttgaattgaagatggttttaaataatgctagaagcgattatcagatttctgatgaggatttaaactcttttagaaatgaagtaataaatatatttcaaaagaaggaaaacttaaagaaaggagagctgaaaggagttactattgaagatttgagaaagcagcttaataaaacttttaataaagccaagattaaaaaagggataagggagcagataaggtctatcgtgtttgaaaaaattagtggaaggagtaaattctgcaaagaacatctaaaagaattttctgagaagccggctccttctgacaggattaattatggggttaattcagcaagagaacaacatgattttagagtcttaaatttcatagataaaaaaatattcaaagataagttgatagatccctcaaaattgaggtatataactattgaatctccagaaccagaaacagagaagttggaaaaaggtcaaatatcagagaagagcttcgaaacattgaaagaaaaattggctaaagaaacaggtggtattgatatatacactggtgaaaaattaaagaaagactttgaaatagagcacatattcccaagagcaaggatggggccttctataagggaaaacgaagtagcatcaaatctggaaacaaataaggaaaaggccgatagaactccttgggaatggtttgggcaagatgaaaaaagatggtcagagtttgagaaaagagttaattctctttatagtaaaaagaaaatatcagagagaaaaagagaaattttgttaaataagagtaatgaatatccgggattaaaccctacagaactaagtagaatacctagtacgctgagcgacttcgttgagagtataagaaaaatgtttgttaagtatggctatgaagagcctcaaactttggttcaaaaaggaaaaccgataatacaagttgttagaggcagagacacacaagctttgaggtggagatggcatgcattagatagtaatataataccagaaaaggacaggaaaagttcatttaatcacgctgaagatgcagttattgccgcctgtatgccaccttactatctcaggcaaaaaatatttagagaagaagcaaaaataaaaagaaaagtaagcaataaggaaaaggaagttacacggcctgacatgcctactaaaaagatagctccgaactggtcggaatttatgaaaactagaaatgagccggttattgaagtaataggaaaagttaagccaagctggaaaaacagcataatggatcaaacattttataaatatcttttgaagccatttaaagataacctgataaaaatacccaacgttaaaaatacatacaagtggataggagttaatggacaaactgattcattatccctcccgagtaaggtcttatctatctctaataaaaaggttgattcttctacagttcttcttgtgcatgataagaagggtggtaagcggaattgggtacctaaaagtatagggggtttgttggtatatataactcctaaagacgggccgaaaagaatagttcaagtaaagccagcaactcagggtttgttaatatatagaaatgaagatggcagagtagatgctgtaagagagttcataaatccagtgatagaaatgtataataatggcaaattggcatttgtagaaaaagaaaatgaagaagagcttttgaaatattttaatttgctggaaaaaggtcaaaaatttgaaagaataagacggtatgatatgataacctacaatagtaaattttactatgtaacaaaaataaacaagaatcacagagttactatacaagaagagtctaagataaaagcagaatcagacaaagttaagtcctcttcaggcaaagagtatactcgtaaggaaaccgaggaattatcacttcaaaaattagcggaattaattagtatataaaa

SEQ ID NO: 5 - protein, 978 aa, Artificial Sequence
  CasX.1
MQEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

SEQ ID NO: 6 - DNA, 5495 bp, Artificial Sequence
  CasX.1
atgcttcttatttatcggagatatcttcaaacaccatcaacatggcaatggtgaaccattaatattctttgatgcttcttatttatcggagatatcttcaaacattgcccattttacaggcatatcttctggctctttgatgcttcttatttatcggagatatcttcaaacgtaatgtattgagaaagacatcaagattagataactttgatgcttcttatttatcggagatatcttcaaacacagaaacctgcaaagattgtatatatataagctttgatgcttcttatttatcggagatatcttcaaacgatacgtattttagcccgtctatttggggattaactttgatgcttcttatttatcggagatatcttcaaaccccgcatatccagatttttcaatgacttctggaaattgtattttcaatattttacaagttgcggaggatacctttaataatttagcagagttacgcactgtaaacctgttcttctcacaaaaagctttaacatcagattttcaaagaacttcttatgtaatttataagaatctaaaaaaacagctctgggtttgcatccagaactctccgataaataagcgctttacccatacgacatagtcgctggtgatggctctcaaagtaatgagataaaagcgccagtaataatttactattcacaaatcctttcgtcaagcttaaaatcaatcaaagaccatatccccttcattccaaatagcagcgcttccgtacctttctatccgttcatatatctcctctgagagaggataaattaccagacttatagagccatccataaatcctttttctttaaggttgagctttagatcagcccaccttgcttttgaaaggttaaactcaaagacagaatattgaatccgaacaccataggcttccagaagtttaactaaccgtgccctgaccttatcatcttcaatatcataacaaatgagatgtcgcattttaaagctctataggcttataacattccctatcatcttgaatatgctggctaaacaacctaacctgccgctcaactgcgtgctgatacgttattgattggataagtaaattggttttctgctcatctaccttaaagaattgatgccattttttgattacttttggataggcatccttattcagccaaacacctttttggtcagtttctttcctgaaatcgtctgtatccacttcccttctatttatcaaattgatcacaaaacggtcagccaacggccgccactcctccagaagatcgcatattaaagagggacgaccataatagacgtcatgcaagtaaccaaaggccgggtcaaaaccgacgagtaatgcagtcgaatgtatttcgttgaacaggagggtgtagataaggctcatcatggcgttgatttcatcctcaggaggtctcttggtacggcgcacaaaaacaaagcttggatgctttaagatagccgaaaaattgccataatactgccttgttgttgcgccttctattccacgcaaggtctctaaatcagtgacggcgttgatttcggtacactcgattctcaaaccaagtctatatttatcaagtaatgattgctggtttttgatcttaccggcaacgatactttttgcaatttcaagttttttgtggggatcaaaatgcttatgaatttgcgcccgacgaataaacagatttttgacgggttcaaattgaaggctcccttgatattcccatctgccgctaaagaaatgtatcggtatagattattctctgcaaaggctaataacacggctatcgagggtaacccggccaactaccacgatatcttttaccttcattgcgggaatcttctgccccttctcttcattgtccttttttatgagaaatgcccgaccacgacaatccaaaatgaattcatcacccgtgagatagagggttatcctgtcggttatagcggtcatcagtaagccttttatttttctaaccaagtattgaaggaagacacgattcactatactggcactgcggacacctatggtcatcaaccttgggaaacctgcttatatcaaaggacaagaagcagtctcgcagatttgtaacaacttctacacaacgcactttcagggttttatctataacaatttctttccgtctccgtgtttcacagaaaaatatttcaccaactggtatattgacattatacatctcttcaaggcaaattgcctgtaacccaatctgaacgtggaagttctcaaaatcccttaccttccctgtctttgtttcgataggaatcggtatcccatccctccactcgataaggtctgcccggcctgccaaaccgagcttattgctgtaaagatacacgcctgttacctgcttacaatcagggcagcttctctgcgatgatttatccaccgccctgtgcgcgtgtatggcctctgtaaagtggatgctcttagccatattacgccgttctccaacaaaggcataccatgcattgcgcggacaatagattgactccattaccgtgctgatgtgcaatatcagacggctggtttccatacttctttgagcttctttctgtaaaaggattgccatgtttcaacaaatgcccttttgtcagtatttccggtcgttttattggtttgatacttcttatattcttgagaacggagaaagagccacgaccttgcaatattcagtgctgcttgttcgtctgcatgggtttcaaaaccacagttcaggcaaacaaacttttcctgcaccggcctgtgactaaatctcttttttagcagagataaagcttcaccactgcggccttttgtccaactagaaatatcattatttaccgactcttccgaaagtctatccagctctacagagaggtcttttaccacattctgccttttataccggttatagtatgttatctgtccttcaacttttaactcttttccattgattgtagtcatccatccagtagccgtcttcttgagcttttcgagcaccctgtcataatctgcacttgtgattgtaaaaccacaattagaacatgtctttgaggtatactgtgccagagtctttgaaagataggtttttgatggcagaccttcataggcaagctttgcagtcagccagtcttccatcctcgtgtactgcctttccgccataaaagtcctcttgccttgtctaccaaaaccgcgggaaagattttcaaaaatgagcattgcatcttgagtaacagcataatataagaggtcacgagctgtatttcttaccatatcgtccgccagattcttcgcctttgatgcatattttctcgaatatccgcctgcccgcctttgttcaacttctttagcagcctgaatagtccgttgtttttccttataactttctcctattcgcaaaatatgcgttggattgcccaatgaatctttgaatcttgacaaggggcatccttccgggtctgttaatgctatgactgccgggatattttctccccggtctattcctatcagattcatcggttttatattcgatgagtcaagcacctctcttctttcaaatgtcagggcaacaaaaagtgctggttcatcctgtctcgtccttctgttatagagcgttttttcaataaccctgccattggcgagtttcaatgaacccgtctcaaggctcaataggtcgttccagataaactccctcccctgcctttttccaaaggccaaaggcagaattatcaaattcgggtcatcaaaattgaagttgacctccataggcacaatctcaccgctttttttattaattactgtataaaacctatttgcttcaaaagcttctggcttgatttttttgaagcgtagcttaccacctttgaagtaatttattattaaataaagatttaacttctttacgccgtctttctgccatataaatgcacaattatactgtttagaaaatccgcttatatctaaaatgctgttctctgcttctatagcaaatggttttcctctcaaatctccataccacttttgaagctttaactcacacctgcaaaactcatccttatcagcttctttgagcccttcaataacaaaagaggcctttgccctgagccaatcagtgagggcagcctttgattgagcatcttcagaccttctttcttcctccaactttatgtgcttactcagaccttcaacttttttatctattctttcccatgcctcatcataaactttgccccaatcttcaccgtgtttcttttcaaggtgaagcaaaaggtcaccaaactgataacgcgcaaacttttttccttttttacggtcttcttcagacgaaagatatggaagcaaggcttcctgccttttatatccagcaagattttgccagaagaccttcccgtcctctttcttttcgttaatcaactttttgacattacagaccatatcccaccaatcaacctcattcgcctggcgttcaacaagagggaaggacggaaaacccttaagccgctgtaagggctttgcctcatccctgccaattttgagtttctgccaaagattcaggtttacccagatcactatctgagcaacaacattgttataagcttcaatcccttcttttgtatgcggttgcggtggaagagtgattttaggaaatgcaagcccgtttgcacttgctatatcctttagatttgccaatctcttttcgtttttttttataaccttttggtgttcgaggatgatgtcctggtactttgtaaggaaactggctactgctcccatacaggcatcagataaagccttaccaacgggaccacttgcgcagctattgccaccgatctgttctagcggctttacaggatggttcgattctcttgttacgtggattgaataaaagtccaatgccctttgaccgaacttccccaacgaatacgttactagctcgtcatttgcctccggtttatgcggcgagagcaatatcaaacgttcatgctcggagacattacaacggccaaagtaatttgtatggggcttacccttgtcattcacttgttcaagcttataaacatagaggggttgacagcactgagaacaggcaaatccagaacttgttagtctctcatttccgtccttcaccggaatcaattttctctgatcaatattcttgggcgctggttgtgcaaccctgctcatcaatccgacagggtctttttggaactcttcccaataaacatgcaggattgctttcttcatttccgtatagtcagtgaggagtttatttaaatttgcacgtgaagtatttgaaatgggctgaggaatgttttccggctttttgcgaagattctctaacctttctctcaggtcaggtgtcataacccgaacgagcaaggttttcatagggccggttttgccggcttttttcgtgttgctatcctttaccaatctccttcgtattttatttatcctttttatttcctgcatcttt

SEQ ID NO: 7 - protein, 986 aa, Artificial Sequence
  CasX.1 deltaproteobacteria
MEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASKKIDQNKLKPEMDEKGNLTTAGFACSQCGQPLFVYKLEQVSEKGKAYTNYFGRCNVAEHEKLILLAQLKPEKDSDEAVTYSLGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVDRGENIPAVIALTDPEGCPLPEFKDSSGGPTDILRIGEGYKEKQRAIQAAKEVEQRRAGGYSRKFASKSRNLADDMVRNSARDLFYHAVTHDAVLVFENLSRGFGRQGKRTFMTERQYTKMEDWLTAKLAYEGLTSKTYLSKTLAQYTSKTCSNCGFTITTADYDGMLVRLKKTSDGWATTLNNKELKAEGQITYYNRYKRQTVEKELSAELDRLSEESGNNDISKWTKGRRDEALFLLKKRFSHRPVQEQFVCLDCGHEVHADEQAALNIARSWLFLNSNSTEFKSYKSGKQPFVGAWQAFYKRRLKEVWKPNA

SEQ ID NO: 8 - DNA, 2962 bp, Artificial Sequence
  CasX.1 deltaproteobacteria
atggaaaagagaataaacaagatacgaaagaaactatcggccgataatgccacaaagcctgtgagcaggagcggccccatgaaaacactccttgtccgggtcatgacggacgacttgaaaaaaagactggagaagcgtcggaaaaagccggaagttatgccgcaggttatttcaaataacgcagcaaacaatcttagaatgctccttgatgactatacaaagatgaaggaggcgatactacaagtttactggcaggaatttaaggacgaccatgtgggcttgatgtgcaaatttgcccagcctgcttccaaaaaaattgaccagaacaaactaaaaccggaaatggatgaaaaaggaaatctaacaactgccggttttgcatgttctcaatgcggtcagccgctatttgtttataagcttgaacaggtgagtgaaaaaggcaaggcttatacaaattacttcggccggtgtaatgtggccgagcatgagaaattgattcttcttgctcaattaaaacctgaaaaagacagtgacgaagcagtgacatactcccttggcaaattcggccagagggcattggacttttattcaatccacgtaacaaaagaatccacccatccagtaaagcccctggcacagattgcgggcaaccgctatgcaagcggacctgttggcaaggccctttccgatgcctgtatgggcactatagccagttttctttcgaaatatcaagacatcatcatagaacatcaaaaggttgtgaagggtaatcaaaagaggttagagagtctcagggaattggcagggaaagaaaatcttgagtacccatcggttacactgccgccgcagccgcatacgaaagaaggggttgacgcttataacgaagttattgcaagggtacgtatgtgggttaatcttaatctgtggcaaaagctgaagctcagccgtgatgacgcaaaaccgctactgcggctaaaaggattcccatctttccctgttgtggagcggcgtgaaaacgaagttgactggtggaatacgattaatgaagtaaaaaaactgattgacgctaaacgagatatgggacgggtattctggagcggcgttaccgcagaaaagagaaataccatccttgaaggatacaactatctgccaaatgagaatgaccataaaaagagagagggcagtttggaaaaccctaagaagcctgccaaacgccagtttggagacctcttgctgtatcttgaaaagaaatatgccggagactggggaaaggtcttcgatgaggcatgggagaggatagataagaaaatagccggactcacaagccatatagagcgcgaagaagcaagaaacgcggaagacgctcaatccaaagccgtacttacagactggctaagggcaaaggcatcatttgttcttgaaagactgaaggaaatggatgaaaaggaattctatgcgtgtgaaatccaacttcaaaaatggtatggcgatcttcgaggcaacccgtttgccgttgaagctgagaatagagttgttgatataagcgggttttctatcggaagcgatggccattcaatccaatacagaaatctccttgcctggaaatatctggagaacggcaagcgtgaattctatctgttaatgaattatggcaagaaagggcgcatcagatttacagatggaacagatattaaaaagagcggcaaatggcagggactattatatggcggtggcaaggcaaaggttattgatctgactttcgaccccgatgatgaacagttgataatcctgccgctggcctttggcacaaggcaaggccgcgagtttatctggaacgatttgctgagtcttgaaacaggcctgataaagctcgcaaacggaagagttatcgaaaaaacaatctataacaaaaaaatagggcgggatgaaccggctctattcgttgccttaacatttgagcgccgggaagttgttgatccatcaaatataaagcctgtaaaccttataggcgttgaccgcggcgaaaacatcccggcggttattgcattgacagaccctgaaggttgtcctttaccggaattcaaggattcatcagggggcccaacagacatcctgcgaataggagaaggatataaggaaaagcagagggctattcaggcagcaaaggaggtagagcaaaggcgggctggcggttattcacggaagtttgcatccaagtcgaggaacctggcggacgacatggtgagaaattcagcgcgagaccttttttaccatgccgttacccacgatgccgtccttgtctttgaaaacctgagcaggggttttggaaggcagggcaaaaggaccttcatgacggaaagacaatatacaaagatggaagactggctgacagcgaagctcgcatacgaaggtcttacgtcaaaaacctacctttcaaagacgctggcgcaatatacgtcaaaaacatgctccaactgcgggtttactataacgactgccgattatgacgggatgttggtaaggcttaaaaagacttctgatggatgggcaactaccctcaacaacaaagaattaaaagccgaaggccagataacgtattataaccggtataaaaggcaaaccgtggaaaaagaactctccgcagagcttgacaggctttcagaagagtcgggcaataatgatatttctaagtggaccaagggtcgccgggacgaggcattatttttgttaaagaaaagattcagccatcggcctgttcaggaacagtttgtttgcctcgattgcggccatgaagtccacgccgatgaacaggcagccttgaatattgcaaggtcatggctttttctaaactcaaattcaacagaattcaaaagttataaatcgggtaaacagcccttcgttggtgcttggcaggccttttacaaaaggaggcttaaagaggtatggaagcccaacgcctgat

SEQ ID NO: 9 - protein, 1125 aa, Artificial Sequence
  CasY.1
MRKKLFKGYILHNKRLVYTGKAAIRSIKYPLVAPNKTALNNLSEKIIYDYEHLFGPLNVASYARNSNRYSLVDFWIDSLRAGVIWQSKSTSLIDLISKLEGSKSPSEKIFEQIDFELKNKLDKEQFKDIILLNTGIRSSSNVRSLRGRFLKCFKEEFRDTEEVIACVDKWSKDLIVEGKSILVSKQFLYWEEEFGIKIFPHFKDNHDLPKLTFFVEPSLEFSPHLPLANCLERLKKFDISRESLLGLDNNFSAFSNYFNELFNLLSRGEIKKIVTAVLAVSKSWENEPELEKRLHFLSEKAKLLGYPKLTSSWADYRMIIGGKIKSWHSNYTEQLIKVREDLKKHQIALDKLQEDLKKVVDSSLREQIEAQREALLPLLDTMLKEKDFSDDLELYRFILSDFKSLLNGSYQRYIQTEEERKEDRDVTKKYKDLYSNLRNIPRFFGESKKEQFNKFINKSLPTIDVGLKILEDIRNALETVSVRKPPSITEEYVTKQLEKLSRKYKINAFNSNRFKQITEQVLRKYNNGELPKISEVFYRYPRESHVAIRILPVKISNPRKDISYLLDKYQISPDWKNSNPGEVVDLIEIYKLTLGWLLSCNKDFSMDFSSYDLKLFPEAASLIKNFGSCLSGYYLSKMIFNCITSEIKGMITLYTRDKFVVRYVTQMIGSNQKFPLLCLVGEKQTKNFSRNWGVLIEEKGDLGEEKNQEKCLIFKDKTDFAKAKEVEIFKNNIWRIRTSKYQIQFLNRLFKKTKEWDLMNLVLSEPSLVLEEEWGVSWDKDKLLPLLKKEKSCEERLYYSLPLNLVPATDYKEQSAEIEQRNTYLGLDVGEFGVAYAVVRIVRDRIELLSWGFLKDPALRKIRERVQDMKKKQVMAVFSSSSTAVARVREMAIHSLRNQIHSIALAYKAKIIYEISISNFETGGNRMAKIYRSIKVSDVYRESGADTLVSEMIWGKKNKQMGNHISSYATSYTCCNCARTPFELVIDNDKEYEKGGDEFIFNVGDEKKVRGFLQKSLLGKTIKGKEVLKSIKEYARPPIREVLLEGEDVEQLLKRRGNSYIYRCPFCGYKTDADIQAALNIACRGYISDNAKDAVKEGERKLDYILEVRKLWEKNGAVLRSAKFL

SEQ ID NO: 10 - DNA, 3380 bp, Artificial Sequence
  CasY.1
atgcgcaaaaaattgtttaagggttacattttacataataagaggcttgtatatacaggtaaagctgcaatacgttctattaaatatccattagtcgctccaaataaaacagccttaaacaatttatcagaaaagataatttatgattatgagcatttattcggacctttaaatgtggctagctatgcaagaaattcaaacaggtacagccttgtggatttttggatagatagcttgcgagcaggtgtaatttggcaaagcaaaagtacttcgctaattgatttgataagtaagctagaaggatctaaatccccatcagaaaagatatttgaacaaatagattttgagctaaaaaataagttggataaagagcaattcaaagatattattcttcttaatacaggaattcgttctagcagtaatgttcgcagtttgagggggcgctttctaaagtgttttaaagaggaatttagagataccgaagaggttatcgcctgtgtagataaatggagcaaggaccttatcgtagagggtaaaagtatactagtgagtaaacagtttctttattgggaagaagagtttggtattaaaatttttcctcattttaaagataatcacgatttaccaaaactaactttttttgtggagccttccttggaatttagtccgcacctccctttagccaactgtcttgagcgtttgaaaaaattcgatatttcgcgtgaaagtttgctcgggttagacaataatttttcggccttttctaattatttcaatgagctttttaacttattgtccaggggggagattaaaaagattgtaacagctgtccttgctgtttctaaatcgtgggagaatgagccagaattggaaaagcgcttacattttttgagtgagaaggcaaagttattagggtaccctaagcttacttcttcgtgggcggattatagaatgattattggcggaaaaattaaatcttggcattctaactataccgaacaattaataaaagttagagaggacttaaagaaacatcaaatcgcccttgataaattacaggaagatttaaaaaaagtagtagatagctctttaagagaacaaatagaagctcaacgagaagctttgcttcctttgcttgataccatgttaaaagaaaaagatttttccgatgatttagagctttacagatttatcttgtcagattttaagagtttgttaaatgggtcttatcaaagatatattcaaacagaagaggagagaaaggaggacagagatgttaccaaaaaatataaagatttatatagtaatttgcgcaacatacctagattttttggggaaagtaaaaaggaacaattcaataaatttataaataaatctctcccgaccatagatgttggtttaaaaatacttgaggatattcgtaatgctctagaaactgtaagtgttcgcaaacccccttcaataacagaagagtatgtaacaaagcaacttgagaagttaagtagaaagtacaaaattaacgcctttaattcaaacagatttaaacaaataactgaacaggtgctcagaaaatataataacggagaactaccaaagatctcggaggttttttatagatacccgagagaatctcatgtggctataagaatattacctgttaaaataagcaatccaagaaaggatatatcttatcttctcgacaaatatcaaattagccccgactggaaaaacagtaacccaggagaagttgtagatttgatagagatatataaattgacattgggttggctcttgagttgtaacaaggatttttcgatggatttttcatcgtatgacttgaaactcttcccagaagccgcttccctcataaaaaattttggctcttgcttgagtggttactatttaagcaaaatgatatttaattgcataaccagtgaaataaaggggatgattactttatatactagagacaagtttgttgttagatatgttacacaaatgataggtagcaatcagaaatttcctttgttatgtttggtgggagagaaacagactaaaaacttttctcgcaactggggtgtattgatagaagagaagggagatttgggggaggaaaaaaaccaggaaaaatgtttgatatttaaggataaaacagattttgctaaagctaaagaagtagaaatttttaaaaataatatttggcgtatcagaacctctaagtaccaaatccaatttttgaataggctttttaagaaaaccaaagaatgggatttaatgaatcttgtattgagcgagcctagcttagtattggaggaggaatggggtgtttcgtgggataaagataaacttttacctttactgaagaaagaaaaatcttgcgaagaaagattatattactcacttccccttaacttggtgcctgccacagattataaggagcaatctgcagaaatagagcaaaggaatacatatttgggtttggatgttggagaatttggtgttgcctatgcagtggtaagaatagtaagggacagaatagagcttctgtcctggggattccttaaggacccagctcttcgaaaaataagagagcgtgtacaggatatgaagaaaaagcaggtaatggcagtattttctagctcttccacagctgtcgcgcgagtacgagaaatggctatacactctttaagaaatcaaattcatagcattgctttggcgtataaagcaaagataatttatgagatatctataagcaattttgagacaggtggtaatagaatggctaaaatataccgatctataaaggtttcagatgtttatagggagagtggtgcggataccctagtttcagagatgatctggggcaaaaagaataagcaaatgggaaaccatatatcttcctatgcgacaagttacacttgttgcaattgtgcaagaaccccttttgaacttgttatagataatgacaaggaatatgaaaagggaggcgacgaatttatttttaatgttggcgatgaaaagaaggtaagggggtttttacaaaagagtctgttaggaaaaacaattaaagggaaggaagtgttgaagtctataaaagagtacgcaaggccgcctataagggaagtcttgcttgaaggagaagatgtagagcagttgttgaagaggagaggaaatagctatatttatagatgccctttttgtggatataaaactgatgcggatattcaagcggcgttgaatatagcttgtaggggatatatttcggataacgcaaaggatgctgtgaaggaaggagaaagaaaattagattacattttggaagttagaaaattgtgggagaagaatggagctgttttgagaagcgccaaatttttatagtt

SEQ ID NO: 11 - protein, 1226 aa, Artificial Sequence
  CasY.2
MQKVRKTLSEVHKNPYGTKVRNAKTGYSLQIERLSYTGKEGMRSFKIPLENKNKEVFDEFVKKIRNDYISQVGLLNLSDWYEHYQEKQEHYSLADFWLDSLRAGVIFAHKETEIKNLISKIRGDKSIVDKFNASIKKKHADLYALVDIKALYDFLTSDARRGLKTEEEFFNSKRNTLFPKFRKKDNKAVDLWVKKFIGLDNKDKLNFTKKFIGFDPNPQIKYDHTFFFHQDINFDLERITTPKELISTYKKFLGKNKDLYGSDETTEDQLKMVLGFHNNHGAFSKYFNASLEAFRGRDNSLVEQIINNSPYWNSHRKELEKRIIFLQVQSKKIKETELGKPHEYLASFGGKFESWVSNYLRQEEEVKRQLFGYEENKKGQKKFIVGNKQELDKIIRGTDEYEIKAISKETIGLTQKCLKLLEQLKDSVDDYTLSLYRQLIVELRIRLNVEFQETYPELIGKSEKDKEKDAKNKRADKRYPQIFKDIKLIPNFLGETKQMVYKKFIRSADILYEGINFIDQIDKQITQNLLPCFKNDKERIEFTEKQFETLRRKYYLMNSSRFHHVIEGIINNRKLIEMKKRENSELKTFSDSKFVLSKLFLKKGKKYENEVYYTFYINPKARDQRRIKIVLDINGNNSVGILQDLVQKLKPKWDDIIKKNDMGELIDAIEIEKVRLGILIALYCEHKFKIKKELLSLDLFASAYQYLELEDDPEELSGTNLGRFLQSLVCSEIKGAINKISRTEYIERYTVQPMNTEKNYPLLINKEGKATWHIAAKDDLSKKKGGGTVAMNQKIGKNFFGKQDYKTVFMLQDKRFDLLTSKYHLQFLSKTLDTGGGSWWKNKNIDLNLSSYSFIFEQKVKVEWDLTNLDHPIKIKPSENSDDRRLFVSIPFVIKPKQTKRKDLQTRVNYMGIDIGEYGLAWTIINIDLKNKKINKISKQGFIYEPLTHKVRDYVATIKDNQVRGTFGMPDTKLARLRENAITSLRNQVHDIAMRYDAKPVYEFEISNFETGSNKVKVIYDSVKRADIGRGQNNTEADNTEVNLVWGKTSKQFGSQIGAYATSYICSFCGYSPYYEFENSKSGDEEGARDNLYQMKKLSRPSLEDFLQGNPVYKTFRDFDKYKNDQRLQKTGDKDGEWKTHRGNTAIYACQKCRHISDADIQASYWIALKQVVRDFYKDKEMDGDLIQGDNKDKRKVNELNRLIGVHKDVPIINKNLITSLDINLL

SEQ ID NO: 12 - DNA, 2869 bp, Artificial Sequence
  CasY.2
atggtattaggttttcataataatcacggcgctttttctaagtatttcaacgcgagcttggaagcttttagggggagagacaactccttggttgaacaaataattaataattctccttactggaatagccatcggaaagaattggaaaagagaatcatttttttgcaagttcagtctaaaaaaataaaagagaccgaactgggaaagcctcacgagtatcttgcgagttttggcgggaagtttgaatcttgggtttcaaactatttacgtcaggaagaagaggtcaaacgtcaactttttggttatgaggagaataaaaaaggccagaaaaaatttatcgtgggcaacaaacaagagctagataaaatcatcagagggacagatgagtatgagattaaagcgatttctaaggaaaccattggacttactcagaaatgtttaaaattacttgaacaactaaaagatagtgtcgatgattatacacttagcctatatcggcaactcatagtcgaattgagaatcagactgaatgttgaattccaagaaacttatccggaattaatcggtaagagtgagaaagataaagaaaaagatgcgaaaaataaacgggcagacaagcgttacccgcaaatttttaaggatataaaattaatccccaattttctcggtgaaacgaaacaaatggtatataagaaatttattcgttccgctgacatcctttatgaaggaataaattttatcgaccagatcgataaacagattactcaaaatttgttgccttgttttaagaacgacaaggaacggattgaatttaccgaaaaacaatttgaaactttacggcgaaaatactatctgatgaatagttcccgttttcaccatgttattgaaggaataatcaataataggaaacttattgaaatgaaaaagagagaaaatagcgagttgaaaactttctccgatagtaagtttgttttatctaagctttttcttaaaaaaggcaaaaaatatgaaaatgaggtctattatactttttatataaatccgaaagctcgtgaccagcgacggataaaaattgttcttgatataaatgggaacaattcagtcggaattttacaagatcttgtccaaaagttgaaaccaaaatgggacgacatcataaagaaaaatgatatgggagaattaatcgatgcaatcgagattgagaaagtccggctcggcatcttgatagcgttatactgtgagcataaattcaaaattaaaaaagaactcttgtcattagatttgtttgccagtgcctatcaatatctagaattggaagatgaccctgaagaactttctgggacaaacctaggtcggtttttacaatccttggtctgctccgaaattaaaggtgcgattaataaaataagcaggacagaatatatagagcggtatactgtccagccgatgaatacggagaaaaactatcctttactcatcaataaggagggaaaagccacttggcatattgctgctaaggatgacttgtccaagaagaagggtgggggcactgtcgctatgaatcaaaaaatcggcaagaatttttttgggaaacaagattataaaactgtgtttatgcttcaggataagcggtttgatctactaacctcaaagtatcacttgcagtttttatctaaaactcttgatactggtggagggtcttggtggaaaaacaaaaatattgatttaaatttaagctcttattctttcattttcgaacaaaaagtaaaagtcgaatgggatttaaccaatcttgaccatcctataaagattaagcctagcgagaacagtgatgatagaaggcttttcgtatccattccttttgttattaaaccgaaacagacaaaaagaaaggatttgcaaactcgagtcaattatatggggattgatatcggagaatatggtttggcttggacaattattaatattgatttaaagaataaaaaaataaataagatttcaaaacaaggtttcatctatgagccgttgacacataaagtgcgcgattatgttgctaccattaaagataatcaggttagaggaacttttggcatgcctgatacgaaactagccagattgcgagaaaatgccattaccagcttgcgcaatcaagtgcatgatattgctatgcgctatgacgccaaaccggtatatgaatttgaaatttccaattttgaaacggggtctaataaagtgaaagtaatttatgattcggttaagcgagctgatatcggccgaggccagaataataccgaagcagacaatactgaggttaatcttgtctgggggaagacaagcaaacaatttggcagtcaaatcggcgcttatgcgacaagttacatctgttcattttgtggttattctccatattatgaatttgaaaattctaagtcgggagatgaagaaggggctagagataatctatatcagatgaagaaattgagtcgcccctctcttgaagatttcctccaaggaaatccggtttataagacatttagggattttgataagtataaaaacgatcaacggttgcaaaagacgggtgataaagatggtgaatggaaaacacacagagggaatactgcaatatacgcctgtcaaaagtgtagacatatctctgatgcggatatccaagcatcatattggattgctttgaagcaagttgtaagagatttttataaagacaaagagatggatggtgatttgattcaaggagataataaagacaagagaaaagtaaacgagcttaatagacttattggagtacataaagatgtgcctataataaataaaaatttaataacatcactcgacataaacttactataga

SEQ ID NO: 13 - protein, 1200 aa, Artificial Sequence
  CasY.3
MKAKKSFYNQKRKFGKRGYRLHDERIAYSGGIGSMRSIKYELKDSYGIAGLRNRIADATISDNKWLYGNINLNDYLEWRSSKTDKQIEDGDRESSLLGFWLEALRLGFVFSKQSHAPNDFNETALQDLFETLDDDLKHVLDRKKWCDFIKIGTPKTNDQGRLKKQIKNLLKGNKREEIEKTLNESDDELKEKINRIADVFAKNKSDKYTIFKLDKPNTEKYPRINDVQVAFFCHPDFEEITERDRTKTLDLIINRFNKRYEITENKKDDKTSNRMALYSLNQGYIPRVLNDLFLFVKDNEDDFSQFLSDLENFFSFSNEQIKIIKERLKKLKKYAEPIPGKPQLADKWDDYASDFGGKLESWYSNRIEKLKKIPESVSDLRNNLEKIRNVLKKQNNASKILELSQKIIEYIRDYGVSFEKPEIIKFSWINKTKDGQKKVFYVAKMADREFIEKLDLWMADLRSQLNEYNQDNKVSFKKKGKKIEELGVLDFALNKAKKNKSTKNENGWQQKLSESIQSAPLFFGEGNRVRNEEVYNLKDLLFSEIKNVENILMSSEAEDLKNIKIEYKEDGAKKGNYVLNVLARFYARFNEDGYGGWNKVKTVLENIAREAGTDFSKYGNNNNRNAGRFYLNGRERQVFTLIKFEKSITVEKILELVKLPSLLDEAYRDLVNENKNHKLRDVIQLSKTIMALVLSHSDKEKQIGGNYIHSKLSGYNALISKRDFISRYSVQTTNGTQCKLAIGKGKSKKGNEIDRYFYAFQFFKNDDSKINLKVIKNNSHKNIDFNDNENKINALQVYSSNYQIQFLDWFFEKHQGKKTSLEVGGSFTIAEKSLTIDWSGSNPRVGFKRSDTEEKRVFVSQPFTLIPDDEDKERRKERMIKTKNRFIGIDIGEYGLAWSLIEVDNGDKNNRGIRQLESGFITDNQQQVLKKNVKSWRQNQIRQTFTSPDTKIARLRESLIGSYKNQLESLMVAKKANLSFEYEVSGFEVGGKRVAKIYDSIKRGSVRKKDNNSQNDQSWGKKGINEWSFETTAAGTSQFCTHCKRWSSLAIVDIEEYELKDYNDNLFKVKINDGEVRLLGKKGWRSGEKIKGKELFGPVKDAMRPNVDGLGMKIVKRKYLKLDLRDWVSRYGNMAIFICPYVDCHHISHADKQAAFNIAVRGYLKSVNPDRAIKHGDKGLSRDFLCQEEGKLNFEQIGLL

SEQ ID NO: 14 - DNA, 3604 bp, Artificial Sequence
  CasY.3
atgaaagctaaaaaaagtttttataatcaaaagcggaagttcggtaaaagaggttatcgtcttcacgatgaacgtatcgcgtattcaggagggattggatcgatgcgatctattaaatatgaattgaaggattcgtatggaattgctgggcttcgtaatcgaatcgctgacgcaactatttctgataataagtggctgtacgggaatataaatctaaatgattatttagagtggcgatcttcaaagactgacaaacagattgaagacggagaccgagaatcatcactcctgggtttttggctggaagcgttacgactgggattcgtgttttcaaaacaatctcatgctccgaatgattttaacgagaccgctctacaagatttgtttgaaactcttgatgatgatttgaaacatgttcttgataggaaaaaatggtgtgactttatcaagataggaacacctaagacaaatgaccaaggtcgtttaaaaaaacaaatcaagaatttgttaaaaggaaacaagagagaggaaattgaaaaaactctcaatgaatcagacgatgaattgaaagagaaaataaacagaattgccgatgtttttgcaaaaaataagtctgataaatacacaattttcaaattagataaacccaatacggaaaaataccccagaatcaacgatgttcaggtggcgtttttttgtcatcccgattttgaggaaattacagaacgagatagaacaaagactctagatctgatcattaatcggtttaataagagatatgaaattaccgaaaataaaaaagatgacaaaacttcaaacaggatggccttgtattccttgaaccagggctatattcctcgcgtcctgaatgatttattcttgtttgtcaaagacaatgaggatgattttagtcagtttttatctgatttggagaatttcttctctttttccaacgaacaaattaaaataataaaggaaaggttaaaaaaacttaaaaaatatgctgaaccaattcccggaaagccgcaacttgctgataaatgggacgattatgcttctgattttggcggtaaattggaaagctggtactccaatcgaatagagaaattaaagaagattccggaaagcgtttccgatctgcggaataatttggaaaagatacgcaatgttttaaaaaaacaaaataatgcatctaaaatcctggagttatctcaaaagatcattgaatacatcagagattatggagtttcttttgaaaagccggagataattaagttcagctggataaataagacgaaggatggtcagaaaaaagttttctatgttgcgaaaatggcggatagagaattcatagaaaagcttgatttatggatggctgatttacgcagtcaattaaatgaatacaatcaagataataaagtttctttcaaaaagaaaggtaaaaaaatagaagagctcggtgtcttggattttgctcttaataaagcgaaaaaaaataaaagtacaaaaaatgaaaatggctggcaacaaaaattgtcagaatctattcaatctgccccgttattttttggcgaagggaatcgtgtacgaaatgaagaagtttataatttgaaggaccttctgttttcagaaatcaagaatgttgaaaatattttaatgagctcggaagcggaagacttaaaaaatataaaaattgaatataaagaagatggcgcgaaaaaagggaactatgtcttgaatgtcttggctagattttacgcgagattcaatgaggatggctatggtggttggaacaaagtaaaaaccgttttggaaaatattgcccgagaggcggggactgatttttcaaaatatggaaataataacaatagaaatgccggcagattttatctaaacggccgcgaacgacaagtttttactctaatcaagtttgaaaaaagtatcacggtggaaaaaatacttgaattggtaaaattacctagcctacttgatgaagcgtatagagatttagtcaacgaaaataaaaatcataaattacgcgacgtaattcaattgagcaagacaattatggctctggttttatctcattctgataaagaaaaacaaattggaggaaattatatccatagtaaattgagcggatacaatgcgcttatttcaaagcgagattttatctcgcggtatagcgtgcaaacgaccaacggaactcaatgtaaattagccataggaaaaggcaaaagcaaaaaaggtaatgaaattgacaggtatttctacgcttttcaattttttaagaatgacgacagcaaaattaatttaaaggtaatcaaaaataattcgcataaaaacatcgatttcaacgacaatgaaaataaaattaacgcattgcaagtgtattcatcaaactatcagattcaattcttagactggttttttgaaaaacatcaagggaagaaaacatcgctcgaggtcggcggatcttttaccatcgccgaaaagagtttgacaatagactggtcggggagtaatccgagagtcggttttaaaagaagcgacacggaagaaaagagggtttttgtctcgcaaccatttacattaataccagacgatgaagacaaagagcgtcgtaaagaaagaatgataaagacgaaaaaccgttttatcggtatcgatatcggtgaatatggtctggcttggagtctaatcgaagtggacaatggagataaaaataatagaggaattagacaacttgagagcggttttattacagacaatcagcagcaagtcttaaagaaaaacgtaaaatcctggaggcaaaaccaaattcgtcaaacgtttacttcaccagacacaaaaattgctcgtcttcgtgaaagtttgatcggaagttacaaaaatcaactggaaagtctgatggttgctaaaaaagcaaatcttagttttgaatacgaagtttccgggtttgaagttgggggaaagagggttgcaaaaatatacgatagtataaagcgtgggtcggtgcgtaaaaaggataataactcacaaaatgatcaaagttggggtaaaaagggaattaatgagtggtcattcgagacgacggctgccggaacatcgcaattttgtactcattgcaagcggtggagcagtttagcgatagtagatattgaagaatatgaattaaaagattacaacgataatttatttaaggtaaaaattaatgatggtgaagttcgtctccttggtaagaaaggttggagatccggcgaaaagatcaaagggaaagaattatttggtcccgtcaaagacgcaatgcgcccaaatgttgacggactagggatgaaaattgtaaaaagaaaatatctaaaacttgatctccgcgattgggtttcaagatatgggaatatggctattttcatctgtccttatgtcgattgccaccatatctctcatgcggataaacaagctgcttttaatattgccgtgcgagggtatttgaaaagcgttaatcctgacagagcaataaaacacggagataaaggtttgtctagggactttttgtgccaagaagagggtaagcttaattttgaacaaatagggttattatgaa

SEQ ID NO: 15 - protein, 1210 aa, Artificial Sequence
  CasY.4
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 16 - DNA, 3636 bp, Artificial Sequence
  CasY.4
atgagtaagcgacatcctagaattagcggcgtaaaagggtaccgtttgcatgcgcaacggctggaatataccggcaaaagtggggcaatgcgaacgattaaatatcctctttattcatctccgagcggtggaagaacggttccgcgcgagatagtttcagcaatcaatgatgattatgtagggctgtacggtttgagtaattttgacgatctgtataatgcggaaaagcgcaacgaagaaaaggtctactcggttttagatttttggtacgactgcgtccaatacggcgcggttttttcgtatacagcgccgggtcttttgaaaaatgttgccgaagttcgcgggggaagctacgaacttacaaaaacgcttaaagggagccatttatatgatgaattgcaaattgataaagtaattaaatttttgaataaaaaagaaatttcgcgagcaaacggatcgcttgataaactgaagaaagacatcattgattgcttcaaagcagaatatcgggaacgacataaagatcaatgcaataaactggctgatgatattaaaaatgcaaaaaaagacgcgggagcttctttaggggagcgtcaaaaaaaattatttcgcgatttttttggaatttcagagcagtctgaaaatgataaaccgtcttttactaatccgctaaacttaacctgctgtttattgccttttgacacagtgaataacaacagaaaccgcggcgaagttttgtttaacaagctcaaggaatatgctcaaaaattggataaaaacgaagggtcgcttgaaatgtgggaatatattggcatcgggaacagcggcactgccttttctaattttttaggagaagggtttttgggcagattgcgcgagaataaaattacagagctgaaaaaagccatgatggatattacagatgcatggcgtgggcaggaacaggaagaagagttagaaaaacgtctgcggatacttgccgcgcttaccataaaattgcgcgagccgaaatttgacaaccactggggagggtatcgcagtgatataaacggcaaattatctagctggcttcagaattacataaatcaaacagtcaaaatcaaagaggacttaaagggacacaaaaaggacctgaaaaaagcgaaagagatgataaataggtttggggaaagcgacacaaaggaagaggcggttgtttcatctttgcttgaaagcattgaaaaaattgttcctgatgatagcgctgatgacgagaaacccgatattccagctattgctatctatcgccgctttctttcggatggacgattaacattgaatcgctttgtccaaagagaagatgtgcaagaggcgctgataaaagaaagattggaagcggagaaaaagaaaaaaccgaaaaagcgaaaaaagaaaagtgacgctgaagatgaaaaagaaacaattgacttcaaggagttatttcctcatcttgccaaaccattaaaattggtgccaaacttttacggcgacagtaagcgtgagctgtacaagaaatataagaacgccgctatttatacagatgctctgtggaaagcagtggaaaaaatatacaaaagcgcgttctcgtcgtctctaaaaaattcattttttgatacagattttgataaagatttttttattaagcggcttcagaaaattttttcggtttatcgtcggtttaatacagacaaatggaaaccgattgtgaaaaactctttcgcgccctattgcgacatcgtctcacttgcggagaatgaagttttgtataaaccgaaacagtcgcgcagtagaaaatctgccgcgattgataaaaacagagtgcgtctcccttccactgaaaatatcgcaaaagctggcattgccctcgcgcgggagctttcagtcgcaggatttgactggaaagatttgttaaaaaaagaggagcatgaagaatacattgatctcatagaattgcacaaaaccgcgcttgcgcttcttcttgccgtaacagaaacacagcttgacataagcgcgttggattttgtagaaaatgggacggtcaaggattttatgaaaacgcgggacggcaatctggttttggaagggcgtttccttgaaatgttctcgcagtcaattgtgttttcagaattgcgcgggcttgcgggtttaatgagccgcaaggaatttatcactcgctccgcgattcaaactatgaacggcaaacaggcggagcttctctacattccgcatgaattccaatcggcaaaaattacaacgccaaaggaaatgagcagggcgtttcttgaccttgcgcccgcggaatttgctacatcgcttgagccagaatcgctttcggagaagtcattattgaaattgaagcagatgcggtactatccgcattattttggatatgagcttacgcgaacaggacaggggattgatggtggagtcgcggaaaatgcgttacgacttgagaagtcgccagtaaaaaaacgagagataaaatgcaaacagtataaaactttgggacgcggacaaaataaaatagtgttatatgtccgcagttcttattatcagacgcaatttttggaatggtttttgcatcggccgaaaaacgttcaaaccgatgttgcggttagcggttcgtttcttatcgacgaaaagaaagtaaaaactcgctggaattatgacgcgcttacagtcgcgcttgaaccagtttccggaagcgagcgggtctttgtctcacagccgtttactatttttccggaaaaaagcgcagaggaagaaggacagaggtatcttggcatagacatcggcgaatacggcattgcgtatactgcgcttgagataactggcgacagtgcaaagattcttgatcaaaattttatttcagacccccagcttaaaactctgcgcgaggaggtcaaaggattaaaacttgaccaaaggcgcgggacatttgccatgccaagcacgaaaatcgcccgcatccgcgaaagccttgtgcatagtttgcggaaccgcatacatcatcttgcgttaaagcacaaagcaaagattgtgtatgaattggaagtgtcgcgttttgaagagggaaagcaaaaaattaagaaagtctacgctacgttaaaaaaagcggatgtgtattcagaaattgacgcggataaaaatttacaaacgacagtatggggaaaattggccgttgcaagcgaaatcagcgcaagctatacaagccagttttgtggtgcgtgtaaaaaattgtggcgggcggaaatgcaggttgacgaaacaattacaacccaagaactaatcggcacagttagagtcataaaagggggcactcttattgacgcgataaaggattttatgcgcccgccgatttttgacgaaaatgacactccatttccaaaatatagagacttttgcgacaagcatcacatttccaaaaaaatgcgtggaaacagctgtttgttcatttgtccattctgccgcgcaaacgcggatgctgatattcaagcaagccaaacaattgcgcttttaaggtatgttaaggaagagaaaaaggtagaggactactttgaacgatttagaaagctaaaaaacattaaagtgctcggacagatgaagaaaatatgatag

SEQ ID NO: 17 - protein, 1192 aa, Artificial Sequence
  CasY.5
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDRGVAEKIEQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLQNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPAGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWSTYWEKRKPEDILGISTQVYWTGGIGESCPAVAVALLGHIRATSTQTEWEKEEVVFGRLKKFFPS

SEQ ID NO: 18 - DNA, 4560 bp, Artificial Sequence
  CasY.5
accaaccacctattgcgtctttttcgctcattttagcaaaagtggctgtctagacatacaggtggaaaggtgagagtaaagacatggcctgaatagcgtcctcgtcctcgtctagacatacaggtggaaaggtgagagtaaagaccggagcactcatcctctcactctattttgtctagacatacaggtggaaaggtgagagtaaagacaaaccgtgccacactaaaccgatgagtctagacatacaggtggaaaggtgagagtaaagactcaagtaactacctgttctttcacaagtctagacatacaggtggaaaggtgagagtaaagactcaagtaactacctgttctttcacaagtctagacctgcaggtggtaaggtgagagtaaagactcaagtaactacctgttctttcacaagtctagacctgcaggtggtaaggtgagagtaaagacttttatcctcctctctatgcttctgagtctagacatttaggtggaaaggtgagagtaaagacttgtggagatccatgaacttcggcagtctagacctgcaggtggaaaggtgagagtaaagacgtccttcacacgatcttcctctgttagtctaggcctgcaggtggaaaggtgagagtaaagacgcataagcgtaattgaagctctctccggtccagaccttgtcgcgcttgtgttgcgacaaaggcggagtccgcaataagttctttttacaatgttttttccataaaaccgatacaatcaagtatcggttttgctttttttatgaaaatatgttatgctatgtgctcaaataaaaatatcaataaaatagcgtttttttgataatttatcgctaaaattatacataatcacgcaacattgccattctcacacaggagaaaagtcatggcagaaagcaagcagatgcaatgccgcaagtgcggcgcaagcatgaagtatgaagtaattggattgggcaagaagtcatgcagatatatgtgcccagattgcggcaatcacaccagcgcgcgcaagattcagaacaagaaaaagcgcgacaaaaagtatggatccgcaagcaaagcgcagagccagaggatagctgtggctggcgcgctttatccagacaaaaaagtgcagaccataaagacctacaaatacccagcggatctgaatggcgaagttcatgacagaggcgtcgcagagaagattgagcaggcgattcaggaagatgagatcggcctgcttggcccgtccagcgaatacgcttgctggattgcttcacaaaaacaaagcgagccgtattcagttgtagatttttggtttgacgcggtgtgcgcaggcggagtattcgcgtattctggcgcgcgcctgctttccacagtcctccagttgagtggcgaggaaagcgttttgcgcgctgctttagcatctagcccgtttgtagatgacattaatttggcgcaagcggaaaagttcctagccgttagccggcgcacaggccaagataagctaggcaagcgcattggagaatgtttcgcggaaggccggcttgaagcgcttggcatcaaagatcgcatgcgcgaattcgtgcaagcgattgatgtggcccaaaccgcgggccagcggttcgcggccaagctaaagatattcggcatcagtcagatgcctgaagccaagcaatggaacaatgattccgggctcactgtatgtattttgccggattattatgtcccggaagaaaaccgcgcggaccagctggttgttttgcttcggcgcttacgcgagatcgcgtattgcatgggaattgaggatgaagcaggatttgagcatctaggcattgaccctggcgctctttccaatttttccaatggcaatccaaagcgaggatttctcggccgcctgctcaataatgacattatagcgctggcaaacaacatgtcagccatgacgccgtattgggaaggcagaaaaggcgagttgattgagcgccttgcatggcttaaacatcgcgctgaaggattgtatttgaaagagccacatttcggcaactcctgggcagaccaccgcagcaggattttcagtcgcattgcgggctggctttccggatgcgcgggcaagctcaagattgccaaggatcagatttcaggcgtgcgtacggatttgtttctgctcaagcgccttctggatgcggtaccgcaaagcgcgccgtcgccggactttattgcttccatcagcgcgctggatcggtttttggaagcggcagaaagcagccaggatccggcagaacaggtacgcgctttgtacgcgtttcatctgaacgcgcctgcggtccgatccatcgccaacaaggcggtacagaggtctgattcccaggagtggcttatcaaggaactggatgctgtagatcaccttgaattcaacaaagcatttccgtttttttcggatacaggaaagaaaaagaagaaaggagcgaatagcaacggagcgccttctgaagaagaatacacggaaacagaatccattcaacaaccagaagatgcagagcaggaagtgaatggtcaagaaggaaatggcgcttcaaagaaccagaaaaagtttcagcgcattcctcgatttttcggggaagggtcaaggagtgagtatcgaattttaacagaagcgccgcaatattttgacatgttctgcaataatatgcgcgcgatctttatgcagctagagagtcagccgcgcaaggcgcctcgtgatttcaaatgctttctgcagaatcgtttgcagaagctttacaagcaaacctttctcaatgctcgcagtaataaatgccgcgcgcttctggaatccgtccttatttcatggggagaattttatacttatggcgcgaatgaaaagaagtttcgtctgcgccatgaagcgagcgagcgcagctcggatccggactatgtggttcagcaggcattggaaatcgcgcgccggcttttcttgttcggatttgagtggcgcgattgctctgctggagagcgcgtggatttggttgaaatccacaaaaaagcaatctcatttttgcttgcaatcactcaggccgaggtttcagttggttcctataactggcttgggaatagcaccgtgagccggtatctttcggttgctggcacagacacattgtacggcactcaactggaggagtttttgaacgccacagtgctttcacagatgcgtgggctggcgattcggctttcatctcaggagttaaaagacggatttgatgttcagttggagagttcgtgccaggacaatctccagcatctgctggtgtatcgcgcttcgcgcgacttggctgcgtgcaaacgcgctacatgcccggctgaattggatccgaaaattcttgttctgccggctggtgcgtttatcgcgagcgtaatgaaaatgattgagcgtggcgatgaaccattagcaggcgcgtatttgcgtcatcggccgcattcattcggctggcagatacgggttcgtggagtggcggaagtaggcatggatcagggcacagcgctagcattccagaagccgactgaatcagagccgtttaaaataaagccgttttccgctcaatacggcccagtactttggcttaattcttcatcctatagccagagccagtatctggatggatttttaagccagccaaagaattggtctatgcgggtgctacctcaagccggatcagtgcgcgtggaacagcgcgttgctctgatatggaatttgcaggcaggcaagatgcggctggagcgctctggagcgcgcgcgtttttcatgccagtgccattcagcttcaggccgtctggttcaggagatgaagcagtattggcgccgaatcggtacttgggactttttccgcattccggaggaatagaatacgcggtggtggatgtattagattccgcgggtttcaaaattcttgagcgcggtacgattgcggtaaatggcttttcccagaagcgcggcgaacgccaagaggaggcacacagagaaaaacagagacgcggaatttctgatataggccgcaagaagccggtgcaagctgaagttgacgcagccaatgaattgcaccgcaaatacaccgatgttgccactcgtttagggtgcagaattgtggttcagtgggcgccccagccaaagccgggcacagcgccgaccgcgcaaacagtatacgcgcgcgcagtgcggaccgaagcgccgcgatctggaaatcaagaggatcatgctcgtatgaaatcctcttggggatatacctggagcacctattgggagaagcgcaaaccagaggatattttgggcatctcaacccaagtatactggaccggcggtataggcgagtcatgtcccgcagtcgcggttgcgcttttggggcacattagggcaacatccactcaaactgaatgggaaaaagaggaggttgtattcggtcgactgaagaagttctttccaagctagacgatctttttaaaaactgggctgctggctatcgtatggtcagtagctcttatttttttacttgatatatggtattat

SEQ ID NO: 19 - protein, 1287 aa, Artificial Sequence
  CasY.6
MKRILNSLKVAALRLLFRGKGSELVKTVKYPLVSPVQGAVEELAEAIRHDNLHLFGQKEIVDLMEKDEGTQVYSVVDFWLDTLRLGMFFSPSANALKITLGKFNSDQVSPFRKVLEQSPFFLAGRLKVEPAERILSVEIRKIGKRENRVENYAADVETCFIGQLSSDEKQSIQKLANDIWDSKDHEEQRMLKADFFAIPLIKDPKAVTEEDPENETAGKQKPLELCVCLVPELYTRGFGSIADFLVQRLTLLRDKMSTDTAEDCLEYVGIEEEKGNGMNSLLGTFLKNLQGDGFEQIFQFMLGSYVGWQGKEDVLRERLDLLAEKVKRLPKPKFAGEWSGHRMFLHGQLKSWSSNFFRLFNETRELLESIKSDIQHATMLISYVEEKGGYHPQLLSQYRKLMEQLPALRTKVLDPEIEMTHMSEAVRSYIMIHKSVAGFLPDLLESLDRDKDREFLLSIFPRIPKIDKKTKEIVAWELPGEPEEGYLFTANNLFRNFLENPKHVPRFMAERIPEDWTRLRSAPVWFDGMVKQWQKVVNQLVESPGALYQFNESFLRQRLQAMLTVYKRDLQTEKFLKLLADVCRPLVDFFGLGGNDIIFKSCQDPRKQWQTVIPLSVPADVYTACEGLAIRLRETLGFEWKNLKGHEREDFLRLHQLLGNLLFWIRDAKLVVKLEDWMNNPCVQEYVEARKAIDLPLEIFGFEVPIFLNGYLFSELRQLELLLRRKSVMTSYSVKTTGSPNRLFQLVYLPLNPSDPEKKNSNNFQERLDTPTGLSRRFLDLTLDAFAGKLLTDPVTQELKTMAGFYDHLFGFKLPCKLAAMSNHPGSSSKMVVLAKPKKGVASNIGFEPIPDPAHPVFRVRSSWPELKYLEGLLYLPEDTPLTIELAETSVSCQSVSSVAFDLKNLTTILGRVGEFRVTADQPFKLTPIIPEKEESFIGKTYLGLDAGERSGVGFAIVTVDGDGYEVQRLGVHEDTQLMALQQVASKSLKEPVFQPLRKGTFRQQERIRKSLRGCYWNFYHALMIKYRAKVVHEESVGSSGLVGQWLRAFQKDLKKADVLPKKGGKNGVDKKKRESSAQDTLWGGAFSKKEEQQIAFEVQAAGSSQFCLKCGWWFQLGMREVNRVQESGVVLDWNRSIVTFLIESSGEKVYGFSPQQLEKGFRPDIETFKKMVRDFMRPPMFDRKGRPAAAYERFVLGRRHRRYRFDKVFEERFGRSALFICPRVGCGNFDHSSEQSAVVLALIGYIADKEGMSGKKLVYVRLAELMAEWKLKKLERSRVEEQSSAQ

SEQ ID NO: 20 - DNA, 3864 bp, Artificial Sequence
  CasY.6
atgaagagaattctgaacagtctgaaagttgctgccttgagacttctgtttcgaggcaaaggttctgaattagtgaagacagtcaaatatccattggtttccccggttcaaggcgcggttgaagaacttgctgaagcaattcggcacgacaacctgcacctttttgggcagaaggaaatagtggatcttatggagaaagacgaaggaacccaggtgtattcggttgtggatttttggttggataccctgcgtttagggatgtttttctcaccatcagcgaatgcgttgaaaatcacgctgggaaaattcaattctgatcaggtttcaccttttcgtaaggttttggagcagtcacctttttttcttgcgggtcgcttgaaggttgaacctgcggaaaggatactttctgttgaaatcagaaagattggtaaaagagaaaacagagttgagaactatgccgccgatgtggagacatgcttcattggtcagctttcttcagatgagaaacagagtatccagaagctggcaaatgatatctgggatagcaaggatcatgaggaacagagaatgttgaaggcggatttttttgctatacctcttataaaagaccccaaagctgtcacagaagaagatcctgaaaatgaaacggcgggaaaacagaaaccgcttgaattatgtgtttgtcttgttcctgagttgtatacccgaggtttcggctccattgctgattttctggttcagcgacttaccttgctgcgtgacaaaatgagtaccgacacggcggaagattgcctcgagtatgttggcattgaggaagaaaaaggcaatggaatgaattccttgctcggcacttttttgaagaacctgcagggtgatggttttgaacagatttttcagtttatgcttgggtcttatgttggctggcaggggaaggaagatgtactgcgcgaacgattggatttgctggccgaaaaagtcaaaagattaccaaagccaaaatttgccggagaatggagtggtcatcgtatgtttctccatggtcagctgaaaagctggtcgtcgaatttcttccgtctttttaatgagacgcgggaacttctggaaagtatcaagagtgatattcaacatgccaccatgctcattagctatgtggaagagaaaggaggctatcatccacagctgttgagtcagtatcggaagttaatggaacaattaccggcgttgcggactaaggttttggatcctgagattgagatgacgcatatgtccgaggctgttcgaagttacattatgatacacaagtctgtagcgggatttctgccggatttactcgagtctttggatcgagataaggatagggaatttttgctttccatctttcctcgtattccaaagatagataagaagacgaaagagatcgttgcatgggagctaccgggcgagccagaggaaggctatttgttcacagcaaacaaccttttccggaattttcttgagaatccgaaacatgtgccacgatttatggcagagaggattcccgaggattggacgcgtttgcgctcggcccctgtgtggtttgatgggatggtgaagcaatggcagaaggtggtgaatcagttggttgaatctccaggcgccctttatcagttcaatgaaagttttttgcgtcaaagactgcaagcaatgcttacggtctataagcgggatctccagactgagaagtttctgaagctgctggctgatgtctgtcgtccactcgttgattttttcggacttggaggaaatgatattatcttcaagtcatgtcaggatccaagaaagcaatggcagactgttattccactcagtgtcccagcggatgtttatacagcatgtgaaggcttggctattcgtctccgcgaaactcttggattcgaatggaaaaatctgaaaggacacgagcgggaagattttttacggctgcatcagttgctgggaaatctgctgttctggatcagggatgcgaaacttgtcgtgaagctggaagactggatgaacaatccttgtgttcaggagtatgtggaagcacgaaaagccattgatcttcccttggagattttcggatttgaggtgccgatttttctcaatggctatctcttttcggaactgcgccagctggaattgttgctgaggcgtaagtcggtgatgacgtcttacagcgtcaaaacgacaggctcgccaaataggctcttccagttggtttacctacctctaaacccttcagatccggaaaagaaaaattccaacaactttcaggagcgcctcgatacacctaccggtttgtcgcgtcgttttctggatcttacgctggatgcatttgctggcaaactcttgacggatccggtaactcaggaactgaagacgatggccggtttttacgatcatctctttggcttcaagttgccgtgtaaactggcggcgatgagtaaccatccaggatcctcttccaaaatggtggttctggcaaaaccaaagaagggtgttgctagtaacatcggctttgaacctattcccgatcctgctcatcctgtgttccgggtgagaagttcctggccggagttgaagtacctggaggggttgttgtatcttcccgaagatacaccactgaccattgaactggcggaaacgtcggtcagttgtcagtctgtgagttcagtcgctttcgatttgaagaatctgacgactatcttgggtcgtgttggtgaattcagggtgacggcagatcaacctttcaagctgacgcccattattcctgagaaagaggaatccttcatcgggaagacctacctcggtcttgatgctggagagcgatctggcgttggtttcgcgattgtgacggttgacggcgatgggtatgaggtgcagaggttgggtgtgcatgaagatactcagcttatggcgcttcagcaagtcgccagcaagtctcttaaggagccggttttccagccactccgtaagggcacatttcgtcagcaggagcgcattcgcaaaagcctccgcggttgctactggaatttctatcatgcattgatgatcaagtaccgagctaaagttgtgcatgaggaatcggtgggttcatccggtctggtggggcagtggctgcgtgcatttcagaaggatctcaaaaaggctgatgttctgcccaagaagggtggaaaaaatggtgtagacaaaaaaaagagagaaagcagcgctcaggataccttatggggaggagctttctcgaagaaggaagagcagcagatagcctttgaggttcaggcagctggatcaagccagttttgtctgaagtgtggttggtggtttcagttggggatgcgggaagtaaatcgtgtgcaggagagtggcgtggtgctggactggaaccggtccattgtaaccttcctcatcgaatcctcaggagaaaaggtatatggtttcagtcctcagcaactggaaaaaggctttcgtcctgacatcgaaacgttcaaaaaaatggtaagggattttatgagaccccccatgtttgatcgcaaaggtcggccggccgcggcgtatgaaagattcgtactgggacgtcgtcaccgtcgttatcgctttgataaagtttttgaagagagatttggtcgcagtgctcttttcatctgcccgcgggtcgggtgtgggaatttcgatcactccagtgagcagtcagccgttgtccttgcccttattggttacattgctgataaggaagggatgagtggtaagaagcttgtttatgtgaggctggctgaacttatggctgagtggaagctgaagaaactggagagatcaagggtggaagaacagagctcggcacaataa